(19) United States
(12) Patent Application Publication
(10) Pub. No.: US 2025/0333703 A1
(43) Pub. Date: Oct. 30, 2025

(54) Materials, Methods and Systems for Cellular Redifferentiation and Expansion

(71) Applicant: Janssen Biotech, Inc., Horsham, PA (US)

(72) Inventors: Sandra Hayes, Doylestown, PA (US); Naman PATEL, North Brunswick, NJ (US); Marisa DEVENNY, Springfield, PA (US); Dharmeshkumar PATEL, Lansdale, PA (US)

(21) Appl. No.: 19/237,596

(22) Filed: Jun. 13, 2025

Related U.S. Application Data
(63) Continuation of application No. PCT/IB2023/062736, filed on Dec. 15, 2023.
(60) Provisional application No. 63/387,544, filed on Dec. 15, 2022.
(60) Provisional application No. 63/387,542, filed on Dec. 15, 2022.
(60) Provisional application No. 63/387,541, filed on Dec. 15, 2022.
(60) Provisional application No. 63/470,616, filed on Jun. 2, 2023.

(51) Int. Cl. C12N 5/0789; C12M 1/00; C12M 1/34; C12M 1/36; C12N 5/00

(52) U.S. Cl. CPC C12N 5/0647; C12M 41/14; C12M 41/36; C12M 41/44; C12M 41/48; C12N 5/0031; C12N 2500/32; C12N 2501/115; C12N 2501/125; C12N 2501/14; C12N 2501/15; C12N 2501/155; C12N 2501/16; C12N 2501/165; C12N 2501/2303; C12N 2501/2306; C12N 2501/26; C12N 2501/727; C12N 2501/999; C12N 2506/45; C12N 2509/00; C12N 2527/00

(57) ABSTRACT

Materials, methods, and systems for the cellular redifferentiation and expansion of induced pluripotent stem cell (iPSC)-derived hematopoietic stem cells (iHSCs) without the use of serum or additional cells is provided.

### CROSS-REFERENCE TO RELATED APPLICATIONS

This application claims priority to, and the benefit of, U.S. Provisional Application Nos. 63/387,541; 63/387,542; and 63/387,544, all filed Dec. 15, 2022, and 63/470,616 filed Jun. 2, 2023, the contents of each of which are incorporated herein in their entireties as if fully set forth herein.

### FIELD

The present invention teaches cell production, including for example materials, methods, and systems for the cellular growth, redifferentiation and expansion of induced pluripotent stem cell (iPSC)-derived hematopoietic stem cells (e.g., iHSCs), which are useful in, inter alia, producing T cells, such as gamma delta (γδ) T cells, derived from such iPSCs, including sustainable cellular methodologies and strategies. The γδ T cells produced by these methods can be gene edited. The materials, methods, and systems of the present invention advantageously reduce or eliminate the use of costly materials, energy, carbon feedstock(s), and biomass, etc., such as animal serum, additional cells (e.g., feeder cells or stromal cells), human supervision, and the like, in iHSC manufacture and use.

### BACKGROUND

Cell therapy, such as immune T cell immunotherapy, including autologous chimeric antigen receptor (CAR) T cell therapies, has been reported to be an efficacious therapy for the treatment of some diseases, such as some cancers, including some hematologic malignancies. However, applications of cell therapy to treat multiple cancer types has met numerous and varied challenges (see, e.g., Advances and challenges of CAR T therapy and suitability of animal models (Review), Authors: Xavier E. Ramos-Cardona Weichuan Luo Sulma I. Mohammed; Published online on: Jul. 12, 2022).

### SUMMARY

Advances in T cell immunotherapy have been reported, such as in autologous CAR T cell therapy; however, applications of cell therapy to treat multiple cancer types has numerous unmet challenges; and the present invention, against this backdrop, meets many of the challenges that impede access to obtaining therapeutic cell products, such as for example prohibitive, high manufacturing costs, undue complexity, lack of consistent and scalable manufacturing processes, automation, avoidance of scarce resources, etc. For example, the inventors of the present invention use iPSCs, which can undergo, inter alia, self-renewal, gene editing, multilineage differentiation, etc., to advance off-the-shelf, allogeneic, T cell therapy platforms with more commercially viable, automated, energy efficient, and resource sparing manufacturing processes. For example, an iPSC-derived cell therapy platform is based on production of γδ T cells, in accordance with the present invention, which cell therapy platform possesses intrinsic antitumor activity and whose cellular tumor infiltration is associated with more favorable outcomes. There is therefore a need in the art for methods of efficiently redifferentiating iPSCs into iPSC-derived HSCs (iHSCs) as a source for manufacturing robust allogeneic γδ T cells in sufficient numbers met by the present invention. Such iHSCs are of sufficient purity and fold expansion, and with high T cell redifferentiation potential for use for T cell-based products, as well as γδ T cells that have superior increased purity and fold expansion properties and are more readily made and used. The present invention is directed to such improved methods and materials and systems.

Furthermore, the inventors address a need in the art for more sustainable methods of generating iHSCs from iPSCs without the use of, for example, biologic reagents, serum, and/or biomass, such as additional cells (e.g., feeder or stromal cells) and cell extracts that result in iHSCs with high purity, viability, fold expansion, and potential to redifferentiate into immune effector cells, including lymphocytes such as γδ T cells, specifically iPSC-derived γδ (iγδ) T cells. The present application addresses these and other needs.

The invention taught herein has multiple aspects. In an aspect, the present invention provides a method of de novo generation of hematopoietic stem cells (iHSCs) derived from human γδ T cell derived induced pluripotent stem cells (iPSCs) under serum-free and feeder-free culture conditions, said method comprising: (i) seeding human γδ T cell derived iPSCs in a tissue culture vessel wherein the iPSCs are maintained for up to about 6 days; (ii) initiating redifferentiation of the iPSCs into iHSCs by culturing the iPSCs in the tissue culture vessel for about 2 days to obtain iPSC-derived cell intermediates; (iii) culturing the iPSC-derived cell intermediates for about 8 days; (iv) collecting non-adherent iPSC-derived cell intermediates and adding them back to the culture; and (v) harvesting redifferentiated iHSCs at about day 10 of culture into two different cellular fractions, a non-adherent cellular fraction and an adherent cellular fraction.

In some embodiments, the methods further comprise initiating redifferentiation of the iPSCs in a composition comprising basic fibroblast growth factor (bFGF), a glycogen synthase kinase inhibitor, a bone morphogenetic protein (BMP4), and vascular endothelial growth factor (VEGF).

In some embodiments, the methods further comprise culturing the iPSC-derived cell intermediates for about 2 days in a composition comprising effective amounts of L-glutamine, an inhibitor of the Activin/BMP/TGFβ pathway, VEGF, bFGF, and, optionally, a stem cell factor (SCF) and/or antibiotic(s).

In some embodiments, the methods further comprise culturing the iPSC-derived cell intermediates for about 2 days in a composition comprising L-glutamine, SCF, VEGF, FMS-like tyrosine kinase 3 ligand (Flt3L), erythropoietin (EPO), interleukin-3 (IL-3), interleukin-6 (IL-6), and optionally antibiotic(s).

In some embodiments, the methods further comprise culturing the iPSC-derived cell intermediates for about 2 days in a composition comprising L-glutamine, SCF, IL-6, EPO, and optionally antibiotic(s).

In some embodiments, the methods further comprise culturing redifferentiated iHSCs for about 2 days in a composition comprising L-glutamine and optionally antibiotic(s), wherein the composition does not comprise cytokines or growth factors.

In one aspect, the invention provides a method of de novo generation of hematopoietic stem cells (iHSCs) derived from human γδ T cell derived induced pluripotent stem cells (iPSCs) under serum-free, feeder-free culture conditions, said method comprising:
i) obtaining one or more human γδ T cell derived induced pluripotent stem cells (iPSCs), and
   wherein the method further comprises one or more of the following steps:
a) seeding the iPSCs at about 1.7×10³ cells/well to about 2.5×10³ cells/well, preferably about 2×10³ cells/well, of a six-well tissue culture vessel or an equivalent thereof;
b) maintaining the iPSCs in culture conditions for about 5.5 to about 6.5 days, preferably up to 6 days;
c) initiating redifferentiation of the iPSCs into iHSCs by culturing the iPSCs for about 1.8 days to about 2.2 days, preferably about 2 days, in a first cell culture medium to obtain iPSC-derived cell intermediates, wherein said first cell culture medium comprises StemFit® Basic 04 medium supplemented with basic fibroblast growth factor (bFGF) to a final concentration of about 90 ng/mL to about 110 ng/mL bFGF, preferably about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the iPSC-derived cell intermediates for an additional about 1.8 days to about 2.2 days, preferably about 2 days, in a second cell culture medium, wherein said second cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein the SB431542 has a final concentration of about 1.5 μM to about 2.5 μM;
e) culturing the iPSC-derived cell intermediates for an additional about 1.8 days to about 2.2 days, preferably about 2 days, in a third cell culture medium, wherein said third cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the iPSC-derived cell intermediates for an additional about 1.8 days to about 4.2 days, preferably about 2 days to about 4 days, in a fourth cell culture medium, wherein said fourth cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SCF, IL-6, EPO, and optionally penicillin/streptomycin;
g) after about 7.8 days to about 8.2 days, preferably 8 days, of redifferentiation, collecting non-adherent iPSC-derived cell intermediates in the fourth cell culture medium and adding them back to the culture in the fourth cell culture medium;
h) harvesting redifferentiated iHSCs at Day 10 of culture in two different cellular fractions, a non-adherent cellular fraction and an adherent cellular fraction, wherein the redifferentiated iHSCs are harvested in a fifth cell culture medium, wherein the fifth cell culture medium comprises Advanced DMEM/F12 medium and StemFit® For Differentiation medium in about a 3.9 to 0.9 ratio to about a 4.1 to 1.1 ratio, preferably about a 4:1 ratio, and further comprises L-glutamine and optionally penicillin/streptomycin, wherein the fifth cell culture medium does not comprise cytokines or growth factors;
i) harvesting non-adherent iHSCs in the medium by centrifugation, wherein said harvesting optionally does not include further culturing;
j) harvesting adherent iHSCs by dispersing cells, optionally resuspending the cells obtained from dispersion, followed by passing through a first cell strainer and a second cell strainer, wherein the first cell strainer is 70 μm mesh size and the second cell strainer is 40 μm mesh size;
k) determining the purity of the iHSCs, said purity defined as CD34+ and lineage marker-negative;
l) not introducing serum in any of steps a-k;
m) not introducing any additional cells, including feeder cells or stromal cells, in any of steps a-1; or
n) obtaining de novo generated iHSCs.

In an aspect, the present invention comprises a method of de novo generation of hematopoietic stem cells (iHSCs) derived from human γδT cell derived induced pluripotent stem cell (iPSC) under serum-free and feeder-free culture conditions, said method comprising: a) seeding the iPSCs at about 0.4×10⁶ cells/well to about 1.2×10⁶ cells/well of a six-well ultra low adhesion tissue culture vessel or an equivalent thereof; b) culturing the iPSCs for up to about 48 hours with agitation to generate embryoid bodies (EBs); c) transferring the EBs to a tissue culture treated culture vessel and initiating redifferentiation of the EBs in the tissue culture treated culture vessel for about 10 days, wherein the tissue culture treated culture vessel comprises EBs and cells in suspension; d) obtaining the EBs and cells in suspension; and e) optionally, treating the EBs and cells in suspension to obtain a single cell suspension.

In some embodiments, the present invention further comprises obtaining CD34+ and lineage marker-negative iHSCs.

In some embodiments, the present invention further comprises obtaining iHSCs that are CD34+, and CD3−, and/or CD19−, and/or CD14−, and/or CD11b−, and/or CD11c−, and/or CD56−, and/or CD20−, and/or CD16−, and/or CD2−, and/or CD235a− cells.

In another aspect, the invention provides a method of de novo generation of hematopoietic stem cells (iHSCs) derived from human γδ T cell derived induced pluripotent stem cells (iPSCs) under serum-free, feeder-free culture conditions, said method comprising:
i) obtaining one or more human γδ T cell derived induced pluripotent stem cells (iPSCs), and
   wherein the method further comprises one or more of the following steps:
a) seeding the iPSCs at about 0.55×10⁶ cells/well to about 0.65×10⁶ cells/well, preferably about 0.6×10⁶ cells/well, of a six-well ultra low adhesion tissue culture vessel or an equivalent thereof;
b) maintaining iPSCs in culture conditions for about 44 hours to about 52 hours, preferably up to 48 hours, with agitation wherein embryoid bodies (EBs) are generated;
c) transferring the EBs from the ultra low adhesion tissue culture vessel to a tissue culture treated tissue culture vessel and redifferentiating the EBs by culturing the EBs for about 1.8 days to about 2.2 days, preferably about 2 days, in a first cell culture medium, wherein said first cell culture medium comprises StemFit® Basic 04 medium supplemented with basic fibroblast growth factor (bFGF) to a final concentration of about 90 ng/mL to about 110 ng/mL bFGF, preferably about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing EBs and cells in suspension for an additional about 1.8 days to about 2.2 days, preferably about 2 days, in a second cell culture medium, wherein said second cell culture medium comprises Advanced DMEM/F12 medium and StemFit® For Differentiation medium present in about a 3.9-0.9 ratio to about a 4.1-1.1 ratio, preferably about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein the SB431542 has a final concentration of about 1.5 μM to about 2.5 μM;
e) culturing the EBs and cells in suspension for an additional about 1.8 days to about 2.2 days, preferably about 2 days, in a third cell culture medium, wherein said third cell culture medium comprises Advanced DMEM/F12 medium and StemFit® For Differentiation medium present in about a 3.9-0.9 ratio to a 4.1-1.1 ratio, preferably about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the EBs and cells in suspension for an additional about 1.8 days to about 4.2 days, preferably about 2 days to about 4 days, in a fourth cell culture medium, wherein said fourth cell culture medium comprises Advanced DMEM/F12 medium and StemFit® For Differentiation medium present in about a 3.9-0.9 ratio to a 4.1-1.1 ratio, preferably about a 4:1 ratio, and further comprises L-glutamine, SCF, IL-6, EPO, and optionally penicillin/streptomycin;
g) after about 10 days of redifferentiation, harvesting the EBs and cells in suspension by centrifugation, thereby forming pelleted EBs and cells in suspension;
h) resuspending the pelleted EBs and cells in suspension in a volume of 18 mg/mL to about 20 mg/mL collagenase type II and incubating the EBs and cells in suspension in the collagenase type II;
i) adding TrypLE to the EBs and cells in suspension and incubating the EBs and cells in suspension to obtain a single cell suspension,
j) mixing the single cell suspension;
k) adding a fifth cell culture medium to the cell suspension, wherein said fifth cell culture medium comprises Advanced DMEM/F12 medium and StemFit® For Differentiation medium in a ratio of about a 3.9-0.9 ratio to about a 4.1-1.1 ratio, preferably about a 4:1 ratio, and further comprises L-glutamine and optionally penicillin/streptomycin, wherein the fifth cell culture medium does not comprise cytokines or growth factors,
l) centrifuging the cell suspension to form a cell pellet and resuspending the cell pellet in the fifth cell culture medium to form a second cell suspension,
m) passing the second cell suspension through a first cell strainer and a second cell strainer to obtain iHSCs, wherein the first cell strainer is 70 μm mesh size and the second cell strainer is 40 μm mesh size;
n) determining the purity of the iHSCs, said purity defined as CD34+ and lineage marker-negative;
o) not introducing serum in any of steps a-n;
p) not introducing any additional cells, including feeder cells or stromal cells, in any of steps a-o; or
q) obtaining de novo generated iHSCs.

In any of the embodiments herein, the cells are cultured under normoxic conditions.

In any of the embodiments herein, the method further comprises further purifying and/or isolating the iHSCs.

In any of the embodiments herein, the obtained iHSCs are isolated differentiated cells or are capable of being further purified and/or isolated.

In any of the embodiments herein, the step of determining the purity of the iHSCs comprises assaying for one or more, optionally all, of the lineage markers CD3, CD19, CD14, CD11b, CD11c, CD56, CD20, CD16, CD2, and CD235a via flow cytometry or an equivalent thereof.

In any of the embodiments herein, the lineage markers comprise one or more of CD3, CD19, CD14, CD11b, CD11c, CD56, CD20, CD16, CD2, and CD235a.

In any of the embodiments herein, the step of resuspending the cells comprises gently dispersing the cells using a pipet or equivalent thereof.

In another aspect, the invention provides one or more cells obtained from any of the methods described herein.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method comprising: seeding human γδ T cell derived iPSCs in a tissue culture vessel wherein the iPSCs are maintained for up to about 6 days; initiating redifferentiation of the iPSCs into iHSCs by culturing the iPSCs in the tissue culture vessel for about 2 days to obtain iPSC-derived cell intermediates; culturing the iPSC-derived cell intermediates for about 8 days; collecting non-adherent iPSC-derived cell intermediates and adding them back to the culture; and harvesting redifferentiated iHSCs at about day 10 of culture into two different cellular fractions, a non-adherent cellular fraction and an adherent cellular fraction.

In some embodiments, the method of making the cells further comprises initiating redifferentiation of the iPSCs in a composition comprising basic fibroblast growth factor (bFGF), a glycogen synthase kinase inhibitor, a bone morphogenetic protein (BMP4), and vascular endothelial growth factor (VEGF).

In some embodiments, the method of making the cells further comprises culturing the iPSC-derived cell intermediates for about 2 days in a composition comprising effective amounts of L-glutamine, an inhibitor of the Activin/BMP/TGFβ pathway), VEGF, bFGF, and, optionally, a stem cell factor (SCF) and/or antibiotic(s).

In some embodiments, the methods further comprise culturing the iPSC-derived cell intermediates for about 2 days in a composition comprising L-glutamine, SCF, VEGF, FMS-like tyrosine kinase 3 ligand (Flt3L), erythropoietin (EPO), interleukin-3 (IL-3), interleukin-6 (IL-6), and optionally antibiotic(s).

In some embodiments, the methods further comprise culturing the iPSC-derived cell intermediates for about 2 days in a composition comprising L-glutamine, SCF, IL-6, EPO, and optionally antibiotic(s).

In some embodiments, the methods further comprise culturing redifferentiated iHSCs for about 2 days in a composition comprising L-glutamine and optionally antibiotic(s), wherein the composition does not comprise cytokines or growth factors.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, wherein said method comprises:
i) obtaining at least one human γδ T cell derived iPSC, and
wherein the method further comprises one or more of the following steps:
a) seeding the iPSCs at about 1.7×10³ cells/well to about 2.5×10³ cells per well, preferably about 2×10³ cells/well of a six-well tissue culture vessel or an equivalent thereof;
b) maintaining the iPSCs under culture conditions for up to 6 days;
c) initiating redifferentiation of the iPSCs to iHSCs by culturing the iPSCs for about 2 days in a first cell culture medium to obtain iPSC-derived cell intermediates, wherein said first cell culture medium is StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the iPSC-derived cell intermediates for an additional about 2 days in a second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM;
e) culturing the iPSC-derived cell intermediates for an additional about 2 days in a third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the iPSC-derived cell intermediates for an additional about 2 days to about 4 days in a fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
g) after about 8 days of redifferentiation, collecting non-adherent iPSC-derived cell intermediates from the fourth cell culture medium and adding them back to the culture in the fourth cell culture medium;
h) harvesting redifferentiated iHSCs at day 10 of culture in two different cellular fractions, a non-adherent cellular fraction and an adherent cellular fraction, wherein the redifferentiated iHSCs are harvested in a fifth cell culture medium, wherein the fifth cell culture medium comprises Advanced DMEM/F12 medium and StemFit® For Differentiation medium in about a 4:1 ratio, and further comprises L-glutamine and optionally penicillin/streptomycin, wherein the fifth cell culture medium does not comprise cytokines or growth factors;
i) harvesting non-adherent iHSCs by centrifugation, wherein said harvesting optionally does not include further culturing;
j) harvesting adherent iHSCs by dispersing cells, optionally resuspending the cells obtained from dispersion, followed by passing through a first cell strainer and a second cell strainer, wherein the first cell strainer is 70 μm mesh size and the second cell strainer is 40 μm mesh size;
k) determining the purity of the iHSCs, said purity defined as CD34+ and lineage marker-negative;
l) not introducing serum in any of steps a-k;
m) not introducing any additional cells, including feeder cells, in any of steps a-1; or
n) obtaining de novo generated iHSCs.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method comprising: a) seeding the iPSCs at about 0.4×10⁶ cells/well to about 1.2×10⁶ cells/well of a six-well ultra low adhesion tissue culture vessel or an equivalent thereof; b) culturing the iPSCs for up to about 48 hours with agitation to generate embryoid bodies (EBs); c) transferring the EBs to a tissue culture treated culture vessel and initiating redifferentiation of the EBs for about 10 days, wherein the redifferentiation culture vessel comprises EBs and cells in suspension; d) obtaining the EBs and cells in suspension; and e) optionally, treating the EBs and cells in suspension to obtain a single cell suspension.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method comprising: a) seeding the iPSCs at about 0.4×10⁶ cells/well to about 1.2×10⁶ cells/well of a six-well ultra low adhesion tissue culture vessel or an equivalent thereof; b) culturing the iPSCs for up to about 48 hours with agitation to generate embryoid bodies (EBs); c) transferring the EBs to a tissue culture treated culture vessel and initiating redifferentiation of the EBs for about 10 days, wherein the tissue culture treated culture vessel comprises EBs and cells in suspension; d) obtaining the EBs and cells in suspension; e) optionally, treating the EBs and cells in suspension to obtain a single cell suspension; and f) providing CD34+ and lineage marker-negative iHSCs.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method comprising: a) seeding the iPSCs at about 0.4×10⁶ cells/well to about 1.2×10⁶ cells/well of a six-well ultra low adhesion tissue culture vessel or an equivalent thereof; b) culturing the iPSCs for up to about 48 hours with agitation to generate embryoid bodies (EBs); c) transferring the EBs to a tissue culture treated culture vessel and initiating redifferentiation of the EBs for about 10 days, wherein the tissue culture treated culture vessel comprises EBs and cells in suspension; d) obtaining the EBs and cells in suspension; e) optionally, treating the EBs and cells in suspension to obtain a single cell suspension; and f) obtaining CD34+, and CD3−, and/or CD19−, and/or CD14−, and/or CD11b−, and/or CD11c−, and/or CD56−, and/or CD20−, and/or CD16−, and/or CD2−, and/or CD235a− iHSCs.

In an aspect, the invention provides one or more cells obtained from a method of de novo generation of hematopoietic stem cells (iHSCs) derived from human γδ T cell derived induced pluripotent stem cells (iPSCs) under serum-free, feeder-free culture conditions, said method comprising:
i) obtaining one or more human γδ T cell derived iPSCs, and
wherein the method further comprises one or more of the following steps:
   a) seeding the iPSCs at about 0.6×10⁶ cells/well of a six-well ultra low adhesion tissue culture vessel or an equivalent thereof;
   b) maintaining the iPSCs in culture conditions for up to 48 hours with agitation to generate embryoid bodies (EBs);
   c) transferring the EBs from the ultra low adhesion tissue culture vessel to a tissue culture treated tissue culture vessel and redifferentiating the EBs by culturing the EBs for about 2 days in a first cell culture medium, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
   d) culturing EBs and cells in suspension for an additional about 2 days in a second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
   e) culturing the EBs and cells in suspension for an additional about 2 days in a third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
   f) culturing the EBs and cells in suspension for an additional about 2 days to about 4 days in a fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
   g) after about 10 days of redifferentiation, harvesting the EBs and cells in suspension by centrifugation, thereby forming pelleted EBs and cells in suspension;
   h) resuspending the pelleted EBs and cells in suspension in a volume of about 20 mg/mL collagenase type II and incubating the EBs and cells in suspension in the collagenase type II;
   i) adding TrypLE to the EBs and the cells in suspension and incubating the EBs and the cells in suspension to obtain a single cell suspension,
   j) mixing the single cell suspension;
   k) adding a fifth cell culture medium to the cell suspension, wherein said fifth cell culture medium comprises Advanced DMEM/F12 medium and StemFit® For Differentiation medium in a ratio of about a 4:1 ratio, and further comprises L-glutamine and optionally penicillin/streptomycin, wherein the fifth cell culture medium does not comprise cytokines or growth factors,
   l) centrifuging the cell suspension to form a cell pellet and resuspending the cell pellet in the fifth cell culture medium to form a second cell suspension;
   m) passing the second cell suspension through a first cell strainer and a second cell strainer to obtain iHSCs, wherein the first cell strainer is 70 μm mesh size and the second cell strainer is 40 μm mesh size;
   n) determining the purity of the iHSCs, said purity defined as CD34+ and lineage marker-negative;
   o) not introducing serum in any of steps a-n;
   p) not introducing any additional cells, including feeder cells or stromal cells, in any of steps a-1; or
   q) obtaining de novo generated iHSCs.

In any of the embodiments herein, the resulting iHSCs have high purity, viability, fold expansion, and potential to redifferentiate into immune effectors, including iPSC-derived γδ (iγδ) T cells.

In another aspect, the invention provides a composition comprising a cell produced by any of the methods described herein.

In another aspect, the invention provides a use of cells obtained by any of the methods described herein, in preparation of cells for treating a pathology, disease(s), in preparation of lymphocytes, in a bioreactor, in tissue engineering or in vitro drug screening for diseases.

In another aspect, the invention provides a system, wherein any of the methods described herein are performed by hand or with automated robotic assistance or a combination thereof.

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said method comprising: seeding human γδ T cell derived iPSCs in a tissue culture vessel wherein the iPSCs are maintained for up to about 6 days; initiating redifferentiation of the iPSCs to iHSCs by culturing in the tissue culture vessel for about 2 days to obtain iPSC-derived cell intermediates; culturing the iPSC-derived cell intermediates for about 8 days; collecting non-adherent iPSC-derived cell intermediates and adding them back to the culture; and harvesting redifferentiated iHSCs at about day 10 of culture into two different cellular fractions, a non-adherent cellular fraction and an adherent cellular fraction.

In some embodiments, the method further comprises initiating redifferentiation of the iPSCs in a composition comprising basic fibroblast growth factor (bFGF), a glycogen synthase kinase inhibitor, a bone morphogenetic protein (BMP4), and vascular endothelial growth factor (VEGF).

In some embodiments, the method further comprises culturing the iPSC-derived cell intermediates for about 2 days in a composition comprising effective amounts of L-glutamine, an inhibitor of the Activin/BMP/TGFβ pathway), VEGF, bFGF, and, optionally, a stem cell factor (SCF) and/or antibiotic(s).

In some embodiments, the methods further comprise culturing the iPSC-derived cell intermediates for about 2 days in a composition comprising L-glutamine, SCF, VEGF, FMS-like tyrosine kinase 3 ligand (Flt3L), erythropoietin (EPO), interleukin-3 (IL-3), interleukin-6 (IL-6), and optionally antibiotic(s).

In some embodiments, the methods further comprise culturing the iPSC-derived cell intermediates for about 2 days in a composition comprising L-glutamine, SCF, IL-6, EPO, and optionally antibiotic(s).

In some embodiments, the methods further comprise culturing redifferentiated iHSCs for about 2 days in a composition comprising L-glutamine and optionally antibiotic(s), wherein the composition does not comprise cytokines or growth factors.

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said system comprising one or more components capable of performing a method comprising:
obtaining at least one human γδ T cell derived iPSC, and
wherein the method further comprises one or more of the following steps:
a) seeding the iPSCs at about 1.7×10³ cells/well to about 2.5×10³ cells per well, preferably about 2×10³ cells/well of a six-well tissue culture vessel or an equivalent thereof;
b) maintaining the iPSCs as colonies under culture conditions for up to 6 days;
c) initiating redifferentiation of the iPSCs to iHSCs by culturing the iPSCs for about 2 days in a first cell culture medium to obtain the iPSC-derived cell intermediates, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the iPSC-derived cell intermediates for an additional about 2 days in a second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
e) culturing the iPSC-derived cell intermediates for an additional about 2 days in a third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the iPSC-derived cell intermediates for an additional about 2 days to about 4 days in a fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
g) after about 8 days of redifferentiation, collecting non-adherent the iPSC-derived cell intermediates from the fourth cell culture medium and adding them back to the culture in the fourth cell culture medium;
h) harvesting redifferentiated iHSCs at day 10 of culture in two different cellular fractions, a non-adherent cellular fraction and an adherent cellular fraction, wherein the redifferentiated iHSCs are harvested in a fifth cell culture medium, wherein the fifth cell culture medium comprises Advanced DMEM/F12 medium and StemFit® For Differentiation medium in about a 3.9 to 0.9 ratio to about a 4.1 to 1.1 ratio, and further comprises L-glutamine and optionally penicillin/streptomycin, wherein the fifth cell culture medium does not comprise cytokines or growth factors;
i) harvesting non-adherent iHSCs by centrifugation, wherein said harvesting optionally does not include further culturing;
j) harvesting adherent iHSCs by dispersing cells, optionally resuspending the cells obtained from dispersion, followed by passing through a first cell strainer and a second cell strainer, wherein the first cell strainer is 70 μm mesh size and the second cell strainer is 40 μm mesh size;
k) determining the purity of the iHSCs, said purity defined as CD34+ and lineage marker-negative;
l) not introducing serum in any of steps a-k;
m) not introducing any additional cells, including feeder cells, in any of steps a-1; or
n) obtaining de novo generated (iHSCs).

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said method comprising: a) seeding the iPSCs at about 0.4×10⁶ cells/well to about 1.2×10⁶ cells/well of a six-well ultra low adhesion tissue culture vessel or an equivalent thereof; b) culturing the iPSCs for up to about 48 hours with agitation to generate embryoid bodies (EBs); c) transferring the EBs to a tissue culture treated culture vessel and initiating redifferentiation of the EBs for about 10 days, wherein the tissue culture treated culture vessel comprises EBs and cells in suspension; d) obtaining the EBs and cells in suspension; and e) optionally, treating the EBs and cells in suspension to obtain a single cell suspension.

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said method comprising: a) seeding the iPSCs at about 0.4×10⁶ cells/well to about 1.2×10⁶ cells/well of a six-well ultra low adhesion tissue culture vessel or an equivalent thereof; b) culturing the iPSCs for up to about 48 hours with agitation to generate embryoid bodies (EBs); c) transferring the EBs to a tissue culture treated culture vessel and initiating redifferentiation of the EBs for about 10 days, wherein the tissue culture treated culture vessel comprises EBs and cells in suspension; d) obtaining the EBs and cells in suspension; e) optionally, treating the EBs and cells in suspension to obtain a single cell suspension; and f) providing CD34+ and lineage marker-negative iHSCs.

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said method comprising: a) seeding the iPSCs at about 0.4×10⁶ cells/well to about 1.2×10⁶ cells/well of a six-well ultra low adhesion tissue culture vessel or an equivalent thereof; b) culturing the iPSCs for up to about 48 hours with agitation to generate embryoid bodies (EBs); c) transferring the EBs to a tissue culture treated culture vessel and initiating redifferentiation of the EBs for about 10 days, wherein the tissue culture treted culture vessel comprises EBs and cells in suspension; d) obtaining the EBs and cells in suspension; e) optionally, treating the EBs and cells in suspension to obtain a single cell suspension; and f) obtaining CD34+, and CD3−, and/or CD19−, and/or CD14−, and/or CD11b−, and/or CD11c−, and/or CD56−, and/or CD20−, and/or CD16−, and/or CD2−, and/or CD235a− iHSCs.

In an aspect, the invention provides a system for performing a method of de novo generation of hematopoietic stem cells (iHSCs) derived from human γδ T cell derived induced pluripotent stem cells (iPSCs) under serum-free, feeder-free culture conditions, said method comprising:
i) obtaining one or more human γδ T cell derived iPSCs, and
wherein the method further comprises one or more of the following steps:
   a) seeding the iPSCs at about 0.6×10⁶ cells/well of a six-well ultra low adhesion tissue culture vessel or an equivalent thereof;
   b) maintaining the iPSCs in culture conditions for up to 48 hours to generate embryoid bodies (EBs);
   c) transferring the EBs from the ultra low adhesion tissue culture vessel to a tissue culture treated tissue culture vessel and redifferentiating the EBs by culturing the EBs for about 2 days in a first cell culture medium, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
   d) culturing EBs and cells in suspension for an additional about 2 days in a second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
   e) culturing the EBs and cells in suspension for an additional about 2 days in a third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
   f) culturing the EBs and cells in suspension for an additional about 2 days to about 4 days in a fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
   g) after about 10 days of redifferentiation, harvesting the EBs and cells in suspension by centrifugation, thereby forming pelleted EBs and cells in suspension;
   h) resuspending the pelleted EBs and cells in suspension in a volume of about 20 mg/mL collagenase type II and incubating the EBs and cells in suspension in the collagenase type II;
   i) adding TrypLE to the EBs and cells in suspension and incubating the EBs and cells in suspension to obtain a single cell suspension,
   j) mixing the single cell suspension;
   k) adding a fifth cell culture medium to the cell suspension, wherein said fifth cell culture medium comprises Advanced DMEM/F12 medium and StemFit® For Differentiation medium in a ratio of about a 4:1 ratio, and further comprises L-glutamine and optionally penicillin/streptomycin, wherein the fifth cell culture medium does not comprise cytokines or growth factors,
   l) centrifuging the cell suspension to form a cell pellet and resuspending the cell pellet in the fifth cell culture medium to form a second cell suspension;
   m) passing the second cell suspension through a first cell strainer and a second cell strainer to obtain iHSCs, wherein the first cell strainer is 70 μm mesh size and the second cell strainer is 40 μm mesh size;
   n) determining the purity of the iHSCs in the single cell suspension, said purity defined as CD34+ and lineage marker-negative;
   o) not introducing serum in any of steps a-n;
   p) not introducing any additional cells, including feeder cells or stromal cells, in any of steps a-o; or
   q) obtaining de novo generated iHSCs.

In some embodiments, the step of determining purity of the iHSCs comprises assaying for one or more, optionally all, of the lineage markers CD3, CD19, CD14, CD11b, CD11c, CD56, CD20, CD16, CD2, and CD235a via flow cytometry or an equivalent thereof.

In some embodiments, the lineage markers comprise one or more of CD3, CD19, CD14, CD11b, CD11c, CD56, CD20, CD16, CD2, and CD235a.

In some embodiments, the step of mixing the single cell suspension comprises gently dispersing the cells using a pipet or equivalent thereof.

In some embodiments, the system is automated or semi-automated.

In some embodiments, the system comprises one or more software packages, the software package(s) operating and scheduling operation of the system.

In some embodiments, the software package(s) are customized and or customizable for desired applications and are, optionally, menu-driven.

In some embodiments, the automation comprises cell visualization, plate handling, plate coating, seeding, extraction, addition, cell feeding, incubation assays and or sampling.

In some embodiments, the system comprises one or more incubators on line.

In some embodiments, the system comprises electronic humidity controls, a HEPA filter system, a carousel, said carousel comprising programmable stepping, oscillation cycles and or a two-way communication interface or any combination thereof.

In some embodiments, medium is added or removed or supplemented without disturbing or contaminating cells.

In some embodiments, the system comprises one or more computer operated and controlled robotic arms.

### BRIEF DESCRIPTION OF THE DRAWINGS

FIG. 1A-1D shows a nonlimiting exemplary automated system format for performing any of the methods described herein. FIG. 1A shows a front view of the system, FIG. 1B shows a top view of the system of FIG. 1A, and FIGS. 1C (an exemplary, vertically stackable view) and 1D (an exemplary, top down view of six systems) show side views of the system in which the components are stacked on top of each other (FIG. 1C) or are side by side (FIG. 1D) and which are each stationary or mobile.

FIG. 2 shows a flow diagram of a nonlimiting exemplary SFFF 2D iHSC redifferentiation protocol. On Days 6-10, nonadherent cells that are harvested with the medium change are added back to the culture.

FIG. 3 shows a flow diagram of a nonlimiting exemplary SFFF 3D/2D iHSC redifferentiation protocol.

FIG. 4 shows a schematic of a nonlimiting exemplary SFFF 2D iHSC redifferentiation protocol.

FIGS. 5A-5B show cells obtained from a nonlimiting exemplary SFFF 2D iHSC redifferentiation protocol (FIG. 5A) and from a nonlimiting exemplary SFFF 3D/2D iHSC redifferentiation protocol (FIG. 5B) according to an embodiment herein.

FIGS. 6A-6C show effects of a nonlimiting exemplary SFFF 2D iHSC redifferentiation protocol according to an embodiment herein. FIG. 6A shows a time course of iHSC redifferentiation from three iPSC clones (top row, Clone 1; middle row, Clone 2; bottom row, Clone 3) over a 10-day period. FIG. 6B shows the effects of the nonlimiting exemplary SFFF 2D iHSC redifferentiation protocol on purity (right panel) and fold expansion (left panel) of redifferentiated iHSCs obtained from the same three iPSC clones as shown in FIG. 6A, as well as for adherent and non-adherent fractions. FIG. 6C shows the effects of the nonlimiting exemplary SFFF 2D iHSC redifferentiation protocol on purity (right panel) and fold expansion (left panel) of iγδ T cells obtained from iHSCs derived from the same three iPSC clones, as well as for adherent and non-adherent fractions.

FIG. 7A-7B show results of a nonlimiting exemplary 3D/2D protocol disclosed herein. FIG. 7A shows the results of the nonlimiting exemplary SFFF 3D/2D iHSC redifferentiation protocol on iHSC purity (right panel) and fold expansion (left panel) for iPSC Clone 1. FIG. 7B shows the results of the nonlimiting exemplary SFFF 3D/2D iHSC redifferentiation protocol on iγδ T cell purity (right panel) and fold expansion (left panel) for Clone 1.

FIG. 8A-8MM show microscopic images of cells obtained at different stages of the nonlimiting exemplary 2D and 2D/3D protocols disclosed herein.

### DETAILED DESCRIPTION

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of up to ±10% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

The term “subject” refers to human and non-human animals, including all vertebrates, e.g., mammals and non-mammals, such as non-human primates, mice, rabbits, sheep, dogs, cats, horses, cows, chickens, amphibians, and reptiles. In many embodiments of the described methods, the subject is a human.

The term “induced pluripotent stem cell” (hereinafter “iPSC”) means a stem cell that is established by introducing reprogramming factors into a somatic cell, has pluripotency permitting differentiation into many cell types present in living organisms, and also has proliferative (i.e., self-renewal) capacity. It encompasses any cell that can be redifferentiated into a hematopoietic stem cell (HSC) to be used in the present invention. The iPSC is preferably derived from a primate (e.g., monkey, orangutan, chimpanzee, human), more preferably human.

The term “iHSC” refers to hematopoietic stem cells that are derived from iPSCs.

The term “multicellular body” includes but is not limited to embryoid bodies.

The embodiments described herein are not limited to particular methods, reagents, compounds, compositions, or biological systems, which can, of course, vary.

Methods of Generating iHSCs

γδ T cells were selected as the iPSC cell of origin because it is known that the epigenetic and transcriptional memory of the starting cell influences and enhances iPSC redifferentiation potential to that cell type. An optimized two-step, two-dimensional (2D), research-scale, SFFF protocol was used to redifferentiate γδ T-iPSCs into iHSCs and then into iγδ T cells. Purity and fold expansion were determined for each redifferentiation step, and the cells were examined under the microscope to evaluate morphological characteristics for the redifferentiation of iPSCs to iHSCs. Success criteria were set for the purity and fold expansion of redifferentiated iHSCs and iγδ T cells based on results from a 2D redifferentiation protocol and from a 3D/2D redifferentiation protocol. Optimizing the protocol for the redifferentiation of iPSCs to iHSCs resulted in their increased purity and fold expansion, exceeding the success criteria set for this redifferentiation step. Protocol optimization of the iHSC redifferentiation step also increased the purity and fold expansion of iγδ T cells generated in the second redifferentiation step. Notably, enhanced T cell redifferentiation potential was observed for three γδ T-iPSC clones (two of which are subclones), demonstrating the efficiency and validity of the optimized protocol for the redifferentiation of iPSCs to iHSCs.

In an aspect, the present invention provides a method of de novo generation of hematopoietic stem cells (iHSCs) derived from human γδ T cell derived induced pluripotent stem cells (iPSCs) comprising one or more, optionally all, of the following steps: (i) maintaining iPSCs prior to initiating redifferentiation; (ii) redifferentiation of the iPSCs to iHSCs; and (iii) differentiation of iHSCs to iγδ T cells.

In another aspect, the present invention provides a method of de novo generation of hematopoietic stem cells derived from human γδ T cell derived induced pluripotent stem cells (iPSCs) under serum-free and feeder-free culture conditions, said method comprising: (i) seeding human γδ T cell derived iPSCs in a tissue culture vessel wherein the iPSCs are maintained for up to about 6 days; (ii) initiating redifferentiation of the iPSCs to iHSCs by culturing in the tissue culture vessel for about 2 days to obtain iPSC-derived cell intermediates; (iii) culturing the iPSC-derived cell intermediates for about 8 days; (iv) collecting non-adherent iPSC-derived cell intermediates and adding them back to the culture; and (v) harvesting redifferentiated iHSCs at about day 10 of culture into two different cellular fractions, a non-adherent cellular fraction and an adherent cellular fraction.

In some embodiments, the methods further comprise the step of initiating redifferentiation of the iPSCs in a composition comprising basic fibroblast growth factor (bFGF), a glycogen synthase kinase inhibitor, a bone morphogenetic protein (BMP4), and vascular endothelial growth factor (VEGF).

In some embodiments, the methods further comprise culturing the iPSC-derived cell intermediates for about 2 days in a composition comprising effective amounts of L-glutamine, an inhibitor of the Activin/BMP/TGFβ pathway, VEGF, bFGF, and, optionally, a stem cell factor (SCF) and/or antibiotic(s).

In some embodiments, the methods further comprise culturing the iPSC-derived cell intermediates for about 2 days in a composition comprising L-glutamine, SCF, VEGF, FMS-like tyrosine kinase 3 ligand (Flt3L), erythropoietin (EPO), interleukin-3 (IL-3), interleukin-6 (IL-6), and optionally antibiotic(s).

In some embodiments, the methods further comprise culturing the iPSC-derived cell intermediates for about 2 days in a composition comprising L-glutamine, SCF, IL-6, EPO, and optionally antibiotic(s).

In some embodiments, the methods further comprise culturing redifferentiated iHSCs for about 2 days in a composition comprising L-glutamine and optionally antibiotic(s), wherein the composition does not comprise cytokines or growth factors.

In another aspect, the present invention provides a method of de novo generation of hematopoietic stem cells (iHSCs) derived from human γδ T cell derived induced pluripotent stem cells (iPSCs) under serum-free and feeder-free culture conditions, said method comprising:
i) obtaining one or more human γδ T cell derived iPSCs, and
wherein the method further comprises one or more of the following steps:
a) seeding iPSCs at about 1.7×10³ cells/well to about 2.5×10³ cells per well, preferably about 2×10³ cells/well of a six-well tissue culture vessel or an equivalent thereof;
b) maintaining the iPSCs under culture conditions for about 5.5 to about 6.5 days, preferably up to 6 days;
c) initiating redifferentiation of the iPSCs into iHSCs by culturing the iPSCs for about 1.8 days to about 2.2 days, preferably about 2 days in a first cell culture medium to obtain iPSC-derived cell intermediates, wherein said first cell culture medium comprises StemFit® Basic 04 medium supplemented with basic fibroblast growth factor (bFGF) to a final concentration of about 90 ng/mL to about 110 ng/mL bFGF, preferably about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the iPSC-derived cell intermediates for an additional about 1.8 days to about 2.2 days, preferably about 2 days in a second cell culture medium, wherein said second cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
e) culturing the iPSC-derived cell intermediates for an additional about 1.8 days to about 2.2 days, preferably about 2 days in a third cell culture medium, wherein said third cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the iPSC-derived cell intermediates for an additional about 1.8 days to about 4.2 days, preferably about 2 days to about 4 days in a fourth cell culture medium, wherein said fourth cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SCF, IL-6, EPO, and optionally penicillin/streptomycin;
g) after about 7.8 days to about 8.2 days, preferably 8 days, of redifferentiation, collecting non-adherent iPSC-derived cell intermediates from the fourth cell culture medium and adding them back to the culture in the fourth cell culture medium;
h) harvesting redifferentiated iHSCs at day 10 of culture in two different cellular fractions, a non-adherent cellular fraction and an adherent cellular fraction, wherein the redifferentiated iHSCs are harvested in a fifth cell culture medium, wherein the fifth cell culture medium comprises Advanced DMEM/F12 medium and StemFit® For Differentiation medium in about a 3.9 to 0.9 ratio to about a 4.1-1.1 ratio, preferably about a 4:1 ratio, and further comprises L-glutamine and optionally penicillin/streptomycin, wherein the fifth cell culture medium does not comprise cytokines or growth factors;
i) harvesting non-adherent iHSCs by centrifugation, wherein said harvesting optionally does not include further culturing;
j) harvesting adherent iHSCs by dispersing cells, optionally resuspending the cells obtained from dispersion, followed by passing through a first cell strainer and a second cell strainer, wherein the first cell strainer is 70 μm mesh size and the second cell strainer is 40 μm mesh size;
k) determining the purity of the iHSCs, said purity defined as CD34+ and lineage marker-negative;
l) not introducing serum in any of steps a-k;
n) not introducing any additional cells, including feeder cells, in any of steps a-m; or
o) obtaining de novo generated iHSCs.

In an aspect, the present invention comprises a method of de novo generation of hematopoietic stem cells (iHSCs) derived from human γδ T cell derived induced pluripotent stem cell (iPSC) under serum-free and feeder-free culture conditions, said method comprising: a) seeding the iPSCs at about 0.4×10⁶ cells/well to about 1.2×10⁶ cells/well of a six-well ultra low adhesion tissue culture vessel or an equivalent thereof; b) culturing the iPSCs for up to about 48 hours with agitation to generate embryoid bodies (EBs); c) transferring the EBs to a tissue culture treated culture vessel and initiating redifferentiation of the EBs for about 10 days, wherein the tissue culture treated culture vessel comprises EBs and cells in suspension; d) obtaining the EBs and cells in suspension; and e) optionally, treating the EBs and cells in suspension to obtain a single cell suspension.

In some embodiments, the present invention further comprises obtaining CD34+ and lineage marker-negative iHSCs.

In some embodiments, the present invention further comprises obtaining iHSCs that are CD34+, and CD3−, and/or CD19−, and/or CD14−, and/or CD11b−, and/or CD56−, and/or CD20−, and/or CD16−, and/or CD2−, and/or CD235a− cells.

In an aspect, the present invention provides a method of de novo generation of hematopoietic stem cells (iHSCs) derived from human γδ T cell derived induced pluripotent stem cells (iPSCs) under serum-free, feeder-free culture conditions, said method comprising:
i) obtaining one or more human γδ T cell derived iPSCs, and
wherein the method further comprises one or more of the following steps:
a) seeding the iPSCs at about 0.4×10⁶ cells/well to about 1.2×10⁶ cells/well, preferably about 0.6×10⁶ cells/well, of a six-well ultra low adhesion tissue culture vessel or an equivalent thereof;
b) maintaining the iPSCs in culture conditions for up to 48 hours with agitation to generate embryoid bodies (EBs);
c) transferring the EBs from the ultra low adhesion tissue culture vessel to a tissue culture treated tissue culture vessel and redifferentiating the EBs by culturing the EBs for about 2 days in a first cell culture medium, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing EBs and cells in suspension for an additional about 2 days in a second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
e) culturing the EBs and cells in suspension for an additional about 2 days in a third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the EBs and cells in suspension for an additional about 2 days to about 4 days in a fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
g) after about 10 days of redifferentiation, harvesting the EBs and cells in suspension by centrifugation, thereby forming pelleted EBs and cells in suspension;
h) resuspending the pelleted EBs and cells in suspension in a volume of about 20 mg/mL collagenase type II and incubating the EBs and cells in suspension in the collagenase type II;
i) adding TrypLE to the EBs and the cells in suspension and incubating the EBs and the cells in suspension to obtain a single cell suspension,
j) mixing the single cell suspension;
k) adding a fifth cell culture medium to the cell suspension, wherein said fifth cell culture medium comprises Advanced DMEM/F12 medium and StemFit® For Differentiation medium in a ratio of about a 4:1 ratio, and further comprises L-glutamine and optionally penicillin/streptomycin, wherein the fifth cell culture medium does not comprise cytokines or growth factors,
l) centrifuging the cell suspension to form a cell pellet and resuspending the cell pellet in the fifth cell culture medium to form a second cell suspension;
m) passing the second cell suspension through a first cell strainer and a second cell strainer to obtain iHSCs, wherein the first cell strainer is 70 μm mesh size and the second cell strainer is 40 μm mesh size;
n) determining the purity of the iHSCs, said purity defined as CD34+ and lineage marker-negative;
o) not introducing serum in any of steps a-n;
p) not introducing any additional cells, including feeder cells or stromal cells, in any of steps a-o; or
q) obtaining de novo generated iHSCs.

In an aspect, the invention provides a method of de novo generation of hematopoietic stem cells (iHSCs) derived from human γδ T cell derived induced pluripotent stem cells (iPSCs) under serum-free, feeder-free culture conditions, said method comprising: a) a step for performing a function of initiating redifferentiation of the iPSCs to iHSCS by culturing in a tissue culture vessel for about 2 days to obtain iPSC-derived cell intermediates; (b) a step for performing a function of culturing the iPSC-derived cell intermediates for about 8 days; (c) a step for performing a function of collecting non-adherent iPSC-derived cell intermediates and adding them back to the culture; and (d) a step for performing a function of harvesting redifferentiated iHSCs at about day 10 of culture into two different cellular fractions, a non-adherent cellular fraction and an adherent cellular fraction.

In an aspect, the invention provides a method of de novo generation of hematopoietic stem cells (iHSCs) derived from human γδ T cell derived induced pluripotent stem cells (iPSCs) under serum-free and feeder-free culture conditions, said method comprising: a) a step for performing a function of seeding the iPSCs; b) a step for performing a function of culturing the iPSCs for up to about 48 hours with agitation to generate embryoid bodies (EBs); c) a step for performing a function of transferring the EBs to a tissue culture treated culture vessel and initiating redifferentiation of the EBs for about 10 days, wherein the tissue culture treated culture vessel comprises EBs and cells in suspension; d) a step for performing a function of obtaining the EBs and cells in suspension; and e) optionally, a step for performing a function of treating the EBs and cells in suspension to obtain a single cell suspension.

In an aspect, the invention provides a method of de novo generation of hematopoietic stem cells (iHSCs) derived from human γδ T cell derived induced pluripotent stem cells (iPSCs) under serum-free and feeder-free culture conditions, said method comprising: a) a step for performing a function of seeding the iPSCs; b) a step for performing a function of culturing the iPSCs for up to about 48 hours with agitation to generate embryoid bodies (EBs); c) a step for performing a function of transferring the EBs to a tissue culture treated culture vessel and initiating redifferentiation of the EBs for about 10 days, wherein the redifferentiation culture vessel comprises EBs and cells in suspension; d) a step for performing a function of culturing the EBs and cells in suspension for about 2 to about 4 days; e) a step for performing a function of obtaining the EBs and cells in suspension; f) a step for performing a function of optionally, treating the EBs and cells in suspension to obtain a single cell suspension; and g) a step for performing a function of providing CD34+ and lineage marker-negative iHSCs.

In an aspect, the invention provides a method of de novo generation of hematopoietic stem cells (iHSCs) derived from human γδ T cell derived induced pluripotent stem cells (iPSCs) under serum-free and feeder-free culture conditions, said method comprising: a) a step for performing a function of seeding the iPSCs; b) a step for performing a function of culturing the iPSCs for up to about 48 hours with agitation to generate embryoid bodies (EBs); c) a step for performing a function of transferring the EBs to a tissue culture treated culture vessel and initiating redifferentiation of the EBs for about 10 days, wherein the redifferentiation culture vessel comprises EBs and cells in suspension; d) a step for performing a function of obtaining the EBs and cells in suspension; e) optionally, a step for performing a function of treating the EBs and cells in suspension to obtain a single cell suspension; and f) a step for performing a function of obtaining CD34+, and CD3−, and/or CD19−, and/or CD14−, and/or CD11b−, and/or CD11c−, and/or CD56−, and/or CD20−, and/or CD16−, and/or CD2−, and/or CD235a− iHSCs.

In some embodiments of any of the methods disclosed herein, the cells are cultured under normoxic conditions.

In some embodiments of any of the methods disclosed herein, the method further comprises further purifying and/or isolating the iHSCs.

In some embodiments of any of the methods disclosed herein, the obtained iHSCs are isolated differentiated cells or are capable of being further purified and/or isolated.

In some embodiments of any of the methods disclosed herein, the step of determining the purity of the iHSCs comprises assaying for one or more, optionally all, of the lineage markers CD3, CD19, CD14, CD11b, CD11c, CD56, CD20, CD16, CD2, and CD235a via flow cytometry or an equivalent thereof.

In some embodiments of any of the methods disclosed herein, the step of mixing the single cell suspension comprises gently dispersing the cells using a pipet or equivalent thereof.

None of the methods described herein require the use of serum or additional cells, such as feeder or stromal cells, in any of the steps. Each step is discussed in more detail below.

Biology, bioengineering generally and biologic technologies, including biologic medicines are theoretically amenable to fundamental bioprocesses improvements which could potentially consolidate research and development and advance industrial progress, including in areas such as medical therapies, diagnostic and immunization procedures, agriculture, food production, biofuel production, and environmental solutions. See, e.g., Alejandro Barragán-Ocaña et al., Biotechnology and Bioprocesses: Their Contribution to Sustainability, Processes 2020, vol. 8, p. 436; and K. V. Boodhoo et al., Bioprocess intensification: A route to efficient and sustainable biocatalytic transformations for the future, Chemical Engineering & Processing: Process Intensification 172 (2022) 108793, each of which is incorporated by reference in its entirety as if completely set forth herein. Even so, only initial steps have identified scientific and technological bioprocess trajectories within the framework of sustainability. One study highlighted activity and confirmed the field as positioned as an emerging knowledge area aimed at, for example, improving economic development, environmental protection, and social welfare. With these considerations in mind, the inventors of the present invention streamlined and optimized materials and processes to, inter alia, reduce and otherwise minimize reagent and resource usage, reduce overall waste including waste of useful biomass, lower costs, develop systems to recycle and reuse materials and decrease waste generation.

Maintaining iPSCs Prior to Initiating Redifferentiation

The iPSCs are cultured as colonies or multicellular bodies. As is known in the art, iPSC colonies generally exhibit circular colonies, with densely packed cells, higher nucleus to cytoplasmic ratio, well defined edges, and distinct borders.

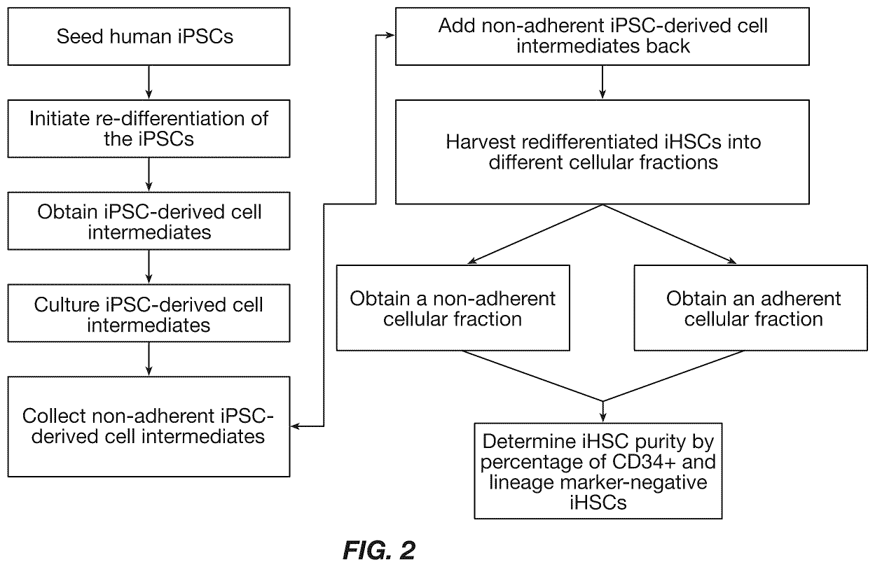

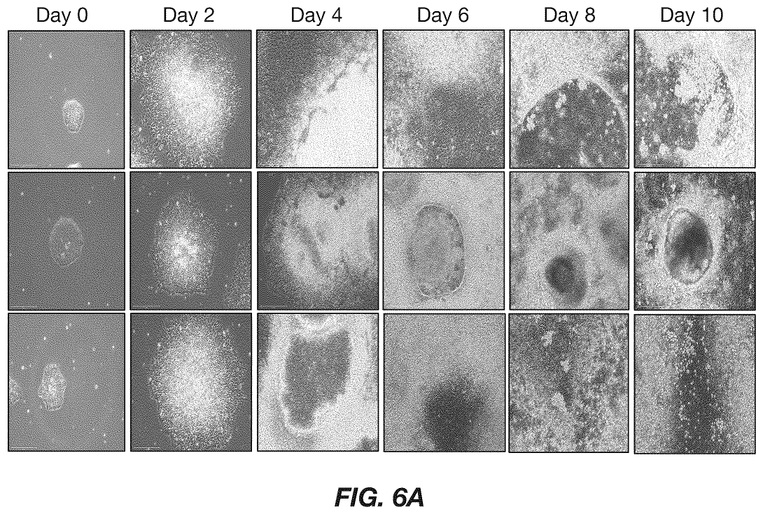

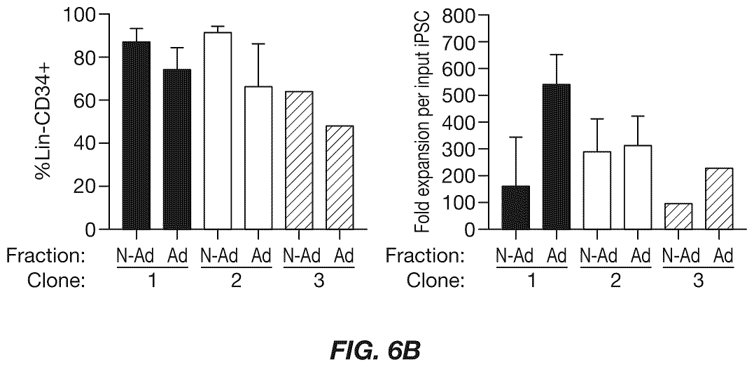

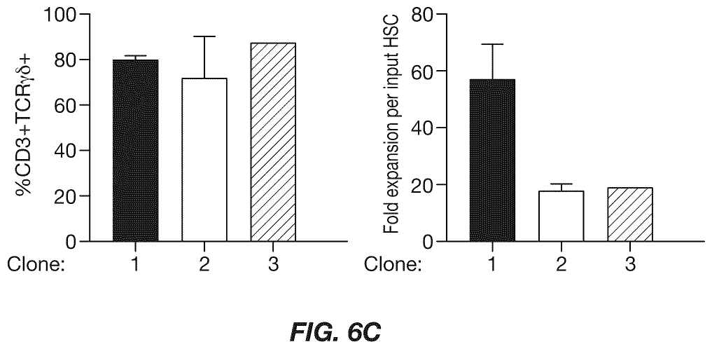

Under nonlimiting exemplary 2D conditions as shown in FIG. 2, the iPSCs are maintained under culture conditions for about 5.5 to about 6.5 days, preferably up to 6 days. Results from redifferentiating the iPSCs into iHSCs, followed by redifferentiating the iHSCs into iγδ T cells as described herein are shown in FIG. 6A-6C.

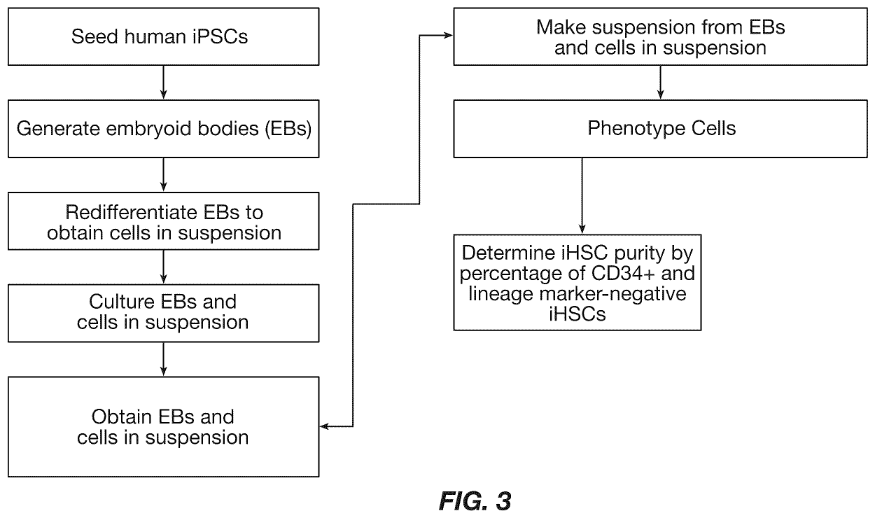

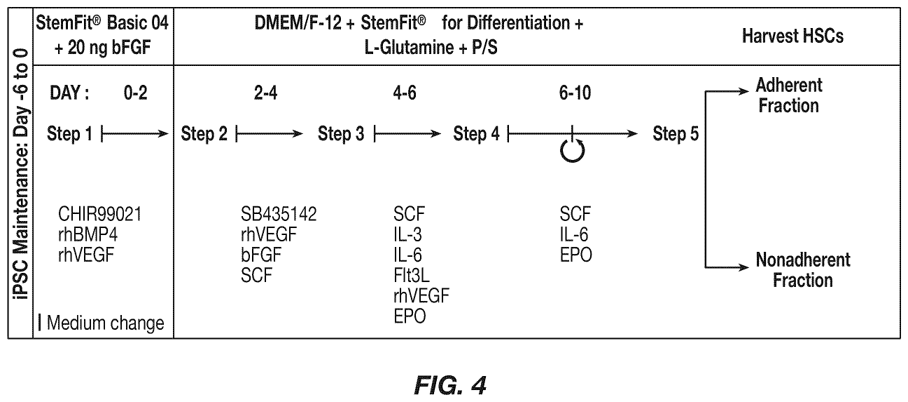

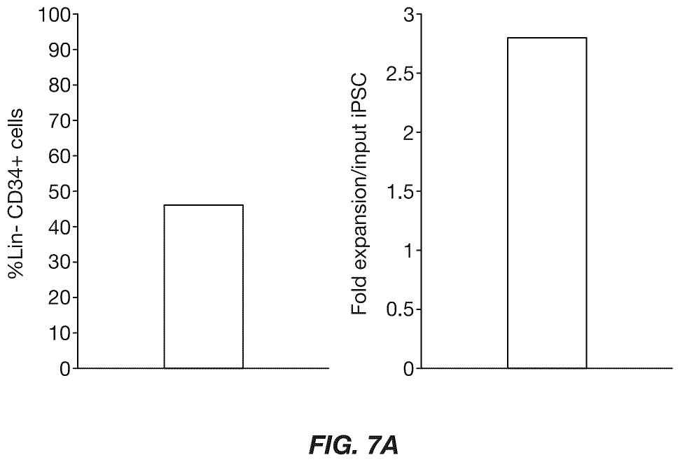

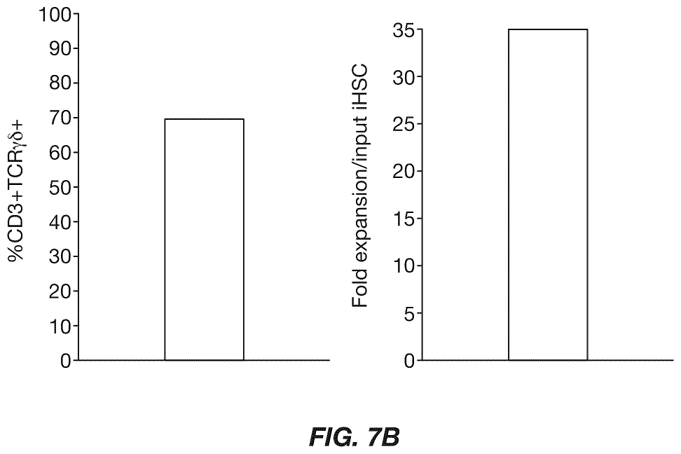

To culture the iPSCs as multicellular bodies, e.g., embryoid bodies in the nonlimiting exemplary 3D/2D protocol as shown in FIG. 3, the iPSCs can be cultured in low attachment tissue culture vessels and can be cultured with agitation. For example, the low attachment tissue culture vessel can be incubated in a shaker incubator/multitron at 37° C., 5% CO₂ with 85% humidity for about 44 hours to about 52 hours, preferably about 48 hours, at 100 RPM. Results from redifferentiating the iPSCs cultured as multicellular bodies into iHSCs, followed by redifferentiating the iHSCs into iγδ T cells as described herein are shown in FIG. 7A-7B.

Optionally, the iPSCs are thawed from a frozen stock, and a desired number of live cells can be utilized in the methods described herein. Cell counting, size, and viability determination can be performed by any method known in the art, e.g., by using a NucleoCounter NC-200. Live cells having a preferred size, e.g., between 10 μm and 25 μm, can be used in the steps described herein.

In a preferred embodiment, the dissociated iPSCs are seeded at a cell density of about 1.7×10³ cells/well to about 2.5×10³ cells/well, preferably about 2×10³ cells/well, for the 2D protocol and at about 0.4×10⁶ cells/well to about 1.2×10⁶ cells/well, preferably about 0.6×10⁶ cells/well, for the 3D/2D protocol. A person of ordinary skill in the art will be able to calculate the number of live cells to seed given the size of the surface area (e.g., well).

After seeding, the iPSCs are maintained in an appropriate tissue or cell culture vessel, such as for example and not limitation, a six-well tissue culture plate. The tissue or cell culture vessels are prepared to receive the iPSCs, optionally by addition of a matrix such as a laminin protein (e.g., iMatrix), and incubation at 37° C., 5% CO₂. In a preferred embodiment for the 2D protocol, the tissue or cell culture vessel is prepared for use by addition of a solution of iMatrix 511 at a concentration of 0.25 μg/cm² per well. Suitable coating methods are known in the art.

In a preferred embodiment for the 3D/2D protocol, the iPSCs can be cultured in low attachment tissue culture vessels and can be cultured with agitation e.g., the low attachment tissue culture vessel can be incubated in a shaker incubator/multitron at 37° C., 5% CO₂ with 85% humidity for about 44 hours to about 52 hours, preferably about 48 hours, at 100 RPM. Such conditions lead to the formation of embryoid bodies (EBs) or multicellular bodies. After the incubation with agitation, the EBs can be transferred from the low attachment tissue culture vessel into a tissue culture treated tissue culture vessel and incubated without agitation.

The iPSCs in the 2D protocol are grown in StemFit® Basic 04 medium further comprising basic fibroblast growth factor (bFGF) at a final concentration of about 90 ng/mL to about 110 ng/mL bFGF, preferably about 100 ng/mL bFGF, for about 5.5 days to about 6.5 days, preferably up to 6 days, at 37° C. with 5% CO₂. For the first 24 hours, the medium contains 10 μM Y-27632 ROCK inhibitor. StemFit® Basic 04 cell culture medium (Ajinomoto) is an animal-origin free, defined medium for human pluripotent stem cell culture. This medium is a complete medium and does not require any other supplements.

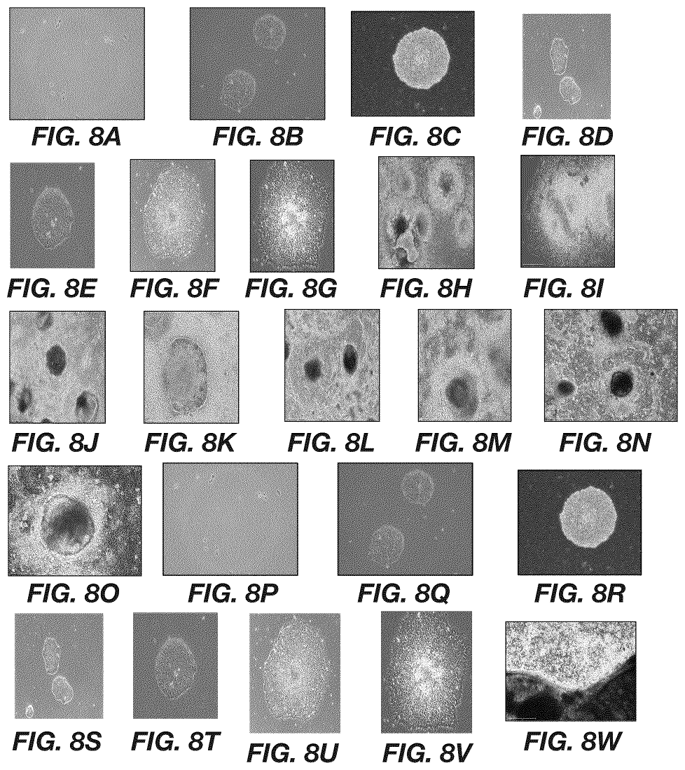

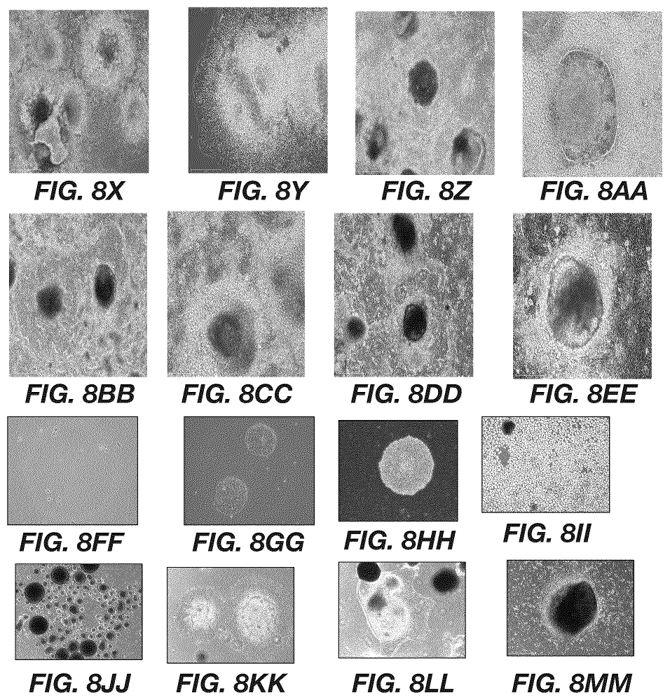

During the maintenance period, the iPSC-derived cell intermediates (of the 2D protocol) or EBs and cells in suspension (of the 3D/2D protocol) can be examined at regular intervals, e.g., daily, for cell morphology as shown in FIG. 8A-8MM. Cell morphology can be determined by microscopy, e.g., inverted phase contrast microscopy. Multicellular (e.g., embryoid) bodies generated using the 3D/2D protocol described herein should be visible at about 48 hours. The multicellular bodies can be collected by centrifugation once formed and do not need to be incubated with agitation after formation. Once the cells are bright and have distinct borders, the colonies can be redifferentiated to iHSCs as discussed herein. The medium can be changed at regular intervals as known in the art.

Redifferentiation of the iPSC-Derived Cell Intermediates or EBs and Cells in Suspension into iHSCs

In the 2D and 3D/2D protocols, the iPSC-derived cell intermediates and/or EBs and cells in suspension, respectively, can be cultured for about 1.8 days to about 2.2 days, preferably about two days, at 37° C., 5% CO₂ in a first culture medium comprising StemFit® Basic 04 medium further comprising basic fibroblast growth factor (bFGF) at a final concentration of about 90 ng/mL to about 110 ng/mL bFGF, preferably about 100 ng/mL bFGF, and one or more of CHIR99021 (glycogen synthase kinase inhibitor), bone morphogenetic protein 4 (BMP4), and recombinant human vascular endothelial growth factor (rhVEGF). No agitation is required for the 3D/2D protocol at this stage. The morphology of the iPSC-derived cell intermediates and/or EBs and cells in suspension, respectively, can be determined at the beginning of the culture period and/or at regular intervals by microscopy, e.g., inverted phase contrast microscopy as shown in FIG. 8A-8MM. Culturing the iPSC-derived cell intermediates and/or EBs and cells in suspension, respectively, in this medium can induce mesoderm formation, which is a prerequisite for hematopoietic lineage specification and differentiation.

After the culture period for mesoderm induction in the 2D and 3D/2D protocols, the iPSC-derived cell intermediates and/or EBs and cells in suspension, respectively, can be collected and subsequently cultured in a second culture medium to begin onset of hematopoietic differentiation, for approximately about 1.8 days to about 2.2 days, preferably approximately about two days, at 37° C. with 5% CO₂. No agitation is required for the 3D/2D protocol at this stage. The second cell culture medium comprises Advanced DMEM/F12 medium (Thermo Fisher Scientific Catalog #12634) and StemFit® For Differentiation medium (Ajinomoto) present in about a 3.9 to 0.9 ratio to about a 4.1-1.1 ratio, preferably about a 4:1 ratio, and further comprises L-glutamine, SB431542 (inhibitor of Activin/BMP/TGFβ pathway), rhVEGF, bFGF, and/or stem cell factor (SCF), and optionally penicillin/streptomycin. SB431542 is diluted from a stock concentration of 5 mM to achieve a final concentration of about 1.5 to 2.5 μM in order to use a minimal amount of the diluent dimethylsulfoxide (DMSO). The morphology of the cells can be determined at the beginning of the culture period and/or at regular intervals by microscopy, e.g., inverted phase contrast microscopy as shown in FIGS. 8A-8MM. Advanced DMEM/F12 medium includes amino acids, vitamins, inorganic salts, proteins, reducing elements, trace elements, dextrose, ethanolamine, hypoxanthine Na, linoleic acid, lipoic acid, phenol red, putrescine 2HCl, sodium pyruvate, and thymidine. StemFit® For Differentiation medium is a chemically defined & animal-origin free (CD-AOF) supplement for differentiation of human embryonic stem (ES) cells and iPSCs.

After the culture period to begin onset of hematopoietic differentiation in the 2D and 3D/2D protocols, the iPSC-derived cell intermediates and/or EBs and cells in suspension, respectively, can be collected and subsequently cultured in a third culture medium to induce hemangioblast formation, for approximately about 1.8 days to about 2.2 days, preferably approximately about two days, at 37° C. with 5% CO₂. No agitation is required for the 3D/2D protocol at this stage. The third culture medium comprises Advanced DMEM/F12 medium and StemFit® For Differentiation medium present in about a 3.9 to 0.9 ratio to about a 4.1-1.1 ratio, preferably about a 4:1 ratio, and further comprises one or more of L-glutamine, SCF, rhVEGF, FMS-like tyrosine kinase 3 ligand (Flt3L), erythropoietin (EPO), interleukin-3 (IL-3), and/or interleukin-6 (IL-6), and optionally penicillin/streptomycin. The EPO is prepared from a stock concentration of 500 IU/mL to a final concentration of 10 IU/mL (e.g., 240 μL EPO stock solution is added to a six-well tissue culture plate). The morphology of the cells can be determined at the beginning of the culture period and/or at regular intervals by microscopy, e.g., inverted phase contrast microscopy as shown in FIGS. 8A-8MM.

After the culture period to induce hemangioblast formation in the 2D and 3D/2D protocols, the iPSC-derived cell intermediates and/or EBs and cells in suspension, respectively, can be collected and subsequently cultured in a fourth culture medium for about 1.8 days to about 4.2 days, preferably about two days to about four days, at 37° C. with 5% CO₂ for the endothelial to hematopoietic transition phase. No agitation is required for the 3D/2D protocol at this stage. The fourth culture medium comprises Advanced DMEM/F12 medium and StemFit® For Differentiation medium present in about a 3.9 to 0.9 ratio to about a 4.1-1.1 ratio, preferably about a 4:1 ratio, and further comprises one or more of L-glutamine, SCF, IL-6, and/or EPO, and optionally penicillin/streptomycin. The EPO is prepared from a stock concentration of 500 IU/mL to a final concentration of 10 IU/mL (e.g., 240 μL EPO stock solution is added to a six-well tissue culture plate). The morphology of the cells can be determined at the beginning of the culture period and/or at regular intervals (e.g., at 8 days of redifferentiation) by microscopy, e.g., inverted phase contrast microscopy as shown in FIGS. 8A-8MM.

In the 2D protocol, after about 7.8 days to about 8.2 days, preferably about 8 days, of redifferentiation, non-adherent iHSCs in the medium can be collected, centrifuged, resuspended in the fourth cell culture medium, and then added back to the culture.

After about 10 days of redifferentiation in the 2D protocol and the 3D/2D protocol, the appearance and morphology of the iHSCs and/or EBs can be monitored by microscopy, e.g., inverted phase contrast microscopy as shown in FIGS. 8A-8MM.

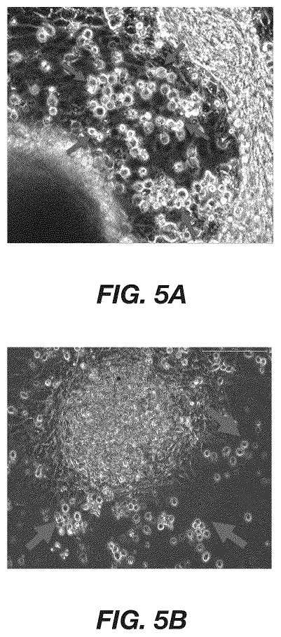

The redifferentiated iHSCs from the 2D protocol can be harvested in two fractions: non-adherent cells and adherent cells (cells obtained from the 2D protocol are shown in FIG. 5A).

The non-adherent iHSCs from the 2D protocol can be harvested and added back to the culture medium. For example, the non-adherent iHSCs from the 2D protocol can be harvested by centrifugation (e.g., at 300×g for 5 minutes) and resuspended in a fifth cell culture medium, the fifth cell culture medium comprising Advanced DMEM/F12 medium and StemFit® For Differentiation medium in about a 3.9 to 0.9 ratio to about a 4.1 to 1.1 ratio, preferably about a 4:1 ratio, and further comprises L-glutamine and optionally penicillin/streptomycin, but does not have cytokines or growth factors. The iHSCs can be counted as described herein. Optionally, the non-adherent cell fraction is not cultured further. Preferably, the non-adherent cell fraction is added back to the culture. The resulting cells can be assayed for cell markers (e.g., CD34) and/or lineage markers (e.g., CD3, CD19, CD14, CD11b, CD11c, CD56, CD20, CD16, CD2, and/or CD235a).

The adherent iHSCs from the 2D protocol can be gently dispersed using a pipet or equivalent thereof, and can optionally be pooled and dispersed again before being resuspended in the fifth culture medium following the dispersion. The dispersed cells can then be passed through two cell strainers, the first cell strainer having a mesh size of 70 μm and the second cell strainer having a mesh size of 40 μm. The cells can be counted as described herein. The resulting cells can be assayed for cell markers (e.g., CD34) and/or lineage markers (e.g., CD3, CD19, CD14, CD11b, CD11c, CD56, CD20, CD16, CD2, and/or CD235a).

After the two fractions of iHSCs from the 2D protocol are harvested in the fifth culture medium, the cells can be assessed for purity, e.g., using flow cytometry or an equivalent, to analyze the presence or absence of certain markers. The purity can be defined by the presence of CD34 expression (CD34+) and the absence of expression of one or more lineage markers CD3, CD19, CD14, CD11b, CD11c, CD56, CD20, CD16, CD2, and CD235a. In an embodiment, each of lineage markers CD3, CD19, CD14, CD11b, CD11c, CD56, CD20, CD16, CD2, and CD235a are absent. In other words, the harvested cells are CD34+ and lineage-marker negative. In an exemplary embodiment, a gating scheme to determine purity of the iHSCs comprises gating on lineage maker-negative cells followed by gating on CD34+. Following the purity analysis, the obtained iHSCs can be cryopreserved. Exemplary success criteria include, for example, γδ T-iPSC to iHSC redifferentiation having a purity greater than 70% Lin-CD34+ cells and a fold expansion/input iPSC greater than 100 for 2D cultures, and iHSC to iγδ T cell differentiation having a purity of 20% to 40% CD3+TCR γδ+ cells and a fold expansion/input iCD34 greater than 5.

The redifferentiated iHSCs and/or EBs from the 3D/2D protocol can be harvested by collection by centrifugation, gently resuspended and incubated in about 1 mL of about 18 mg/mL to about 20 mg/mL collagenase type II for about 20 minutes, followed by adding TrypLE and incubating for about 20 minutes (FIG. 3). The cell suspension resulting from the incubation in collagenase type II and TrypLE can be gently dispersed by pipetting and mixing, followed by addition of a fifth cell culture medium comprising Advanced DMEM/F12 medium and StemFit® For Differentiation medium in about a 3.9 to 0.9 ratio to about a 4.1 to 1.1 ratio, preferably about a 4:1 ratio, and further comprises L-glutamine and optionally penicillin/streptomycin, but does not have cytokines or growth factors. The mixed cell suspension can then be centrifuged to produce a cell pellet and resuspended in the fifth cell culture medium to form a second cell suspension. The second cell suspension can be passed through two cell strainers, the first cell strainer having a mesh size of 70 μm and the second cell strainer having a mesh size of 40 μm. The iHSCs can be counted as described herein and can be assayed for cell markers (e.g., CD34) and/or lineage markers (e.g., CD3, CD19, CD14, CD11b, CD11c, CD56, CD20, CD16, CD2, and/or CD235a). For example, the iHSCs can be assessed for purity, e.g., using flow cytometry or an equivalent, to analyze the presence or absence of certain markers. The purity can be defined by the presence of CD34 expression (CD34+) and the absence of expression of one or more lineage markers CD3, CD19, CD14, CD11b, CD11c, CD56, CD20, CD16, CD2, and CD235a. In an embodiment, each of lineage markers CD3, CD19, CD14, CD11b, CD11c, CD56, CD20, CD16, CD2, and CD235a are absent. In other words, the harvested cells are CD34+ and lineage marker-negative. In an exemplary embodiment, a gating scheme to determine purity of the iHSCs comprises gating on lineage maker-negative cells followed by gating on CD34+. Following the purity analysis, the obtained iHSCs can be cryopreserved. Exemplary success criteria include, for example, γδ T-iPSC to iHSC redifferentiation having a purity greater than 70% Lin-CD34+ cells and a fold expansion/iPSC greater than 0.5 for 3D/2D cultures, and iHSC to iγδ T cell differentiation having a purity of 20% to 40% CD3+TCR γδ+ cells and a fold expansion/iCD34 greater than 5.

Redifferentiation of the iHSCs into iγδ T Cells

An exemplary method of redifferentiating the iHSCs obtained by the methods described herein into iγδ T cells follows.

Appropriate planar surfaces, such as tissue or cell culture plates or vessels, can be prepared by coating the surface (e.g., wells) with one or more Notch ligand proteins and/or and vascular cell adhesion molecule 1 (VCAM1). A preferred tissue culture vessel includes a 48-well tissue culture plate. Preferred Notch ligand proteins include delta like canonical Notch ligand 1 (DLL1), DLL4, VCAM1, and Jagged 2, and particularly preferred proteins include a combination of VCAM1 and DLL4. The coating can be performed by any method known in the art.

Cryopreserved iHSCs can be thawed in StemSpan™ Lymphoid Progenitor Expansion Medium (StemCell Technologies). Cells can be counted and their viability determined as discussed herein.

Approximately 1.2×10⁴ live cells can be seeded per 250 μL of StemSpan™ Lymphoid Progenitor Expansion Medium per well, and the cells can be incubated at 37° C., 5% CO₂, 18% O₂ for further culture and differentiation. Alternatively, in one aspect, the cells are incubated under hypoxic conditions.

Additional StemSpan™ Lymphoid Progenitor Expansion Medium can be added during the culture process as commonly known in the art. The cells can be analyzed during the culture process for viability and/or T cell lineage marker expression by any method known in the art. The culture medium can be changed as commonly known in the art. For example, the culture medium can be changed every three days, e.g., on Days 13, 17, and 20 of the culture process. The cells can be harvested approximately 14 days after thawing.

### Cells Produced by the Methods

In another aspect, the invention provides one or more cells obtained from any of the methods described herein. The resulting iHSCs have high purity, viability, fold expansion, and potential to redifferentiate into immune effectors as shown in FIGS. 5A-5B, 6A-6C, and 7A-7B. The iHSCs produced by any of these methods can be further redifferentiated to lymphocytes, including iγδ T cells.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method comprising: seeding human γδ T cell derived iPSCs in a tissue culture vessel wherein the iPSCs are maintained for up to about 6 days; initiating redifferentiation of the iPSCs to iHSCs by culturing in the tissue culture vessel for about 2 days to obtain iPSC-derived cell intermediates; culturing the iPSC-derived cell intermediates for about 8 days; collecting non-adherent iPSC-derived cell intermediates and adding them back to the culture; and harvesting redifferentiated iHSCs at about day 10 of culture into two different cellular fractions, a non-adherent cellular fraction and an adherent cellular fraction.

In some embodiments, the method of making the cells further comprises the step of initiating redifferentiation of the iPSCs in a composition comprising basic fibroblast growth factor (bFGF), a glycogen synthase kinase inhibitor, a bone morphogenetic protein (BMP4), and vascular endothelial growth factor (VEGF).

In some embodiments, the method of making the cells further comprises culturing the iPSC-derived cell intermediates for about 2 days in a composition comprising effective amounts of L-glutamine, an inhibitor of the Activin/BMP/TGFβ pathway), VEGF, bFGF, and, optionally, a stem cell factor (SCF) and/or antibiotic(s).

In some embodiments, the methods further comprise culturing the iPSC-derived cell intermediates for about 2 days in a composition comprising L-glutamine, SCF, VEGF, FMS-like tyrosine kinase 3 ligand (Flt3L), erythropoietin (EPO), interleukin-3 (IL-3), interleukin-6 (IL-6), and optionally antibiotic(s).

In some embodiments, the methods further comprise culturing the iPSC-derived cell intermediates for about 2 days in a composition comprising L-glutamine, SCF, IL-6, EPO, and optionally antibiotic(s).

In some embodiments, the methods further comprise culturing redifferentiated iHSCs for about 2 days in a composition comprising L-glutamine and optionally antibiotic(s), wherein the composition does not comprise cytokines or growth factors.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, wherein said method comprises:
i) obtaining at least one human γδ T cell derived iPSC, and
wherein the method further comprises one or more of the following steps:
a) seeding the iPSCs at about 1.7×10³ cells/well to about 2.5×10³ cells per well, preferably about 2×10³ cells/well of a six-well tissue culture vessel or an equivalent thereof;
b) maintaining the iPSCs under culture conditions for about 5.5 to about 6.5 days, preferably up to 6 days;
c) initiating redifferentiation of the iPSCs into iHSCs by culturing the iPSCs for about 1.8 days to about 2.2 days, preferably about 2 days in a first cell culture medium to obtain iPSC-derived cell intermediates, wherein said first cell culture medium is StemFit® Basic 04 medium supplemented with basic fibroblast growth factor (bFGF) to a final concentration of about 90 ng/mL to about 110 ng/mL bFGF, preferably about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the iPSC-derived cell intermediates for an additional about 1.8 days to about 2.2 days, preferably about 2 days in a second cell culture medium, wherein said second cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
e) culturing the iPSC-derived cell intermediates for an additional about 1.8 days to about 2.2 days, preferably about 2 days in a third cell culture medium, wherein said third cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the iPSC-derived cell intermediates for an additional about 1.8 days to about 4.2 days, preferably about 2 days to about 4 days in a fourth cell culture medium, wherein said fourth cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SCF, IL-6, EPO, and optionally penicillin/streptomycin;
g) after about 7.8 days to about 8.2 days, preferably 8 days, of redifferentiation, collecting non-adherent iPSC-derived cell intermediates from the fourth cell culture medium and adding them back to the culture in the fourth cell culture medium;
h) harvesting redifferentiated iHSCs at day 10 of culture in two different cellular fractions, a non-adherent cellular fraction and an adherent cellular fraction, wherein the redifferentiated iHSCs are harvested in a fifth cell culture medium, wherein the fifth cell culture medium comprises Advanced DMEM/F12 medium and StemFit® For Differentiation medium in about a 3.9 to 0.9 ratio to about a 4.1-1.1 ratio, preferably about a 4:1 ratio, and further comprises L-glutamine and optionally penicillin/streptomycin, wherein the fifth cell culture medium does not comprise cytokines or growth factors;
i) harvesting non-adherent iHSCs by centrifugation, wherein said harvesting optionally does not include further culturing;
j) harvesting adherent iHSCs by dispersing cells, optionally resuspending the cells obtained from dispersion, followed by passing through a first cell strainer and a second cell strainer, wherein the first cell strainer is 70 μm mesh size and the second cell strainer is 40 μm mesh size;
k) determining the purity of the iHSCs, said purity defined as CD34+ and lineage marker-negative;
l) not introducing serum in any of steps a-k;
m) not introducing any additional cells, including feeder cells, in any of steps a-1; or
n) obtaining de novo generated iHSCs.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method comprising: a) seeding the iPSCs at about 0.4×10⁶ cells/well to about 1.2×10⁶ cells/well of a six-well ultra low adhesion tissue culture vessel or an equivalent thereof; b) culturing the iPSCs for up to about 48 hours with agitation to generate embryoid bodies (EBs); c) transferring the EBs to a tissue culture treated culture vessel and initiating redifferentiation of the EBs for about 10 days, wherein the redifferentiation culture vessel comprises EBs and cells in suspension; d) obtaining the EBs and cells in suspension; and e) optionally, treating the EBs and cells in suspension to obtain a single cell suspension.

In some embodiments, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method further comprising: obtaining CD34+ and lineage marker-negative iHSCs.

In some embodiments, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method further comprising: obtaining iHSCs that are CD34+, and CD3−, and/or CD19−, and/or CD14−, and/or CD11b−, and/or CD11c−, and/or CD56−, and/or CD20−, and/or CD16−, and/or CD2−, and/or CD235a− cells.

In an aspect, the invention provides one or more cells obtained from a method of de novo generation of hematopoietic stem cells (iHSCs) derived from human γδ T cell derived induced pluripotent stem cells (iPSCs) under serum-free, feeder-free culture conditions, said method comprising:
i) obtaining one or more human γδ T cell derived iPSCs, and
wherein the method further comprises one or more of the following steps:
   a) seeding the iPSCs at about 0.4×10⁶ cells/well to about 1.2×10⁶ cells/well, preferably about 0.6×10⁶ cells/well, of a six-well ultra low adhesion tissue culture vessel or an equivalent thereof;
   b) maintaining the iPSCs in culture conditions for up to 48 hours with agitation to generate embryoid bodies (EBs);
   c) transferring the EBs from the ultra low adhesion tissue culture vessel to a tissue culture treated tissue culture vessel and initiating redifferentiation of the EBs by culturing the EBs for about 2 days in a first cell culture medium, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
   d) culturing EBs and cells in suspension for an additional about 2 days in a second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
   e) culturing the EBs and cells in suspension for an additional about 2 days in a third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
   f) culturing the EBs and cells in suspension for an additional about 2 days to about 4 days in a fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
   g) after about 10 days of redifferentiation, harvesting the EBs and cells in suspension by centrifugation, thereby forming pelleted EBs and cells in suspension;
   h) resuspending the pelleted EBs and cells in suspension in a volume of about 20 mg/mL collagenase type II and incubating the EBs and iHSCs in the collagenase type II;
   i) adding TrypLE to the EBs and the cells in suspension and incubating the EBs and the cells in suspension to obtain a single cell suspension,
   j) mixing the single cell suspension;
   k) adding a fifth cell culture medium to the cell suspension, wherein said fifth cell culture medium comprises Advanced DMEM/F12 medium and StemFit® For Differentiation medium in a ratio of about a 4:1 ratio, and further comprises L-glutamine and optionally penicillin/streptomycin, wherein the fifth cell culture medium does not comprise cytokines or growth factors,
   l) centrifuging the cell suspension to form a cell pellet and resuspending the cell pellet in the fifth cell culture medium to form a second cell suspension;
   m) passing the second cell suspension through a first cell strainer and a second cell strainer to obtain iHSCs, wherein the first cell strainer is 70 μm mesh size and the second cell strainer is 40 μm mesh size;
   n) determining the purity of the iHSCs, said purity defined as CD34+ and lineage marker-negative;
   o) not introducing serum in any of steps a-n;
   p) not introducing any additional cells, including feeder cells or stromal cells, in any of steps a-1; or
   q) obtaining de novo generated iHSCs.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method comprising: a) a means for seeding human γδ T cell derived iPSCs in a tissue culture vessel wherein the iPSCs are maintained for up to about 6 days; b) a means for initiating redifferentiation of the iPSCs to iHSCs by culturing in the tissue culture vessel for about 2 days to obtain iPSC-derived cell intermediates; c) a means for culturing the iPSC-derived cell intermediates for about 8 days; d) a means for collecting non-adherent iPSC-derived cell intermediates and adding them back to the culture; and e) a means for harvesting redifferentiated iHSCs at about day 10 of culture into two different cellular fractions, a non-adherent cellular fraction and an adherent cellular fraction.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated culture vessel and initiating redifferentiation of the EBs for about 10 days, wherein the tissue culture treated culture vessel comprises EBs and cells in suspension; d) a means for obtaining the EBs and cells in suspension; and e) optionally, a means for treating the EBs and cells in suspension to obtain a single cell suspension.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated culture vessel and initiating redifferentiation of the EBs for about 10 days, wherein the redifferentiation culture vessel comprises EBs and cells in suspension; d) a means for obtaining the EBs and cells in suspension; e) optionally, a means for treating the EBs and cells in suspension to obtain a single cell suspension; and f) a means for providing CD34+ and lineage marker-negative iHSCs.

In an aspect, the invention provides one or more cells obtained from a method of de novo generation of hematopoietic stem cells (iHSCs) derived from human γδ T cell derived induced pluripotent stem cells (iPSCs) under serum-free and feeder-free culture conditions, said method comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated culture vessel and initiating redifferentiation of the EBs for about 10 days, wherein the tissue culture treated culture vessel comprises EBs and cells in suspension; d) a means for obtaining the EBs and cells in suspension; e) optionally, a means for treating the EBs and cells in suspension to obtain a single cell suspension; and f) a means for obtaining CD34+, and CD3−, and/or CD19−, and/or CD14−, and/or CD11b−, and/or CD11c−, and/or CD56−, and/or CD20−, and/or CD16−, and/or CD2−, and/or CD235a− iHSCs.

In some embodiments, the one or more cells have high purity, viability, fold expansion, and potential to redifferentiate into immune effectors, including iPSC-derived γδ (iγδ) T cells.

In some embodiments, the step of determining purity of the iHSCs comprises assaying for one or more, optionally all, of the lineage markers CD3, CD19, CD14, CD11b, CD11c, CD56, CD20, CD16, CD2, and CD235a via flow cytometry or an equivalent thereof.

In some embodiments, the lineage markers comprise one or more of CD3, CD19, CD14, CD11b, CD11c, CD56, CD20, CD16, CD2, and CD235a.

In some embodiments, the step of mixing the single cell suspension comprises gently dispersing the cells using a pipet or equivalent thereof.

In another aspect, the invention provides a composition comprising a cell produced by any of the methods described herein.

In another aspect, the invention provides a use of cells obtained by any of the methods described herein, in preparation of cells for treating a pathology, disease(s), in preparation of lymphocytes, in a bioreactor, in tissue engineering or in vitro drug screening for diseases.

### Systems for Carrying Out the Methods

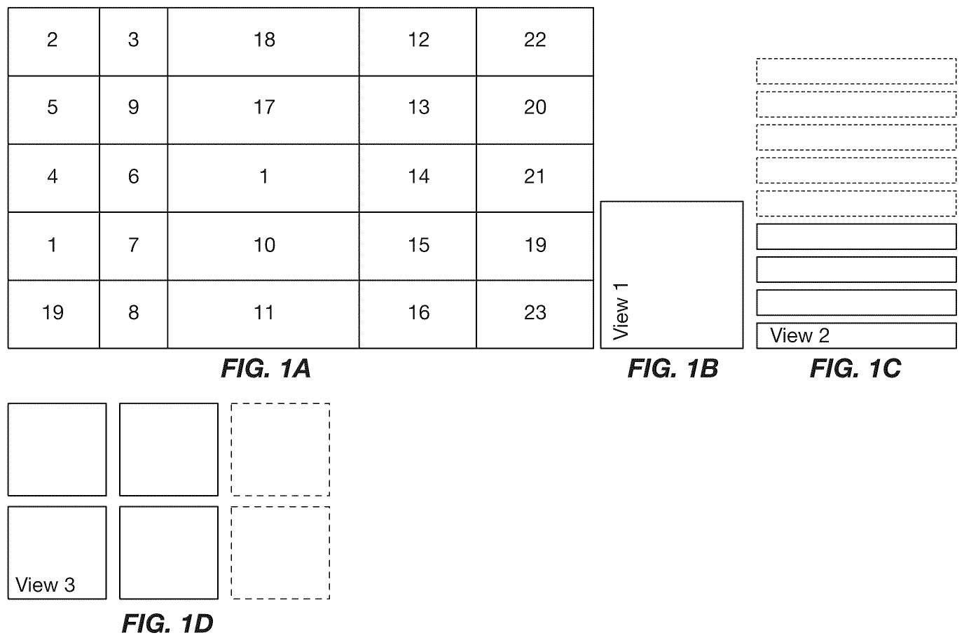

In another aspect, the invention provides a system, wherein any of the methods described herein are performed by hand or with automated robotic assistance or a combination thereof as shown in FIGS. 1A-1D.

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said method comprising: seeding human γδ T cell derived iPSCs in a tissue culture vessel wherein the iPSCs are maintained for up to about 6 days; initiating redifferentiation of the iPSCs to iHSCs by culturing in the tissue culture vessel for about 2 days to obtain iPSC-derived cell intermediates; culturing the iPSC-derived cell intermediates for about 8 days; collecting non-adherent iPSC-derived cell intermediates and adding them back to the culture; and harvesting redifferentiated iHSCs at about day 10 of culture into two different cellular fractions, a non-adherent cellular fraction and an adherent cellular fraction. The system of the present invention is mobile or stationary, or a combination thereof.

In some embodiments, the method further comprises the step of initiating redifferentiation of the iPSCs in a composition comprising basic fibroblast growth factor (bFGF), a glycogen synthase kinase inhibitor, a bone morphogenetic protein (BMP4), and vascular endothelial growth factor (VEGF).

In some embodiments, the method further comprises culturing the iPSC-derived cell intermediates for about 2 days in a composition comprising effective amounts of L-glutamine, an inhibitor of the Activin/BMP/TGFβ pathway), VEGF, bFGF, and, optionally, a stem cell factor (SCF) and/or antibiotic(s).

In some embodiments, the methods further comprise culturing the iPSC-derived cell intermediates for about 2 days in a composition comprising L-glutamine, SCF, VEGF, FMS-like tyrosine kinase 3 ligand (Flt3L), erythropoietin (EPO), interleukin-3 (IL-3), interleukin-6 (IL-6), and optionally antibiotic(s).

In some embodiments, the methods further comprise culturing the iPSC-derived cell intermediates for about 2 days in a composition comprising L-glutamine, SCF, IL-6, EPO, and optionally antibiotic(s).

In some embodiments, the methods further comprise culturing redifferentiated iHSCs for about 2 days in a composition comprising L-glutamine and optionally antibiotic(s), wherein the composition does not comprise cytokines or growth factors.

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said system comprising one or more components capable of performing a method comprising:
obtaining at least one human γδ T cell derived iPSC, and
wherein the method further comprises one or more of the following steps:
a) seeding the iPSCs at about 1.7×10³ cells/well to about 2.5×10³ cells per well, preferably about 2×10³ cells/well of a six-well tissue culture vessel or an equivalent thereof;
b) maintaining the iPSCs as colonies under culture conditions for about 5.5 to about 6.5 days, preferably up to 6 days;
c) initiating redifferentiaion of the iPSCs into iHSCs by culturing the iPSCs for about 1.8 days to about 2.2 days, preferably about 2 days in a first cell culture medium to obtain iPSC-derived cell intermediates, wherein said first cell culture medium comprises StemFit® Basic 04 medium supplemented with basic fibroblast growth factor (bFGF) to a final concentration of about 90 ng/mL to about 110 ng/mL bFGF, preferably about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the iPSC-derived cell intermediates for an additional about 1.8 days to about 2.2 days, preferably about 2 days in a second cell culture medium, wherein said second cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
e) culturing the iPSC-derived cell intermediates for an additional about 1.8 days to about 2.2 days, preferably about 2 days in a third cell culture medium, wherein said third cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the iPSC-derived cell intermediates for an additional about 1.8 days to about 4.2 days, preferably about 2 days to about 4 days in a fourth cell culture medium, wherein said fourth cell culture medium comprises Advanced DMEM/F12 medium and StemFit® For Differentiation medium present in about a 3.9 to 0.9 ratio to about a 4.1-1.1 ratio, preferably about a 4:1 ratio, and further comprises L-glutamine, SCF, IL-6, EPO, and optionally penicillin/streptomycin;
g) after about 7.8 days to about 8.2 days, preferably 8 days, of redifferentiation, collecting non-adherent iPSC-derived cell intermediates from the fourth cell culture medium and adding them back to the culture in the fourth cell culture medium;
h) harvesting redifferentiated iHSCs at day 10 of culture in two different cellular fractions, a non-adherent cellular fraction and an adherent cellular fraction, wherein the redifferentiated iHSCs are harvested in a fifth cell culture medium, wherein the fifth cell culture medium comprises Advanced DMEM/F12 medium and StemFit® For Differentiation medium in about a 3.9 to 0.9 ratio to about a 4.1 to 1.1 ratio, preferably abut a 4:1 ratio, and further comprises L-glutamine and optionally penicillin/streptomycin, wherein the fifth cell culture medium does not comprise cytokines or growth factors;
i) harvesting non-adherent iHSCs by centrifugation, wherein said harvesting optionally does not include further culturing;
j) harvesting adherent iHSCs by dispersing cells, optionally resuspending the cells obtained from dispersion, followed by passing through a first cell strainer and a second cell strainer, wherein the first cell strainer is 70 μm mesh size and the second cell strainer is 40 μm mesh size;
k) determining the purity of the iHSCs, said purity defined as CD34+ and lineage marker-negative;
l) not introducing serum in any of steps a-k;
m) not introducing any additional cells, including feeder cells, in any of steps a-1; or
n) obtaining de novo generated (iHSCs).

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said method comprising: a) seeding the iPSCs at about 0.4×10⁶ cells/well to about 1.2×10⁶ cells/well of a six-well ultra low adhesion tissue culture vessel or an equivalent thereof; b) culturing the iPSCs for up to about 48 hours with agitation to generate embryoid bodies (EBs); c) transferring the EBs to a tissue culture treated culture vessel and initiating redifferentiation of the EBs for about 10 days, wherein the redifferentiation culture vessel comprises EBs and cells in suspension; d) obtaining the EBs and cells in suspension; and e) optionally, treating the EBs and cells in suspension to obtain a single cell suspension.

In some embodiments, the invention provides a system for performing a method of de novo generation of hematopoietic stem cells (iHSCs) derived from human γδ T cell derived induced pluripotent stem cells (iPSCs) under serum-free and feeder-free culture conditions, said method further comprising obtaining CD34+ and lineage marker-negative iHSCs.

In some embodiments, the invention provides a system for performing a method of de novo generation of hematopoietic stem cells (iHSCs) derived from human γδ T cell derived induced pluripotent stem cells (iPSCs) under serum-free and feeder-free culture conditions, said method further comprising: obtaining iHSCs that are CD34+, and CD3−, and/or CD19−, and/or CD14−, and/or CD11b−, and/or CD11c−, and/or CD56−, and/or CD20−, and/or CD16−, and/or CD2−, and/or CD235a− iHSCs.

In an aspect, the invention provides a system for performing a method of de novo generation of hematopoietic stem cells (iHSCs) derived from human γδ T cell derived induced pluripotent stem cells (iPSCs) under serum-free, feeder-free culture conditions, said method comprising:
i) obtaining one or more human γδ T cell derived iPSCs, and
wherein the method further comprises one or more of the following steps:
   a) seeding the iPSCs at about 0.4×10⁶ cells/well to about 1.2×10⁶ cells/well, preferably about 0.6×10⁶ cells/well, of a six-well ultra low adhesion tissue culture vessel or an equivalent thereof;
   b) maintaining the iPSCs in culture conditions for up to 48 hours to generate embryoid bodies (EBs);
   c) transferring the EBs from the ultra low adhesion tissue culture vessel to a tissue culture treated tissue culture vessel and redifferentiating the EBs by culturing the EBs for about 2 days in a first cell culture medium, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
   d) culturing EBs and cells in suspension for an additional about 2 days in a second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
   e) culturing the EBs and cells in suspension for an additional about 2 days in a third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), optionally penicillin/streptomycin;
   f) culturing the EBs and cells in suspension for an additional about 2 days to about 4 days in a fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, optionally penicillin/streptomycin;
   g) after about 10 days of redifferentiation, harvesting the EBs and cells in suspension by centrifugation, thereby forming pelleted EBs and cells in suspension;
   h) resuspending the pelleted EBs and cells in suspension in a volume of about 20 mg/mL collagenase type II and incubating the EBs and cells in suspension in the collagenase type II;
   i) adding TrypLE to the EBs and cells in suspension and incubating the EBs and cells in suspension to obtain a single cell suspension,
   j) mixing the single cell suspension;
   k) adding a fifth cell culture medium to the cell suspension, wherein said fifth cell culture medium comprises Advanced DMEM/F12 medium and StemFit® For Differentiation medium in a ratio of about a 4:1 ratio, and further comprises L-glutamine and optionally penicillin/streptomycin, wherein the fifth cell culture medium does not comprise cytokines or growth factors,
   l) centrifuging the cell suspension to form a cell pellet and resuspending the cell pellet in the fifth cell culture medium to form a second cell suspension;
   m) passing the second cell suspension through a first cell strainer and a second cell strainer to obtain iHSCs, wherein the first cell strainer is 70 μm mesh size and the second cell strainer is 40 μm mesh size;
   n) determining the purity of the iHSCs, said purity defined as CD34+ and lineage marker-negative;
   o) not introducing serum in any of steps a-n;
   p) not introducing any additional cells, including feeder cells or stromal cells, in any of steps a-o; or
   q) obtaining de novo generated iHSCs.

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said system comprising: a) a means for seeding human γδ T cell derived iPSCs in a tissue culture vessel wherein the iPSCs are maintained for up to about 6 days; b) a means for initiating redifferentiation of the iPSCs to iHSCs by culturing in the tissue culture vessel for about 2 days to obtain iPSC-derived cell intermediates; c) a means for culturing the iPSC-derived cell intermediates for about 8 days; d) a means for collecting non-adherent iPSC-derived cell intermediates and adding them back to the culture; and e) a means for harvesting redifferentiated iHSCs at about day 10 of culture into two different cellular fractions, a non-adherent cellular fraction and an adherent cellular fraction.

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said system comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated culture vessel and initiating redifferentiation of the EBs for about 10 days, wherein the tissue culture treated culture vessel comprises EBs and cells in suspension; d) a means for obtaining the EBs and cells in suspension; and e) optionally, a means for treating the EBs and cells in suspension to obtain a single cell suspension.

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said system comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated culture vessel and initiating redifferentiation of the EBs for about 10 days, wherein the tissue culture treated culture vessel comprises EBs and cells in suspension; d) a means for obtaining the EBs and cells in suspension; e) optionally, a means for treating the EBs and cells in suspension to obtain a single cell suspension; and f) a means for providing CD34+ and lineage marker-negative iHSCs.

In an aspect, the invention provides a system for performing a method of de novo generation of hematopoietic stem cells (iHSCs) derived from human γδ T cell derived induced pluripotent stem cells (iPSCs) under serum-free and feeder-free culture conditions, said system comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated culture vessel and initiating redifferentiation of the EBs for about 10 days, wherein the tissue culture treated culture vessel comprises EBs and cells in suspension; d) a means for obtaining the EBs and cells in suspension; e) optionally, a means for treating the EBs and cells in suspension to obtain a single cell suspension; and f) a means for obtaining CD34+, and CD3−, and/or CD19−, and/or CD14−, and/or CD11b−, and/or CD11c−, and/or CD56−, and/or CD20−, and/or CD16−, and/or CD2−, and/or CD235a− iHSCs.

In some embodiments, the step of determining purity of the iHSCs comprises assaying for one or more, optionally all, of the lineage markers CD3, CD19, CD14, CD11b, CD11c, CD56, CD20, CD16, CD2, and CD235a via flow cytometry or an equivalent thereof.

In some embodiments, the lineage markers comprise one or more of CD3, CD19, CD14, CD11b, CD11c, CD56, CD20, CD16, CD2, and CD235a.

In some embodiments, the step of mixing the single cell suspension comprises gently dispersing the cells using a pipet or equivalent thereof.

In some embodiments, the system is automated or semi-automated. Suitable systems are described in, e.g., Maria Kempner and Robin Felder, A Review of Cell Culture Automation, JALA: Journal of the Association for Laboratory Automation, vol. 7, issue 2, April 2002, p. 56-62; and Miho Sasamata et al., Establishment of a Robust Platform for Induced Pluripotent Stem Cell Research Using Maholo LabDroid, SLAS Technology 2021, Vol. 26(5), p. 441-453, each of which is herein incorporated by reference as if set forth in its entirety.

In some embodiments, the system comprises one or more software packages, the software package(s) operating and scheduling operation of the system.

In some embodiments, the software package(s) are customized and or customizable for desired applications and are, optionally, menu-driven.

In some embodiments, the automation comprises cell visualization, plate handling, plate coating, seeding, extraction, addition, cell feeding, incubation assays and or sampling.

In some embodiments, the system comprises one or more incubators on line.

In some embodiments, the system comprises electronic humidity controls, a HEPA filter system, a carousel, said carousel comprising programmable stepping, oscillation cycles and or a two-way communication interface or any combination thereof.

In some embodiments, medium is added or removed or supplemented without disturbing or contaminating cells.

In some embodiments, the system comprises one or more computer operated and controlled robotic arms.

### Elements of a Nonlimiting Exemplary Automated System

A nonlimiting exemplary automated system is shown in FIGS. 1A-1D. The elements shown in FIG. 1A are, in several exemplary non-limiting embodiments, as follows: 1. Actuator; 2. Cooler; 3. Freezer; 4. Track; 5. Incubator (e.g., with and without inlets and outlets); 6. Transferor; 7. to 9. Contactor(s); 10. Vessel Retainer; 11. Lifter; 12. Tubular Retainer; 13. Tubular Retainer; 14. Tubular Retainer; 15. Tubular Retainer; 16. Warmer; 17. Transferor; 18. Mixer; 19. Storer; 20. Visualizer; 21. Fluid Manipulator; 22. Separator; and 23. Phenotyper. One or more of these elements are linked, e.g., operably linked, to one another as known in the art. For example, one or more of the elements can be linked electrically and/or mechanically, via gas lines, via waste lines, etc.

One of ordinary skill in the art understands that one or more of these elements are combinable in different arrangements depending on the desired method of generating iHSCs according to the general knowledge in the art. For example, the elements can be stacked vertically or can be side by side, or can be in combinations of such arrangements. One or more of these elements can be computer or software operated and/or controlled, alone or in operable combination with another element. One or more of these elements can be operated manually. Exemplary automated systems incorporating one or more of the above elements are described in, e.g., Maria Kempner and Robin Felder, A Review of Cell Culture Automation, JALA: Journal of the Association for Laboratory Automation, vol. 7, issue 2, April 2002, p. 56-62; and Miho Sasamata et al., Establishment of a Robust Platform for Induced Pluripotent Stem Cell Research Using Maholo LabDroid, SLAS Technology 2021, Vol. 26(5), p. 441-453, each of which is herein incorporated by reference as if set forth in its entirety.

The methods of the invention may be carried out in an appropriate system that comprises one or more of the elements described herein to obtain the cells described herein. One or more of the element(s) of the invention taught herein are configured in operable combination with another element(s). The system can be of any size (e.g., small scale or large scale) or type (e.g., open, closed, batch, fed-batch, perfusion, chemostat, continuous) as long as it is useful for culturing cells, e.g., mammalian cells or human cells. The culture unit can provide suitable culture conditions as described herein, e.g., agitation, oxygen levels, and temperature, and can perform suitable actions as described herein, e.g., agitation, culture medium changes, microscopy, cell sorting, and/or flow cytometry.

Exemplary elements or components for use with the present invention include the following non-limiting sources and exemplary elements RO1 from Standard Bots (standardbots.com/ro1), UFACTORY and UFACTORY 850 (ufactory.cc/ufactory-850/), and Interbotix X-Series arms (trossenrobotics.com/robotic-arms.aspx) including various arm types such as Articulated arm, Six-axis arm, Collaborative robot arm, SCARA arm, Cartesian arm, Cylindrical arm, Spherical/Polar arm, Parallel/Delta arm, Anthropomorphic arm and Dual-arm varieties, and see also U.S. Pat. Nos. 4,806,066, 8,374,722, US20100158656, U.S. Pat. No. 6,826,977, US20120253513 and the like; cooler elements having various capacities and functionality available from, inter alia, VWR, Global Industrial, American Biotech, Galaxy, Accucold, (k2sci.com/products/2-cu-ft-combination-glass-door-refrigerator-10-cu-ft-solid-door-freezer); incubators, such as Powers Scientific, Micro Q, VWR, Thermo Scientific, Friocell, Benchmark, Jeio Tech, Stellar Scientific, Being Bit (see, for example, labincubators.net/); separators, including Beckman Coulter (beckman.com/landing/ppc/cent/benchtop/microcentrifuges), Thermo Fisher (thermofisher.com/us/en/home/life-science/lab-equipment/lab-centrifuges/benchtop-centrifuges/models.html), VWR (us.vwr.com/store/category/centrifuges) and the like; visualizers, including Leica (leica-microsystems.com/c/am/lsr-c/personal-confocal-promo), KEYENCE (keyence.com/landing/microscope/lp_all-in-one-microscope_01085523.jsp), ZEISS (.zeiss.com/metrology/products/systems/industrial-microscopy.html), etc.; and phenotypers including, for example, Nanocellect (nanocellect.com/products/wolf-g2-cell-sorter), Namocell (namocell.com), Beckman (beckman.com/landing/ppc/flow/cell-sorters), Sino Biologics (sinobiological.com/category/fcm-facs-facs), and the like, respectively. Each reference is herein incorporated in its entirety. A person having ordinary skill in the art would understand the exemplary embodiments and their usefulness with the present invention, and processes for the evaluation and inclusion of others.

### EXAMPLES

The following examples are provided to further describe some of the embodiments disclosed herein. The examples are intended to illustrate, not to limit, the disclosed embodiments.

### Example 0: Serum-Free, Feeder-Free, Two-Dimensional Redifferentiation of γδ T Cell-Derived iPSCs into iHSCs and then into iγδ T Cells

The protocol for the first redifferentiation step (iPSC to iHSC) was optimized using two γδ T cell-derived iPSC (γδ T-iPSC) clones, clones 1 and 2. Clone 2 is a subclone of clone 1. Clone 3 was used to validate the optimized protocol. As shown herein, given that iPSC clones are heterogeneous in their T cell redifferentiation potential, it is important to validate the optimized γδ T-iPSC to iHSC redifferentiation protocol using multiple iPSC clones. To this end, the optimized protocol has been validated using other fully characterized clones. The data for Clone 3 are shown in FIG. 6A-6C.

### Materials

TABLE 1
Reagents for iPSC culture and maintenance.

| Reagent | Product No. |
|---|---|
| γδ T-iPSC clones | Academic Institution, Contract |
| | Research Organization |
| iMatrix 511 | Amsbio, cat.# AMS.892 012 |
| StemFit ® Basic 04 Complete | Amsbio, cat.# SFB-504-CT |
| Type | |
| rh bFGF | Amsbio, cat.# AMS-FGF-100 |
| Y-27632 - ROCK Inhibitor, 1 mg | StemCell Technologies, cat# 72302 |
| TryPLE ™ Select 1X | ThermoFisher, cat.# 12563011 |
| 1X DPBS | ThermoFisher, cat.# 14190144 |
| CryoStor CS10 | StemCell Technologies, cat.# 07930 |

TABLE 2
Reagents needed for hematopoietic redifferentiation
(listed by day of redifferentiation)

| | |
|---|---|
| Reagent | Product No. |

Day 0 Reagents

| | |
|---|---|
| StemFit Basic 04 Complete | Amsbio, cat.# SFB-504-CT |
| CHIR99021 | TOCRIS, cat.# 4423 |
| rh BMP4 | R&D, cat.# 314-BP |
| rh VEGF | R&D, cat.# 293-VE |

Day 2 Reagents

| | |
|---|---|
| Advanced DMEM/F12 | Gibco, cat.# 12634-10 |
| StemFit ® For Differentiation | Ajinomoto, cat.# 20170228A |
| L-Glutamine | Gibco, cat.# 25030-081 |
| Penicillin-Streptomycin (P/S) | Gibco, cat.# 15140-122 |
| SB431542 | Fujifilm Wako pure chemical |
| | corporation, cat.# 033-24631 |
| rh VEGF | R&D, cat.# 293-VE |
| rh bFGF | Amsbio, cat.# AMS-FGF-100 |
| rh SCF | R&D, cat.# 255-SC |

Day 4 Reagents

| | |
|---|---|
| Advanced DMEM/F12 | Gibco, cat.# 12634-10 |
| StemFit ® For Differentiation | Ajinomoto, cat.# 20170228A |
| L-Glutamine | Gibco, cat.# 25030-081 |
| P/S | Gibco, cat.# 15140-122 |
| rh SCF | R&D, cat.# 255-SC |
| rh VEGF | R&D, cat.# 293-VE |
| rh IL-3 | PeproTech, cat.# AF-200-03 |
| rh IL-6 | R&D, cat.# 206-IL-050 |
| rh Flt3L | R&D, cat.# 308-FK-025 |
| rh EPO | R&D, cat.# 287-TC-500 |

Days 6-10 Reagents

| | |
|---|---|
| Advanced DMEM/F12 | Gibco, cat.# 12634-10 |
| StemFit ® For Differentiation | Ajinomoto, cat.# 20170228A |
| L-Glutamine | Gibco, cat.# 25030-081 |
| P/S | Gibco, cat.# 15140-122 |
| rh SCF | R&D, cat.# 255-SC |
| rh IL-6 | R&D, cat.# 206-IL-050 |
| rh EPO | R&D, cat.# 287-TC-500 |

TABLE 3
Reagents for iγδ T cell redifferentiation.

| Reagent | Product No. |
|---|---|
| StemSpan ™ SFEM II | StemCell Technologies, cat.# |
| | 09655 |
| Lymphoid Progenitor Expansion | StemCell Technologies, cat.# |
| Supplement (10X) | 09915 |
| rh VCAM-1-Fc | R&D, cat.# 862-VC-100 |
| rh DLL4-Fc | R&D, cat.# 10185-D4-050 |

TABLE 4
Supplies and consumables needed for cell culture.

| Item | Product No. |
|---|---|
| Corning ™ Costar ™ 6-well Clear TC- | Corning, cat.# 3516 |
| treated Multiple Well Plates, Individually | |
| Wrapped, Sterile | |
| Corning ™ Costar ™ 48-well Clear TC- | Corning, cat.# 3548 |
| treated Multiple Well Plates, Individually | |
| Wrapped, Sterile | |
| Fisherbrand ™ Sterile Polystyrene | ThermoFisher, |
| Disposable Serological Pipets with | cat.# 13-676-10H, |
| Magnifier Stripe (various sizes) | cat.# 13-678-11E, |
| | cat.# 13-678-11, |
| | cat.# 13-678-11F |
| Pipette Tips RT LTS 20 μL F 960A/10 | Rainin, cat.# 30389225 |
| (P20) | |
| Pipette Tips RT LTS 200 μL F 960A/10 | Rainin, cat.# 30389239 |
| (P200) | |
| Pipette Tips RT LTS 1000 μL F 768A/8 | Rainin, cat.# 30389212 |
| (P1000) | |
| 50 mL conical tubes | Corning, cat.# 352098 |
| Falcon ® 15 mL Polystyrene Centrifuge | Corning, cat.# 352095 |
| Tube, Conical Bottom, with Dome Seal | |
| Screw Cap, Sterile | |
| 1.8 mL cryovials | Thermofisher, cat.# 375418 |
| 1.5 mL Eppendorf tubes | Greiner-bio-one cat.# 616261 |
| Cell lifters | Corning, cat.# 3008 |
| Corning CoolCell ™ Freezing container | Corning, cat.# 432006 |
| Via-1-Cassette | Chemometec, cat.# 941-0012 |

TABLE 5
Antibodies for Flow Panels.

| | | | |
|---|---|---|---|
| Fluorochrome | Marker | Vendor | Cat# |

Pluripotency

| | | | |
|---|---|---|---|
| BV711 | SSEA-1 | BioLegend | 323050 |
| PE-Cy7 | SSEA-3 | BioLegend | 330326 |
| PerCP/Cy5.5 | CD34+ | BioLegend | 343522 |
| PE | SSEA-4 | BioLegend | 330406 |
| AF647 | OCT 3/4 | BioLegend | 653710 |
| BV421 | SOX2 | BioLegend | 656114 |

iHSC Panel

| | | | |
|---|---|---|---|
| PE | CD34 | BioLegend | 343506 |
| APC | CD43 | BD | 560198 |
| BUV395 | CD45 | BD | 563792 |
| BV421 | CD38 | BioLegend | 303526 |
| APC-Cy7 | Live/dead | ThermoFisher | L10119 |
| | Near IR | | |
| FITC | CD3 | BioLegend | 300306 |
| FITC | CD235a | BioLegend | 349104 |
| FITC | CD11c | BioLegend | 337214 |
| FITC | CD11b | BioLegend | 301330 |
| FITC | CD14 | BioLegend | 325604 |
| FITC | CD56 | BioLegend | 318304 |
| FITC | CD19 | BioLegend | 302206 |
| FITC | CD20 | BioLegend | 302304 |
| FITC | CD16 | BioLegend | 302006 |
| FITC | CD2 | BioLegend | 300206 |

iγδ T cell redifferentiation

| | | | |
|---|---|---|---|
| BUV395 | CD3 | BD | 740283 |
| BUV496 | CD25 | BD | 741144 |
| BUV563 | CD45RA | BD | 612926 |
| BUV615 | CD2 | BD | 751450 |
| BUV661 | CD277 (BTN3) | BD | 750227 |
| BUV737 | CD56 | BD | 612766 |
| BUV805 | CD11a | BD | 748572 |
| BV421 | CD197 (CCR7) | BioLegend | 353208 |
| BV480 | CD95 | BD | 746675 |
| BV605 | CD5 | BioLegend | 364020 |
| sBV650 | CD16 | BioLegend | 302042 |
| BV711 | HLA-ABC | BD | 565333 |
| BV786 | CD122 | BD | 743118 |
| FITC | Vγ9 TCR | BioLegend | 331306 |
| PerCP-Cy5.5, | CD215 (IL-15Rα) | Invitrogen | 46-7149-82 |
| BB700 | | | |
| PE | BTN2A1 | LSBio | LS-C649249- |
| | | | 100 |
| PE-Texas Red | NKG2D | BD | 562498 |
| PE-Cy5 | CD1a | BioLegend | 300108 |
| PE-Cy7 | CD27 | BioLegend | 356412 |
| APC, Alexa 647 | V82 TCR | BioLegend | 331418 |
| Alexa 700 | CD127 | BD | 565185 |
| APC-Cy7 | Live/dead Near IR | ThermoFisher | L10119 |

### Exemplary Procedure

1.1 Supplemented StemFit Basic 04 Complete Type Medium was prepared as follows:
   a. The StemFit Basic 04 Complete Type Medium was provided frozen and was stored at or below −20° C. until use. Sterile techniques were used to prepare Supplemented StemFit Basic 04 Complete Type Medium.
   b. Frozen StemFit Basic 04 Complete Type Medium was thawed with occasional mixing at room temperature (15-25° C.) or in a refrigerator (2-8° C.).
   c. Note: Medium was not thawed at 37° C., as it accelerates the degradation of the medium ingredients.
   d. StemFit Basic 04 Complete Type Medium was supplemented with an additional 20 ng/mL of bFGF to achieve a final concentration of 100 ng/mL bFGF in the supplemented medium.
   e. Supplemented medium was stored for up to two (2) weeks at 4° C.
   f. Medium was brought to room temperature (15-25° C.) for at least one (1) hour prior to use.
1.2 10 mM Y-27632 ROCK inhibitor stock solution was prepared as follows:
   a. Y-27632 ROCK inhibitor was received as a crystalline solid and was stored at −20° C. for up to 12 months from date of receipt.
   b. A 10 mM stock solution was prepared in 1×DPBS as follows:
      i. 312 μL of 1×DPBS was added to 1 mg Y-27632 and solution was resuspended completely.
      ii. 25 μL of stock solution was aliquoted into Eppendorf tubes labeled as Y-27632 stock.
      iii. Aliquots were stable at −20° C. for up to 6 months.
      iv. Working volumes were aliquoted to avoid repeated freeze-thaw cycles.
1.3 One 6-well TC-treated plate coated with iMatrix 511 was prepared as follows:
   a. 12 mL of 1×DPBS was pipetted into a 15 mL conical tube.
   b. 30 μL of iMatrix 511 was added to 1×DPBS and immediately mixed well (coating concentration: 0.25 μg/cm²).
   c. 2 mL of iMatrix solution was added to each well in the 6-well plate.
   d. Plates were incubated at 37° C., 5% CO₂, for at least 60 minutes (plates could be left overnight at 37° C.).
   e. Note: After coating step, plates could be stored at 4° C. for one week. Prior to use, plate was incubated at 37° C. for one hour.
1.4 Supplemented StemFit Basic 04 Complete Type Medium (from Step 1.1) with 10 μM Y-27632 ROCK inhibitor was prepared as follows:
   a. An aliquot of 10 mM Y-27632 ROCK inhibitor stock solution was thawed prior to use.
   b. 25 mL of Supplemented StemFit Basic 04 Complete Type Medium (from Step 1.1) was aliquoted into a 50 mL conical tube.
   c. 25 μL of 10 mM Y-27632 ROCK inhibitor was added to Supplemented StemFit Basic 04 Complete Type Medium to achieve a final concentration of medium with 10 μM Y-27632 ROCK inhibitor.
1.5 γδ T-iPSC vial was thawed as follows:
   a. A vial of frozen γδ T-iPSCs was thawed in a 37° C. water bath for 1 minute or until small pieces of frozen floating cells were visible.
   b. The thawed γδ T-iPSCs were transferred from the vial slowly in a drop-wise manner into a 15 mL conical tube containing 9 mL of room temperature Supplemented StemFit 04 Basic Complete Type Medium with 10 μm Y-27632 ROCK inhibitor (from Step 1.4).
   c. Note: Total volume was 10 mL.
1.6 Cell count.
   a. The NucleoCounter NC-200 instrument was used for cell counting purposes.
   b. 150 μL aliquot was removed from the cell suspension (Step 1.5.b) to a 1.5 mL Eppendorf tube.
   c. Cells were collected from Eppendorf tube into a Vial-Cassette.
   d. Vial-Cassette was inserted into the NC-200 cell counter and cells were counted.
   e. Note: Only total number of live cells were used for cell seeding purposes
1.7 Washing and plating thawed γδ T-iPSCs was performed as follows:
   a. 15 mL conical tube containing thawed γδ T-iPSCs (Step 1.5 b) was centrifuged at 300×g for 3 minutes at room temperature.
   b. Supernatant was aspirated without disturbing the cell pellet by leaving behind 0.5 mL of medium.
   c. Conical tube was gently tapped to loosen cell pellet.
   d. 1 mL of Supplemented StemFit Basic 04 Complete Type Medium with 10 μM Y-27632 ROCK inhibitor (Step 1.4) was added with P1000 to resuspend cells.
   e. Additional Supplemented StemFit Basic 04 Basic Complete Type Medium with 10 μM Y-27632 ROCK inhibitor (Step 1.4) was added to achieve a final cell concentration of 1×10⁴ cells/mL.
   f. iMatrix 511 solution was removed from the 6-well plate by aspiration.
      i. Note: leaving plate to dry out for extended period (e.g., more than 10 minutes) was avoided. Wells were not allowed to dry.
   g. Cells were thoroughly mixed and then 2 mL/well were plated.
   h. Cells were distributed evenly on the bottom of the well by manually rocking the plate gently back and forth and side to side.
   i. Plate was incubated overnight at 37° C. in 5% CO₂.
1.8 Day 1: Medium change was performed as follows:
   a. Supplemented StemFit Basic 04 Complete Type Medium (from Step 1.1) was brought from 4° C. to room temperature.
   b. Medium was gently aspirated from 6-well plate.
   c. 2 mL/well of fresh Supplemented StemFit Basic 04 Complete Type Medium (without ROCK inhibitor)(Step 1.1) was added to 6-well plate.
   d. Imaging of the plate was performed with EVOS M7000 and images were recorded at 4× and 10× magnification.
   e. Three images of different regions on the plate were taken to record the morphology and landscape of the culture. There were single cells and two-celled colonies present. Cells had a spiky morphology due to culturing in the presence of the ROCK inhibitor.
      i. Note: as cells divide, borders became more defined.
      ii. An example view at 10× magnification is shown in FIG. 8A.
1.9 Cells continued to be cultured until they reached typical morphology, exhibiting all iPSC characteristics including circular colonies with densely packed cells, high nucleus to cytoplasmic ratio, well-defined edges, and distinct borders. iPSC colonies were at ˜70-80% confluency at this stage.
   a. Note: It could take 7 days+/−1 day for the colonies to exhibit this morphology.
   b. iPSC colonies had defined borders and compact cells.
      i. An example view at 10× magnification is shown in FIG. 8B.
1.10 Cells were passaged and pluripotency was confirmed by flow cytometry before initiating redifferentiation:
   a. As many iMatrix 511-coated 6-well plates were prepared as required to passage iPSCs.
   b. Passaging iPSCs was performed as follows:
      i. Medium was aspirated from cells plated in Step 1.7.
      ii. Wells were rinsed with 1 mL 1×DPBS.
      iii. 1×DPBS was aspirated from wells.
      iv. 1 mL TrypLE was added and then incubated at 37° C. for 6-8 minutes.
      v. Wells were examined under a microscope. Cells appeared bright with clear cell borders as shown in FIG. 8C.
      vi. TrypLE was gently removed.
      vii. 1 mL of Supplemented StemFit Basic 04 Complete Type Medium with 10 μM Y-27632 ROCK inhibitor (from Step 1.4) was added with P1000 and then pipetted gently to remove cells from plate. Forming bubbles was avoided.
   c. A microscope was used to ensure removal of 95% of the cells from wells.
      i. If residual cells were still adherent to the plate, then 1 mL of StemFit Basic 04 Complete Type Medium with 10 μM Y-27632 ROCK inhibitor (from Step 1.4) was added. A cell lifter was then used to scrape the well, and a P1000 was used to pipette three (3) times to ensure uniformed, single cell suspension.
   d. Cells were collected into a 15 mL conical tube.
   e. Cell count was performed as in Step 1.6.
   f. 1×10⁶ cells were removed and used for pluripotency flow analysis.
   g. Pluripotency flow panel is shown in Table 5.
   h. Acceptance criteria were met before proceeding to redifferentiation steps. Acceptance criteria for pluripotency are:
      >85% Oct4+, Sox2+, SSEA3+, SSEA4+
      <1% SSEA1+
      <5% CD34+
   i. Day −6: Redifferentiation of γδ T cell-derived iPSCs into iγδ T cells: 2×10³ cells/well were plated with Supplemented StemFit Basic 04 Complete Type Medium with 10 μM Y-27632 ROCK inhibitor (Step 1.4).
      i. Note: iPSCs were passaged for at least 5 times prior to initiating redifferentiation. Maintenance of iPSCs was continued from Steps 1.10.a-h.
      ii. Depending on final cell count (Step 1.10.e), 1.2×10⁴ cells in 12 mL Supplemented StemFit Basic 04 Complete Medium with 10 μM Y-27632 ROCK inhibitor (Step 1.4) were prepared.
      iii. If there were any cells remaining after plating, they were cryopreserved in Cryostor CS10.
         a. Cells were cryopreserved in CS-10 freezing medium as follows:
         i. Cells were centrifuged at 300×g for 5 minutes at room temperature.
         ii. Supernatant was carefully removed.
         iii. Cell pellet was suspended in CS-10 medium at 1-2×10⁶ cells/mL and aliquoted at 1 mL/cryovial.
         iv. Cryovials were placed into a CoolCell freezing container and then placed into a −80° C. freezer overnight.
         v. Within 3 days, cells were transferred to Liquid N₂ tank for storage.
   j. Day −5: existing medium was replaced with fresh Supplemented StemFit Basic 04 Complete Type Medium (Step 1.1).
   k. Cells were incubated and cultured at 37° C., 5% CO₂, and medium was changed every alternative day until Day 0.
1.11 Day 0: Onset of hematopoietic redifferentiation—Mesoderm induction phase
   a. StemFit Basic 04 Complete Type Medium (Step 1.1) was pre-warmed at room temperature, and STEP 1 medium for Days 0-2 was prepared:

| | Stock | | Final | | Volume for | |
|---|---|---|---|---|---|---|
| Reagent | Concentration | | Concentration | | 6-well plate | |
| StemFit Basic 04 | | | | | 12 | mL |
| Complete Type | | | | | | |
| Medium (Step 1.1) | | | | | | |
| CHIR99021 | 20 | mM | 4 | μM | 2.4 | μL |
| rh BMP4 | 100 | μg/mL | 80 | ng/ml | 9.6 | μL |
| rh VEGF | 100 | μg/mL | 80 | ng/ml | 9.6 | μL |

b. The plate(s) were taken from the incubator and placed on an inverted phase contrast microscope to observe cell growth and morphology.
c. Images were recorded at Day 0. A 4× magnification of exemplary cells is shown in FIG. 8D, and a 10× magnification of exemplary cells is shown in FIG. 8E.
d. Medium was carefully aspirated, and 2 mL/well of STEP 1 medium (Step 1.11.a) were added to all wells.
e. The cells were cultured at 37° C., 5% CO₂.
1.12 Day 2: Onset of hematopoietic redifferentiation—Mesoderm to definitive hematopoietic lineage specification.
   a. At Day 2, Advanced DMEM/F12 medium was pre-warmed at room temperature, and STEP 2 medium for Days 2-4 was prepared:

| | Stock | | Final | | Volume for | |
|---|---|---|---|---|---|---|
| Reagent | Concentration | | Concentration | | 6-well plate | |
| Advanced DMEM/F12 | 100% | | | | 9.6 | mL |
| StemFit ® For | 100% | | 20% | | 2.4 | mL |
| Differentiation | | | | | | |
| L-Glutamine | 100X | | 1X | | 120 | μL |
| P/S | 100X | | 0.5X-1X | | 60 | μL |
| SB431542 | 5 | mM | 1.5-2.5 | μM | 4.8 | μL |
| rh VEGF | 100 | μg/mL | 80 | ng/mL | 9.6 | μL |
| bFGF | 100 | μg/mL | 50 | ng/ml | 6.0 | μL |
| SCF | 100 | μg/mL | 50 | ng/ml | 6.0 | μL |

b. The plate(s) were taken from the incubator and placed on an inverted phase contrast microscope to observe cell growth and morphology.
c. Images were recorded at Day 2 as in Step 1.11.c. A 4× magnification of exemplary cells is shown in FIG. 8F, and a 10× magnification of exemplary cells is shown in FIG. 8G.
d. Medium was carefully aspirated, and 2 mL/well of STEP 2 medium (Step 1.12.a) was added to all wells.
e. Cells were incubated and cultured at 37° C., 5% CO₂.
1.13 Day 4: Onset of hematopoietic redifferentiation—Hemangioblast induction phase
   a. At Day 4, Advanced DMEM/F12 medium was pre-warmed at room temperature, and STEP 3 medium for Days 4-6 was prepared:

| | Stock | | Final | | Volume for | |
|---|---|---|---|---|---|---|
| Reagent | Concentration | | Concentration | | 6-well plate | |
| Advanced DMEM/F12 | 100% | | | | 9.6 | mL |
| StemFit ® For | 100% | | 20% | | 2.4 | mL |
| Differentiation | | | | | | |
| L-Glutamine | 100X | | 1X | | 120 | UL |
| P/S | 100X | | 0.5X-1X | | 60 | μL |
| SCF | 100 | μg/mL | 50 | ng/mL | 6.0 | μL |
| rh VEGF | 100 | μg/mL | 20 | ng/mL | 2.4 | μL |
| rh IL-3 | 100 | μg/mL | 50 | ng/ml | 6.0 | μL |
| rh IL-6 | 100 | μg/mL | 50 | ng/ml | 6.0 | μL |
| rh Flt3L | 100 | μg/mL | 50 | ng/ml | 6.0 | μL |
| rh EPO | 500 | IU/mL | 10 | IU/mL | 240 | μL |

b. The plate(s) were taken from the incubator and placed on an inverted phase contrast microscope to observe cell growth and morphology.
c. Images were recorded at Day 4 as in Step 1.11.c. A 4× magnification of exemplary cells is shown in FIG. 8H, and a 10× magnification of exemplary cells is shown in FIG. 8I.
d. Medium was carefully aspirated, and 2 mL/well of STEP 3 medium (Step 1.13.a) were added to all wells without any wash or rinse.
e. Cells were incubated and cultured at 37° C., 5% CO₂.
1.14 Day 6: Onset of hematopoietic redifferentiation—Endothelial to hematopoietic transition phase
   a. On Days 6 and 8, Advanced DMEM/F12 medium was pre-warmed at room temperature, and fresh STEP 4 medium was prepared:

| | Stock | | Final | | Volume for | |
|---|---|---|---|---|---|---|
| Reagent | Concentration | | Concentration | | 6-well plate | |
| Advanced | 100% | | | | 9.6 | mL |
| DMEM/F12 | | | | | | |
| StemFit ® For | 100% | | 20% | | 2.4 | mL |
| Differentiation | | | | | | |
| L-Glutamine | 100X | | 1X | | 120 | μL |
| P/S | 100X | | 0.5X-1X | | 60 | μL |
| rh SCF | 100 | μg/mL | 50 | ng/mL | 6.0 | μL |
| rh IL-6 | 100 | μg/mL | 50 | ng/ml | 6.0 | μL |
| rh EPO | 500 | IU/mL | 10 | IU/mL | 240 | μL |

b. The plate(s) were taken from the incubator and placed on an inverted phase contrast microscope to observe cell growth and morphology.
c. Images were recorded at Day 6 as in Step 1.11.c. A 4× magnification of exemplary cells is shown in FIG. 8J, and a 10× magnification of exemplary cells is shown in FIG. 8K.
d. Medium was carefully aspirated, and 2 mL/well of STEP 4 medium (Step 1.14.a) were added to all wells.
e. Cells were incubated and cultured at 37° C., 5% CO₂.
f. On Day 8, 12 mL of STEP 4 medium was prepared as outlined in table above.
g. The plate(s) were taken from the incubator and placed on an inverted phase contrast microscope to observe cell growth and morphology.
h. Images were recorded at Day 8 as in Step 1.11.c. A 4× magnification of exemplary cells is shown in FIG. 8L, and a 10× magnification of exemplary cells is shown in FIG. 8M.
i. The medium was collected from all wells of the plate into a 15 mL conical tube.
   i. Medium may contain non-adherent cells.
j. 1 mL of Step 4 medium was added to each well.
k. 15 mL conical tube containing collected non-adherent cells was centrifuged at 300×g for 5 minutes at room temperature.
l. Supernatant was discarded and cell pellet was resuspended in 6 mL of fresh STEP 4 medium and 1 mL was distributed to each well to reach a final volume 2 mL per well.
m. Cells were incubated and cultured at 37° C., 5% CO₂ for another two days.
1.15 Day 10: Harvesting hematopoietic stem cells (iCD34/iHSCs) and preparing cells for flow cytometric analysis and cryopreservation was performed as follows:
   a. The plate(s) were taken from the incubator and placed on an inverted phase contrast microscope to observe cell growth and morphology.
   b. Images were recorded at Day 10 as in Step 1.11.c. A 4× magnification of exemplary cells is shown in FIG. 8N, and a 10× magnification of exemplary cells is shown in FIG. 8O.
   c. 50 mL of STEP 4 base medium (Step 1.14.a) without cytokines and growth factors were prepared.
   d. Non-adherent cell fraction was processed as follows: the plate was gently swirled and the floating cells were collected into a new 50 mL conical tube.
      i. Cells were centrifuged at 300×g for 5 minutes at room temperature.
      ii. The supernatant was discarded, and cells were resuspended in the STEP 4 base medium without added cytokines or growth factors (Step 1.15.c).
      iii. Cell count was performed using NC-200 as in Step 1.6.
   e. Adherent cell fraction was processed as follows: adherent cells were detached from the plate by gently pipetting up and down, 4-6 times, in STEP 4 base medium without added cytokines and growth factors (Step 1.15.c), using P1000.
      i. The adherent cells were collected into new 50 mL conical tube and gently resuspended with 10 mL pipette. Cells were then passed through 70 μm cell strainer followed by a 40 μm cell strainer.
   f. Note: cells from non-adherent and adherent fractions were kept separate.
   g. Cell count was performed using NC-200 as in Step 1.6.
   h. 0.5×10⁶ to 1.0×10⁶ cells were aliquoted for flow cytometric analysis using the iHSC panel.
   i. iHSC flow panel is shown in Table 5.
   j. Success criteria for iHSC redifferentiation step: purity of >70% Lineage-CD34+ cells and >100 fold expansion per input iPSC.
   k. iHSCs cells can be redifferentiated into iγδ T cells as known in the art.
   l. Alternatively, iHSCs/iCD34 cells could be cryopreserved in CS-10 freezing medium as in Step 1.10.i.iii.a.
      i. Note: Cryopreserved iHSC/iCD34 cells can be thawed and redifferentiated at a later date.

### Example 1: Serum-Free, Feeder-Free Redifferentiation of γδ T Cells from γδ T Cell-Derived Induced Pluripotent Stem Cells

### Methods

Induced Pluripotent Stem Cells (iPSCs): Healthy donor-derived peripheral blood Vγ9Vδ2 T cells were selectively expanded and then reprogrammed into iPSCs using Sendai virus-delivered Yamanaka factors following the method of Watanabe et al.³ Resulting iPSCs were expanded and banked.

iPSC Characterization: Banked γδ T-iPSCs were tested for pluripotency, genomic stability, sterility, morphology, doubling time, HLA genotype, and TCR monoclonality. F6 T-iPSCs meeting defined acceptance criteria (see Table 6) were assayed for T cell redifferentiation potential using the two-step, 2D research-scale SFFF redifferentiation protocol.

TABLE 6
Assays used to characterize iγδ T-iPSC clones

| Assays | Acceptance Criteria |
|---|---|
| Pluripotency | Pluripotency Markers (Flow): >85% expression of |
| | Oct4, Sox2, SSEA3, SSEA4, <1% SSEA1, <5% CD34 |
| | PluriTest ™: Pluripotency Score >20, Novelty |
| | Score <1.67 |
| Genomic Stability | Karyotype or KaryoStat ™ test: normal (diploid) |
| Morphology | Defined border, relatively round colonies, |
| | tightly packed cells with a high |
| | nucleus:cytoplasm ratio, phased-bright center |
| Doubling Time | 2D: <24 hours |
| Microbiological | Negative/None detected |
| sterility | |
| Identity | STR profiling and HLA typing (important |
| | for Immunogenicity Studies) |
| TCR sequencing | Confirms monoclonality of TCR |

SFFF iHSC Redifferentiation Protocol: Success criteria were set for the purity and fold expansion of differentiated iHSCs and iγδ T cells based on results from a previously established two-step, 2D research-scale SFFF redifferentiation protocol. Exemplary success criteria include, for example, TS T-iPSC to iHSC redifferentiation having a purity greater than 70% Lin-CD34+ cells and a fold expansion/input iPSC greater than 100 for 2D cultures and greater than 0.5 for 3D/2D cultures, and iHSC to iγδ T cell differentiation having a purity of 20% to 40% CD3+TCR γδ+ cells and a fold expansion/input iCD34 greater than 5. The protocol for the first redifferentiation step (iPSC to iHSC) was optimized using two TS T-iPSC clones, clones 1 and 2. Clone 2 is a subclone of clone 1. Clone 3 was used to validate the optimized protocol. A flow diagram of the 2D SFFF redifferentiation protocol is shown in FIG. 2. On Days 6-10, nonadherent cells harvested with medium change are added back to the culture. Flow cytometry was used to measure purity and viability of iHSCs (defined as Lin-CD34+ cells) as shown in FIGS. 6A-6B.

SFFF iγδ T Cell Redifferentiation Protocol: The previously established 2D protocol for the second redifferentiation step (iHSC to iγδ T cell) was used to measure the T cell redifferentiation potential of the iHSCs generated in the first redifferentiation step of the optimized protocol. Culture conditions of the iHSC to iγδ T cell redifferentiation protocol were as follows: the iHSCs were cultured in a medium containing StemSpan™ Lymphoid Progenitor Expansion Medium containing SCF, TPO, Flt3L, and IL-7. The medium was changed on Days 13, 17, and 20. The iHSCs were redifferentiated on VCAM1+DLL4-coated tissue culture plates. Further, the iHSC to iγδ T cell redifferentiation was performed under normoxic conditions (18% O₂). Flow cytometry was used to measure the purity and viability of iγδ T cells (defined as CD3+ Vδ9+Vδ2+ cells) as shown in FIG. 6C.

### Results

γδ T cells were selected as the iPSC cell of origin because it is known that the epigenetic and transcriptional memory of the starting cell influences and enhances iPSC redifferentiation potential to that cell type.⁴ γδ T-iPSCs underwent detailed characterization using assays that evaluated pluripotency, genomic stability, morphology, doubling time, sterility, identity, and TCR monoclonality. Table 6 lists the assays used to characterize the iPSC clones and the acceptance criteria for each assay.

The previously established protocol for the first redifferentiation step (iPSC to iHSC) was optimized using two γδ T-iPSC clones, clones 1 and 2, with the goal of increasing iHSC purity, fold expansion and T cell redifferentiation potential. FIG. 6A-6C show the results of this optimization. Morphological changes in the iPSC colonies during hematopoietic redifferentiation are shown in FIG. 6A, while the purity and fold expansion of iHSCs in the adherent and nonadherent fractions are summarized in FIG. 6B for two to three independent experiments. Notably, the purity and fold expansion of iHSCs redifferentiated using the optimized protocol exceeded the success criteria defined for this redifferentiation step, in an unexpected and surprising magnitude.

To determine whether the optimized SFFF iHSC redifferentiation protocol also increased the T cell redifferentiation potential of the redifferentiated iHSCs, a previously established SFFF iγδ T cell redifferentiation protocol was used to redifferentiate the iHSCs from the adherent fraction into iγδ T cells. As shown in FIG. 6C, the purity and fold expansion of the redifferentiated iγδ T cells unexpectedly and surprisingly exceeded the success criteria defined for this redifferentiation step. Together, these data show that the changes to the SFFF iHSC redifferentiation protocol increased not only iHSC purity and fold expansion but also their T cell redifferentiation potential.

Given that iPSC clones are heterogeneous in their T cell redifferentiation potential⁵, it is important to validate the optimized γδ T-iPSC to iHSC redifferentiation protocol using multiple iPSC clones. To this end, the optimized protocol has been validated using other fully characterized clones. The data for clone 3 are shown in FIG. 6A-6C. Similar morphological changes were observed during hematopoietic redifferentiation among the three clones (FIG. 6A). Also, even though iHSC purity from clone 3 approached the defined criterion of 70%, its iHSC fold expansion met or exceeded the defined criterion of 100-fold (FIG. 6B). Importantly, when the T cell redifferentiation potential of the clone 3-derived iHSCs from the adherent fraction was evaluated, it was found that the iγδ T cell purity and fold expansion also unexpectedly and surprisingly exceeded the defined criteria (FIG. 6C). Therefore, unexpectedly superior enhanced T cell redifferentiation potential was observed for two γδ T-iPSC subclones and two individual γδ T-iPSC clones, demonstrating the efficiency and validity of the optimized iHSC redifferentiation protocol.

### Example 2: Protocol for Redifferentiation of δδ T-iPSC Clones to i γδ T Cells

### Materials

TABLE 7
Reagents and supplies for cell culture and differentiation

| | Product No. |
|---|---|
| Item | (Exemplary) |
| γδT-iPSC clones | Academic Institute, Research |
| | Institute |
| iMatrix 511 | Amsbio, cat. # AMS.892 012 |
| StemFit Basic 04 | Amsbio, cat. # SFB-504-CT |
| bFGF | Amsbio, cat. # AMS.AS-bFGF |
| Y-27632 - ROCK Inhibitor | StemCell Technologies, cat# |
| | 72304 |
| TryPLE Select 1X | Thermofisher, cat. # 12563011 |
| 1X DPBS | Thermofisher, cat. # 14190144 |
| CryoStor CS10 | StemCell Technologies, cat. # |
| | 07930 |
| 6 well tissue coated treated plate | |
| 10 mL individual wrapped | |
| Serological Pipets | |
| 5 mL individual wrapped | |
| Serological Pipets | |
| 2 mL individual wrapped | |
| Serological Pipets | |
| P1000 sterile tips | |
| P10 sterile tips | |
| 15 mL conical tubes | |
| 50 mL conical tubes | |
| 1 mL cryovials | |
| 1.5 mL Eppendorf tube | |
| Cell lifter | |
| CoolCell Freezing container | |
| NucleoCounter NC-200 | |
| Orflo Moxi Cyte Viability Reagent | Cat. # MXA055 |
| Moxi GO II/Moxi V/Moxi Flow | Cat. # MXC030 |
| Cassettes, Type S+, 25 Pack | |
| (50 Tests) | |

### Exemplary Procedure

1.2 Supplemented StemFit Basic 04 Medium was prepared as follows:
   a. The StemFit Basic 04 medium was provided frozen. StemFit medium should be stored at below −20° C. until use. Sterile techniques were used to prepare Supplemented StemFit medium.
   b. Frozen StemFit Basic 04 medium was thawed with occasional mixing at room temperature (15-25° C.) or in a refrigerator (2-8° C.).
   c. Medium was not thawed at 37° C., as it accelerates the degradation of the medium ingredients.
   d. StemFit Basic 04 medium was supplemented with an additional 20 ng/mL of bFGF to reach a final concentration of 100 ng/mL bFGF of complete medium.
1.3 One 6-well tissue culture plate with iMatrix 511 was prepared as follows:
   a. 12 mL of 1×PBS were pipetted into a 15 mL conical tube.
   b. 30 μL of iMatrix 511 were added to 1×PBS and mixed well immediately (coating concentration: 0.25 μg/cm²).
   c. 2 mL of iMatrix solution were added to each well.
   d. The plates were incubated at 37° C., 5% CO₂, for at least 60 minutes (plates can be left overnight at 37° C.).
   e. Note: Plates can be stored at 4° C. for one week.
1.4 Supplemented StemFit® Basic 04 medium with 10 μm Y-27632 ROCK inhibitor was prepared as follows:
   a. The medium was thawed prior to use.
   b. 25 mL of supplemented Stemfit Basic 04 100 ng/mL bFGF were aliquoted into a 50 mL conical tube.
   c. 10 μm Y-27632 ROCK inhibitor were added.
1.5 A γδT-iPSC vial was thawed as follows:
   a. A frozen vial of γδT-iPSCs was thawed in a 37° C. water bath for 1 minute or until small pieces of frozen floating cells were visible.
   b. The thawed γδT-iPSCs were transferred from the vial slowly in a drop-wise manner into a 15 mL conical tube containing 9 mL of room temperature Supplemented StemFit 04 Basic medium with 10 μm Y-27632 ROCK inhibitor (Step 1.4).
   c. Note: Total volume can be 10 mL.
1.6 Cells were counted as follows:
   a. NucleoCounter NC-200 was used for cell counting purposes.
   b. 150 μL aliquot from cell suspension were removed (Step 1.5.b) to 1.5 mL Eppendorf tube.
   c. Vial-Cassette was inserted in the cell suspension from eppendorf tube and cells in cassette were collected.
   d. Vial-Cassette was inserted into NC-200 cell counter and cells were counted.
   e. Only the total number of live cells were considered for cell seeding purposes.
1.7 Freezing medium was removed as follows:
   a. The conical tube was centrifuged at 300×g for 3 minutes at room temperature.
   b. Supernatant was aspirated without disturbing the cell pellet by leaving behind 0.5 mL of Supplemented StemFit Basic 04 medium.
   c. Conical tube was gently tapped to loosen pellet.
   d. 1 mL of Supplemented StemFit Basic 04 medium (Step 1.4) was added with P1000.
   e. Additional Supplemented StemFit Basic 04 medium was added to get a final cell concentration of 1×10⁴ cells/mL.
   f. iMatrix 511 solution was removed from the 6-well plate by aspiration.
      i. Note: leaving plate to dry out for extended period (e.g., more than 10 minutes) was avoided. Wells should not be dried.
   g. The cells were thoroughly mixed and 2 mL/well were plated.
   h. The cells were distributed evenly by rocking the plate back and forth and side to side.
   i. The plate was overnight at 37° C. with 5% CO₂.
1.8 Day 1: The medium was changed as follows:
   a. Basic 04 medium containing 100 ng/mL bFGF was brought to room temperature.
   b. The medium from 6-well plate was aspirated.
   c. 2 mL/well of fresh medium (without ROCK inhibitor) were added to 6-well plate.
   d. Imaging of the plate was performed with EVOS7000 and recorded at 4× and 10× magnification.
   e. Three images were taken to record the morphology and landscape of the culture. A 10× magnification of exemplary cells is shown in FIG. 8P.
1.9 Culturing of the cells was continued until the cells reached typical morphology, exhibiting all iPSC characteristics including circular colonies, with densely packed cells, higher nucleus to cytoplasmic ratio, well defined edges, and distinct border. iPSC colonies were at ˜70-80% confluency at this stage.
   a. Note this could take 7 days+/−1 day.
   b. iPSC had defined borders and compact cells as shown in FIG. 8Q at 10× magnification.
1.10 Cells were passaged and pluripotency was characterized by flow cytometry before initiating redifferentiation
   a. iMatrix 6-well plates were prepared as in Step 1.3.
   b. Medium from wells was aspirated.
   c. Wells were rinsed with 1 mL 1×PBS.
   d. PBS was aspirated from wells.
   e. 1 mL/well TrypLE was added.
   f. Plates were incubated at 37° C. for 5-7 minutes.
   g. Wells were examined under microscope.
      i. Cells became bright and cell borders appeared as shown in FIG. 8R.
   h. TrypLE was gently removed.
   i. 1 mL of 20 ng/mL of bFGF was added and 10 μm Y-27632 ROCK inhibitor supplemented StemFit Basic 04 medium/well with P1000 and pipetted to remove cells. Bubbles were avoided.
   j. A microscope was used to ensure removal of 95% cells from wells.
      i. If residual cells were still adherent, 1 mL of 20 ng/mL of bFGF was added and 10 μm Y-27632 ROCK inhibitor supplemented StemFit Basic 04 medium. A cell lifter was used to scrape the well, and pipetted with P1000 three (3) times to ensure uniform population.
   k. Wells were collected into a 15 mL conical tube.
   l. Cell count was performed as in Step 1.6g.
   m. 1×10⁶ cells were collected for pluripotency flow analysis.
   n. 2×10³ cells/well were plated with Stemfit medium containing 100 ng/mL bFGF and 10 μm ROCK inhibitor (Step 1.4).
      i. Depending on cell number, enough volume was prepared to expand cells to required numbers of 6-well plates.
      ii. If there were excess cells, then Cryostor CS10 was used for freezing remaining cells.
1.11 Existing medium was replaced with StemFit Basic 04 medium containing 100 ng/mL bFGF after 24 hours.
1.12 Cells were continued to be incubated and cultured at 37° C., 5% CO₂ and medium was changed every alternative day until Day 0.

### Day 0: Onset of Hematopoietic Differentiation—Mesoderm Induction Phase

2.1 STEP 1 medium for Days 0-2 was prepared as follows:

| | | Product | Stock | | Final | | Volume for | |
|---|---|---|---|---|---|---|---|---|
| Reagent | Manufacturer | No. | Concentration | | Concentration | | 6 well plate | |
| StemFit | AMS Bio | SFB- | | | | | 12 | mL |
| Basic 04 | | 504-CT | | | | | | |
| complete | | | | | | | | |
| CHIR99021 | TOCRIS | 4423 | 20 | mM | 4 | μM | 2.4 | μL |
| rh BMP4 | R&D | 314-BP | 100 | μg/mL | 80 | ng/mL | 9.6 | μL |
| rh VEGF | R&D | 293-VE | 100 | μg/mL | 80 | ng/mL | 9.6 | μL |

2.2 The plate was taken out from incubator and observed under inverted phase contrast microscope for cell growth and morphology.
   a. Images were recorded at Day 0. A 4× magnification of exemplary cells is shown in FIG. 8S, and a 10× magnification of exemplary cells is shown in FIG. 8T.
2.3 Medium was carefully aspirated and 2 mL/well STEP 1 medium was replaced in all wells without any wash or rinse.
2.4 Cells were continued to be incubated and cultured at 37° C., 5% CO₂.

### Day 2: Onset of Hematopoietic Differentiation—Mesoderm to Definitive Hematopoietic Lineage Specification

2.5 At Day 2, STEP 2 medium for Days 2-4 was prepared as follows:

| | | Product | Stock | | Final | | Volume for | |
|---|---|---|---|---|---|---|---|---|
| Reagent | Manufacturer | No. | Concentration | | Concentration | | 6 well plate | |
| Advanced | Gibco | 12634-10 | 100% | | | | 9.6 | mL |
| DMEM/F12 | | | | | | | | |
| StemFit ® For | Ajinomoto | 20170228A | 100% | | 20% | | 2.4 | mL |
| Differentiation | | | | | | | | |
| L-Glutamine | Gibco | 25030-081 | 100X | | 1X | | 120 | μL |
| P/S | Gibco | 15140-122 | 100X | | 0.5X-1X | | 60 | μL |
| SB431542 | Fujifilm Wako | 033-24631 | 5 | mM | 1.5-2.5 | μM | 4.8 | μL |
| | pure chemical | | | | | | | |
| | corporation | | | | | | | |
| rh VEGF | R&D | 293-VE | 100 | μg/mL | 80 | ng/mL | 9.6 | μL |
| bFGF | Wako | 060-04543 | 100 | μg/mL | 50 | ng/mL | 6.0 | μL |
| SCF | R&D | 255-SC | 100 | μg/mL | 50 | ng/mL | 6.0 | μL |

2.6 The plate was taken out from incubator and observed under inverted phase contrast microscope for cell growth and morphology.
   a. Images were recorded at Day 2 as in Step 2.2a. A 4× magnification of exemplary cells is shown in FIG. 8U, and a COX magnification of exemplary cells is shown in FIG. 8V.
2.7 The medium was carefully aspirated and 2 mL/well STEP 2 medium was replaced in all wells without any wash or rinse.
2.8 Cells were continued to be incubated and cultured at 37° C., 5% CO₂.

### Day 4: Onset of Hematopoietic Differentiation—Hemanginoblast Induction Phase

2.9 At Day 4, STEP 3 medium for Day 4-6 was prepared as follows:

| | | Product | Stock | Final | Volume for | |
|---|---|---|---|---|---|---|
| Reagent | Manufacturer | No. | Concentration | Concentration | 6 well plate | |
| Advanced | Gibco | 12634-10 | 100% | | 9.6 | mL |
| DMEM/F12 | | | | | | |
| StemFit ® For | Ajinomoto | 20170228A | 100% | 20% | 2.4 | mL |
| Differentiation | | | | | | |
| L-Glutamine | Gibco | 25030-081 | 100X | 1X | 120 | μL |
| P/S | Gibco | 15140-122 | 100X | 0.5X-1X | 60 | μL |
| SCF | R&D | 255-SC | 100 μg/mL | 50 ng/mL | 6.0 | μL |
| rh VEGF | R&D | 293-VE | 100 μg/mL | 20 ng/mL | 2.4 | μL |
| IL-3 | Peprotech | AF-200-03 | 100 μg/mL | 50 ng/mL | 6.0 | μL |
| IL-6 | R&D | 206-IL-050 | 100 μg/mL | 50 ng/mL | 6.0 | μL |
| Flt3L | R&D | 308-FK-025 | 100 μg/mL | 50 ng/mL | 6.0 | μL |
| EPO | R&D | 287-TC-500 | 500 IU/mL | 10 IU/mL | 240 | μL |

2.10 The plate was taken out from incubator and observed under inverted phase contrast microscope for cell growth and morphology.
   a. Images were recorded at Day 4 as in Step 2.2a. A 4× magnification of exemplary cells is shown in FIG. 8X, and a 10× magnification of exemplary cells is shown in FIG. 8Y.
2.11 The medium was carefully aspirated and 2 mL/well STEP 3 medium was replaced in all wells without any wash or rinse.
2.12 Cells were continued to be incubated and cultured at 37° C., 5% CO₂.

### Day 6: Onset of Hematopoietic Differentiation—Endothelial to Hematopoietic Transition Phase

2.13 At Day 6, STEP 4 medium for Day 6-10 was prepared as follows:

| | | Product | Stock | Final | Volume for | |
|---|---|---|---|---|---|---|
| Reagent | Manufacturer | No. | Concentration | Concentration | 6 well plate | |
| Advanced | Gibco | 12634-10 | 100% | | 9.6 | mL |
| DMEM/F12 | | | | | | |
| StemFit ® For | Ajinomoto | 20170228A | 100% | 20% | 2.4 | mL |
| Differentiation | | | | | | |
| L-Glutamine | Gibco | 25030-081 | 100X | 1X | 120 | μL |
| P/S | Gibco | 15140-122 | 100X | 0.5X-1X | 60 | μL |
| SCF | R&D | 255-SC | 100 μg/mL | 50 ng/mL | 6.0 | μL |
| IL-6 | R&D | 206-IL-050 | 100 μg/mL | 50 ng/mL | 6.0 | μL |
| EPO | R&D | 287-TC-500 | 500 IU/mL | 10 IU/mL | 240 | μL |

2.14 The plate was taken out from incubator and observed under inverted phase contrast microscope for cell growth and morphology.
   a. Images were recorded at Day 6 as in 2.2a. A 4× magnification of exemplary cells is shown in FIG. 8Z, and a 10× magnification of exemplary cells is shown in FIG. 8AA.
2.15 The medium was carefully aspirated and 2 mL/well STEP 4 medium was replaced in all wells without any wash or rinse.
2.16 Cells were continued to be incubated and cultured at 37° C., 5% CO₂.
2.17 At Day 8, STEP 4 media was prepared as table above.
2.18 The plate was taken out from incubator and observed under inverted phase contrast microscope for cell growth and morphology.
   a. Images were recorded at Day 8 as in Step 2.2a. A 4× magnification of exemplary cells is shown in FIG. 8BB, and a 10× magnification of exemplary cells is shown in FIG. 8CC.
2.19 The medium was collected from all wells of the plate in 15 mL conical tube.
2.20 1 mL Step 4 medium was replaced in each well.
2.21 15 mL conical tube containing cells and medium was centrifuged at 300×g for 5 minutes at room temperature.
2.22 Supernatant was discarded and cell pellet was resuspended in 6 mL fresh STEP 4 medium and 1 mL was distributed to each well to make final volume 2 mL per well.
2.23 Cells were continued to be incubated and cultured at 37° C., 5% CO₂.
   Day 10: Hematopoietic Cells (iCD34/iHSCs) were Harvested and Samples were Prepared for Flow Cytometric Characterization and Cryofreezing of iCD34/iHSCs
2.24 The plate was taken out from incubator and observed under inverted phase contrast microscope for cell growth and morphology.
   a. Images were recorded at Day 10 as in Step 2.2a. A 4× magnification of exemplary cells is shown in FIG. 8DD, and a 10× magnification of exemplary cells is shown in FIG. 8EE.
2.25 Non adherent fraction was processed as follows: the floating cells were collected by gently swirling the plate into new 50 mL conical tube.
2.26 The conical tube was centrifuged at 300×g for 5 minutes at room temperatures.
2.27 The supernatant was discarded and cells were resuspended in the STEP 4 medium without cytokines or growth factors and the cells were counted.
2.28 Cell Count was performed using NC-200.
2.29 Adherent cell fraction was processed as follows: Using STEP 4 medium without cytokine and growth factors, adherent cell fraction was resuspended to detach adherent cells from the plate (4-6 times mixing using P1000).
2.30 The cells were collected into new 50 mL conical tube and resuspended gently with 10 mL pipette and passed through 70 μm cell strainer followed by 40 μm cell strainer.
2.31 An appropriate number of cells were used for flow cytometric analysis using the iHSC panel.
2.32 iHSCs/iCD34 cells were cryopreserved using CS-10 freezing medium.
2.33 Cryopreserving method: cells were centrifuged at 300×g for 5 minutes.
2.34 Supernatant was removed.
   a. Cell pellet was resuspended in CS-10 at 1-2×10⁶ cells/mL and frozen into cryovials.
   b. Cryovials were placed into CoolCells and store containers in −80° C. freezer.
   c. Cells were transferred the following day to Liquid N₂ tank for storage.
      Day 10: ICD34/iHSCs to iγδ T Cell Differentiation Phase
3.1 VCAM1/DLL1/4/Jagged 2 coated plates were prepared.
3.2 10 μg/mL of DLL4 or DLL1 or Jagged 2 or all three Notch ligand proteins+5 μg/mL of VCAM1 were prepared in PBS (without Ca⁺² or Mg⁺²).
3.3 100 μL/well were added to tissue culture treated 48 well plate and it was shaken well to cover the surface of the well.
3.4 The plate was incubated at 4° C. overnight or 37° C. for one hour (PBS or water was added into well around the coated wells to avoid evaporation).
3.5 Harvested iCD34/iHSCs were resuspended into Lymphoid Progenitor Expansion Medium (StemCell Technologies).
3.6 Cell count was performed.
3.7 From the pre-coated plate, VCAM1/DLL1/4/Jagged 2/PBS was removed and the plate washed with PBS (without Ca⁺² or Mg⁺²) once.
3.8 1.2×10⁴ live cells/250 μL of Lymphoid Progenitor Expansion Medium were seeded per well of the pre-coated 48 well plate.
3.9 Incubated at 37° C. 5% CO₂, 18% O₂ for further culture and differentiation.
3.10 Day 13: 250 μL of Lymphoid Progenitor Expansion Medium were added.
3.11 Day 17: 250 μL of the medium were removed and 270 μL* of Lymphoid Progenitor Expansion medium were added.
3.12 Day 20: Without disturbing cells on the plate, 250 μL of the medium was removed and 270 μL* of Lymphoid Progenitor Expansion Medium was added until Day 24.
3.13 Day 24: The cells were harvested.
3.14 The cells were counted and samples were prepared for flow cytometric analysis to assess purity of iγδ T cells. (* intended to be half-medium change, but considering evaporation, a little more than removed volume was added.)

### Example 3: Serum-Free, Feeder-Free, 3D/2D Redifferentiation of γδ T Cell-Derived iPSCs into iHSCs and then into iγδ T Cells

### Materials

TABLE 8
Reagents for iPSC culture and maintenance.

| Item | Product No. |
|---|---|
| γδ T-iPSC clones | Academic Institution, Contract |
| | Research Organization |
| iMatrix 511 | Amsbio, cat. # AMS.892 012 |
| StemFit ® Basic 04 Complete | Amsbio, cat. # SFB-504-CT |
| rh bFGF | Amsbio, cat. # AMS-FGF-100 |
| Y-27632 - ROCK Inhibitor, 1 mg | StemCell Technologies, cat# 72302 |
| TryPLE ™ Select 1X | ThermoFisher, cat. # 12563011 |
| Collagenase Type II | StemCell Technologies, cat. # 07418 |
| 1X DPBS | ThermoFisher, cat. # 14190144 |
| CryoStor CS10 | StemCell Technologies, cat. # 07930 |

TABLE 9
Reagents needed for hematopoietic differentiation
(listed by day of redifferentiation).

| | |
|---|---|
| Reagent | Product No. |

Day 0 Reagents

| | |
|---|---|
| StemFit Basic 04 Complete | Amsbio, cat. # SFB-504-CT |
| CHIR99021 | TOCRIS, cat. # 4423 |
| rh BMP4 | R&D, cat. # 314-BP |
| rh VEGF | R&D, cat. # 293-VE |

Day 2 Reagents

| | |
|---|---|
| Advanced DMEM/F12 | Gibco, cat. # 12634-10 |
| StemFit ® For Differentiation | Ajinomoto, cat. # 20170228A |
| L-Glutamine | Gibco, cat. # 25030-081 |
| Penicillin-Streptomycin P/S | Gibco, cat. # 15140-122 |
| SB431542 | Fujifilm Wako pure chemical |
| | corporation, cat. # 033-24631 |
| rh VEGF | R&D, cat. # 293-VE |
| rh bFGF | Amsbio, cat. # AMS-FGF-100 |
| rh SCF | R&D, cat. # 255-SC |

Day 4 Reagents

| | |
|---|---|
| Advanced DMEM/F12 | Gibco, cat. # 12634-10 |
| StemFit ® For Differentiation | Ajinomoto, cat. # 20170228A |
| L-Glutamine | Gibco, cat. # 25030-081 |
| P/S | Gibco, cat. # 15140-122 |
| rh SCF | R&D, cat. # 255-SC |
| rh VEGF | R&D, cat. # 293-VE |
| rh IL-3 | Peprotech, cat. # AF-200-03 |
| rh IL-6 | R&D, cat. # 206-IL-050 |
| rh Flt3L | R&D, cat. # 308-FK-025 |
| rh EPO | R&D, cat. # 287-TC-500 |

Days 6-10 Reagents

| | |
|---|---|
| Advanced DMEM/F12 | Gibco, cat. # 12634-10 |
| StemFit ® For Differentiation | Ajinomoto, cat. # 20170228A |
| L-Glutamine | Gibco, cat. # 25030-081 |
| P/S | Gibco, cat. # 15140-122 |
| rh SCF | R&D, cat. # 255-SC |
| rh IL-6 | R&D, cat. # 206-IL-050 |
| rh EPO | R&D, cat. # 287-TC-500 |

TABLE 10
Reagents for iγδ T cell redifferentiation.

| Reagent | Product No. |
|---|---|
| StemSpan ™ SFEM II | StemCell Technologies, cat. # 09655 |
| Lymphoid Progenitor Expansion | StemCell Technologies, cat. # 09915 |
| Supplement (10X) | |
| rh VCAM-1-Fc | R&D, cat. # 862-VC-100 |
| rh DLL4-Fc | R&D, cat. # 10185-D4-050 |

TABLE 11
Supplies and consumables needed for cell culture.

| Item | Product No. |
|---|---|
| Corning ™ Costar ™ 6-well Clear | Corning, cat. # 3516 |
| TC-treated Multiple Well Plates, | |
| Individually Wrapped, Sterile | |
| Corning ™ Costar ™ 48-well Clear | Corning, cat. # 3548 |
| TC-treated Multiple Well Plates, | |
| Individually Wrapped, Sterile | |
| Costar ® 6-well Clear Flat Bottom | Corning, cat. # 3471 |
| Ultra-Low Attachment Multiple | |
| Well Plates, Individually Wrapped, | |
| Sterile | |
| Fisherbrand ™ Sterile Polystyrene | ThermoFisher, |
| Disposable Serological Pipets with | cat. # 13-676-10H, |
| Magnifier Stripe (various sizes) | cat. # 13-678-11E, |
| | cat. # 13-678-11, |
| | cat. # 13-678-11F |
| Pipette Tips RT LTS 20 μL F 960A/10 | Rainin, cat. # 30389225 |
| Pipette Tips RT LTS 200 μL F 960A/10 | Rainin, cat. # 30389239 |
| Pipette Tips RT LTS 1000 μL F 768A/8 | Rainin, cat. # 30389212 |
| 50 mL conical tubes | Corning, cat. # 352098 |
| Falcon ® 15 mL Polystyrene Centrifuge | Corning, cat. # 352095 |
| Tube, Conical Bottom, with Dome Seal | |
| Screw Cap, Sterile | |
| 1.8 mL cryovials | Thermofisher, cat. # 375418 |
| 1.5 mL Eppendorf tubes | Eppendorf, cat. # 022363204 |
| Cell lifters | Corning, cat. # 3008 |
| Corning CoolCell ™ Freezing container | Corning, cat. # 432006 |
| Via-1-Cassette | Chemometec, cat. # 941-0012 |

TABLE 12
Antibodies for Flow Panels.

| | | | |
|---|---|---|---|
| Fluorochrome | Marker | Vendor | Cat# |

Pluripotency

| | | | |
|---|---|---|---|
| BV711 | SSEA-1 | BioLegend | 323050 |
| PE-Cy7 | SSEA-3 | BioLegend | 330326 |
| PerCP/Cy5.5 | CD34+ | BioLegend | 343522 |
| PE | SSEA-4 | BioLegend | 330406 |
| AF647 | OCT 3/4 | BioLegend | 653710 |
| BV421 | SOX2 | BioLegend | 656114 |

iHSC Panel

| | | | |
|---|---|---|---|
| PE | CD34 | BioLegend | 343506 |
| APC | CD43 | BD | 560198 |
| BUV395 | CD45 | BD | 563792 |
| BV421 | CD38 | BioLegend | 303526 |
| APC-Cy7 | Live/dead Near IR | ThermoFisher | L10119 |
| FITC | CD3 | BioLegend | 300306 |
| FITC | CD235a | BioLegend | 349104 |
| FITC | CD11c | BioLegend | 337214 |
| FITC | CD11b | BioLegend | 301330 |
| FITC | CD14 | BioLegend | 325604 |
| FITC | CD56 | BioLegend | 318304 |
| FITC | CD19 | BioLegend | 302206 |
| FITC | CD20 | BioLegend | 302304 |
| FITC | CD16 | BioLegend | 302006 |
| FITC | CD2 | BioLegend | 300206 |

iγδ T cell redifferentiation

| | | | |
|---|---|---|---|
| BUV395 | CD3 | BD | 740283 |
| BUV496 | CD25 | BD | 741144 |
| BUV563 | CD45RA | BD | 612926 |
| BUV615 | CD2 | BD | 751450 |
| BUV661 | CD277(BTN3) | BD | 750227 |
| BUV737 | CD56 | BD | 612766 |
| BUV805 | CD11a | BD | 748572 |
| BV421 | CD197(CCR7) | BioLegend | 353208 |
| BV480 | CD95 | BD | 746675 |
| BV605 | CD5 | BioLegend | 364020 |
| sBV650 | CD16 | BioLegend | 302042 |
| BV711 | HLA-ABC | BD | 565333 |
| BV786 | CD122 | BD | 743118 |
| FITC | Vγ9 TCR | BioLegend | 331306 |
| PerCP-Cy5.5, | CD215 | Invitrogen | 46-7149-82 |
| BB700 | (IL-15Rα) | | |
| PE | BTN2A1 | LSBio | LS-C649249-100 |
| PE-Texas Red | NKG2D | BD | 562498 |
| PE-Cy5 | CD1a | BioLegend | 300108 |
| PE-Cy7 | CD27 | BioLegend | 356412 |
| APC, Alexa 647 | Vδ2 TCR | BioLegend | 331418 |
| Alexa 700 | CD127 | BD | 565185 |
| APC-Cy7 | Live/dead Near IR | ThermoFisher | L10119 |

### Exemplary Procedure

1.1 Supplemented StemFit Basic 04 Complete Type Medium was prepared as follows:
   a. The StemFit Basic 04 Complete Type Medium was provided frozen and was stored at or below −20° C. until use. Sterile techniques were used to prepare Supplemented StemFit Basic 04 Complete Type Medium.
   b. Frozen StemFit Basic 04 Complete Type Medium was thawed with occasional mixing at room temperature (15-25° C.) or in a refrigerator (2-8° C.).
   c. Note: medium was not thawed at 37° C., as it accelerates the degradation of the medium ingredients.
   d. StemFit Basic 04 Complete Type Medium was supplemented with an additional 20 ng/mL of bFGF to reach 100 ng/mL bFGF in the supplemented medium.
   e. Supplemented medium was stored for up to two (2) weeks at 4° C.
   f. Medium was brought to room temperature (15-25° C.) for at least one (1) hour prior to use.
1.2 10 mM Y-27632 ROCK inhibitor stock solution was prepared as follows:
   a. Y-27632 ROCK inhibitor was received as a crystalline solid and stored at −20° C. for up to 12 months from date of receipt.
   b. 10 mM stock solution in 1×DPBS was prepared as follows:
      i. 312 μL of 1×DPBS were added to 1 mg Y-27632 and solution was resuspended completely.
   ii. 25 μL of stock solution were aliquoted into Eppendorf tubes labeled as Y-27632 stock.
   iii. Aliquots were stable at −20° C. for up to 6 months.
   iv. Working volumes were aliquoted to avoid repeated freeze-thaw cycles.
1.3 One 6-well tissue culture plate coated with iMatrix 511 was prepared as follows:
   a. 12 mL of 1×DPBS were pipetted into a 15 mL conical tube.
   b. 30 μL of iMatrix 511 were added to 1×DPBS and immediately mixed well (coating concentration: 0.25 μg/cm²).
   c. 2 mL of iMatrix solution were added to each well.
   d. Plates were incubated at 37° C., 5% CO₂, for at least 60 minutes (plates could be left overnight at 37° C.).
   e. Note: After coating step, plates could be stored at 4° C. for one week. Prior to use, plate was incubated at 37° C. for one hour.
1.4 Supplemented StemFit Basic 04 Complete Type Medium (Step 1.1) with 10 μM Y-27632 ROCK inhibitor was prepared as follows:
   a. An aliquot of 10 mM Y-27632 ROCK inhibitor stock solution was thawed prior to use.
   b. 25 mL of Supplemented StemFit Basic 04 Complete Type Medium (Step 1.1) was aliquoted into a 50 mL conical tube.
   c. 25 μL of 10 mM Y-27632 ROCK inhibitor was added to Supplemented StemFit Basic 04 Complete Type Medium to achieve a final concentration of medium with 10 μM Y-27632 ROCK inhibitor.
1.5 γδ T-iPSC vial was thawed as follows:
   a. A vial of frozen γδ T-iPSCs was thawed in a 37° C. water bath for 1 minute or until small pieces of frozen floating cells were visible.
   b. The thawed γδ T-iPSCs were transferred from the vial slowly in a drop-wise manner into a 15 mL conical tube containing 9 mL of room temperature Supplemented StemFit 04 Basic Complete Type Medium with 10 μm Y-27632 ROCK inhibitor (from Step 1.4).
   c. Note: Total volume was 10 mL.
1.6 Cell count was performed as follows:
   a. The NucleoCounter NC-200 instrument was used for cell counting purposes.
   b. 150 μL aliquot was removed from the cell suspension (Step 1.5.b) to a 1.5 mL Eppendorf tube.
   c. Cells were collected from Eppendorf tube into a Vial-Cassette.
   d. Vial-Cassette was inserted into the NC-200 cell counter and cells were counted.
   e. Note: Only total number of live cells were used for cell seeding purposes
1.7 Washing and plating thawed γδ T-iPSCs were performed as follows:
   a. 15 mL conical tube containing thawed γδ T-iPSCs (Step 1.5 b) was centrifuged at 300×g for 3 minutes at room temperature.
   b. Supernatant was aspirated without disturbing the cell pellet by leaving behind 0.5 mL of medium.
   c. Conical tube was gently tapped to loosen cell pellet.
   d. 1 mL of Supplemented StemFit Basic 04 Complete Type Medium with 10 μM Y-27632 ROCK inhibitor (Step 1.4) was added with P1000 to resuspend cells.
   e. Additional Supplemented StemFit Basic 04 Complete Type Medium with 10 μM Y-27632 ROCK inhibitor (Step 1.4) was added to achieve a final cell concentration of 1×10⁴ cells/mL.
   f. iMatrix 511 solution was removed from the 6-well plate by aspiration.
      i. Note: the plate was not left to dry out for extended period (e.g., more than 10 minutes). Wells were not allowed to dry.
   g. Cells were mixed thoroughly and then 2 mL/well were plated.
   h. Cells were distributed evenly on the bottom of the well by manually rocking the plate gently back and forth and side to side.
   i. Plate was incubated overnight at 37° C. in 5% CO₂.
1.8 Day 1: Medium change was performed as follows:
   a. Supplemented StemFit Basic 04 Complete Type Medium (Step 1.1) was brought from 4° C. to room temperature.
   b. Medium was gently aspirated from 6-well plate.
   c. 2 mL/well of fresh StemFit Basic 04 Complete Type Medium (without ROCK inhibitor)(Step 1.1) was added to 6-well plate.
   d. Imaging of the plate was performed with EVOS M7000 and 4× and 10× magnification were recorded.
   e. Three images of different regions on the plate were taken to record the morphology and landscape of the culture. There were single cells and two-celled colonies present. Cells had a spiky morphology due to the ROCK inhibitor.
      i. Note: as cells divide, borders became more defined.
      ii. An example view at 10× magnification is shown in FIG. 8FF.
1.9 Cells continued to be cultured until they reached typical morphology, exhibiting all iPSC characteristics including circular colonies with densely packed cells, high nucleus to cytoplasmic ratio, well-defined edges, and distinct borders. iPSC colonies were at ˜70-80% confluency at this stage.
   a. Note: It could take 7 days+/−1 day for the colonies to exhibit this morphology.
   b. iPSC colonies had defined borders and compact cells.
      i. An example view at 10× magnification is shown in FIG. 8GG.
1.10 Cells were passaged and pluripotency was confirmed by flow cytometry before initiating redifferentiation
   a. Passaging iPSCs was performed as follows:
      i. Medium was aspirated from cells plated in Step 1.7.
      ii. Wells were rinsed with 1 mL 1×DPBS.
      iii. 1×DPBS was aspirated from wells.
      iv. 1 mL TrypLE was added and then incubated at 37° C. for 6-8 minutes.
      v. Wells were examined under a microscope. Cells appeared bright with clear cell borders as shown in FIG. 8HH.
      vi. TrypLE was gently removed.
      vii. 1 mL of Supplemented StemFit Basic 04 Complete Type Medium with 10 μM Y-27632 ROCK inhibitor (from Step 1.4) was added with P1000 and then pipetted gently to remove cells from plate. Forming bubbles was avoided.
   b. Wells were examined with microscope to ensure removal of 95% of the cells from wells.
   c. If residual cells were still adherent to the plate, then 1 mL of StemFit Basic 04 Complete Type Medium with 10 μM Y-27632 ROCK inhibitor (Step 1.4) was added. A cell lifter was then used to scrape the well, and a P1000 was used to pipette three (3) times to ensure uniformed, single cell suspension.
   d. Cells were collected into a 15 mL conical tube.
   e. Cell count was performed as in Step 1.6.
   f. 1×10⁶ cells were removed and used for pluripotency flow analysis.
   g. Pluripotency flow panel is shown in Table 12.
   h. Acceptance criteria were met before proceeding to redifferentiation steps. Acceptance criteria for pluripotency are:
      >85% Oct4+, Sox2+, SSEA3+, SSEA4+
      <1% SSEA1+
      <5% CD34+
   i. Day −2: Redifferentiation of γδ T cell-derived iPSCs into iγδ T cells: Based on cell count (Step 1.10.e), 0.6×10⁶ cells/well were plated with Supplemented StemFit Basic 04 Complete Type Medium with 10 μM Y-27632 ROCK inhibitor (Step 1.4) in a low-attachment 6-well plate.
      i. Note: iMatrix-511 coating was not required.
      ii. Note: iPSCs were passaged at least 5 times prior to initiating redifferentiation. Continued maintenance of iPSCs from Steps 1.10.a-h.
      iii. Depending on cell count (Step 1.10.e), 3.6×10⁶ cells were prepared in 24 mL Supplemented StemFit Basic 04 Complete Type Medium with 10 μM Y-27632 ROCK inhibitor (Step 1.4) and 4 mL/well were distributed in a low-attachment 6-well plate.
      iv. If there were any cells remaining after plating, they were cryopreserved in Cryostor CS-10.
         a. Cells were cryopreserved in CS-10 freezing medium as follows:
         i. Cells were centrifuged at 300×g for 5 minutes at room temperature.
         ii. Supernatant was carefully removed.
         iii. Cell pellet was suspended in CS-10 medium at 1-2×10⁶ cells/mL and aliquoted at 1 mL/cryovial.
         iv. Cryovials were placed into CoolCell freezing container and then placed into a −80° C. freezer overnight.
         v. Within 3 days, cells were transferred to Liquid N₂ tank for storage.
   j. The low-attachment plate was examined under a microscope to ensure that cells were evenly distributed among the wells.
   k. The plate was incubated in a shaker incubator/multitron and cells were cultured at 37° C., 5% CO₂ with 85% humidity for 48 hours. The plates were agitated at 100 RPM. Uniform embryoid bodies (EBs) were observed in 48 hours.
1.11 Day 0: Onset of hematopoietic differentiation—Mesoderm induction phase
   a. StemFit Basic 04 Complete Type Medium (Step 1.1) was pre-warmed at room temperature, and STEP 1 medium for Days 0-2 was prepared:

| | Stock | | Final | | Volume for | |
|---|---|---|---|---|---|---|
| Reagent | Concentration | | Concentration | | 6-well plate | |
| StemFit Basic 04 | | | | | 12 | mL |
| Complete Type Medium | | | | | | |
| (Step 1.1) | | | | | | |
| CHIR99021 | 20 | mM | 4 | μM | 2.4 | μL |
| rh BMP4 | 100 | μg/mL | 80 | ng/mL | 9.6 | μL |
| rh VEGF | 100 | μg/mL | 80 | ng/mL | 9.6 | μL |

b. The plate was taken from the incubator and placed on an inverted phase contrast microscope to observe cell growth and morphology.
c. Images were recorded at Day 0. A 4× magnification of exemplary cells is shown in FIG. 8II.
d. EBs including medium were collected from all wells of the low attachment plate and placed into a 50 mL conical tube.
e. 50 mL conical tube was centrifuged at 500×g for 3 minutes at room temperature.
f. The supernatant was carefully aspirated and pelleted EBs were gently resuspended in 12 mL fresh STEP 1 medium (Step 1.11.a) and 2 mL were distributed to each well of 6-well TC-treated plate.
g. Cells continued to be incubated and cultured at 37° C., 5% CO₂. No agitation was required at this stage.
1.12 Day 2: Onset of hematopoietic differentiation—Mesoderm to definitive hematopoietic lineage specification
   a. At Day 2, Advanced DMEM/F12 medium was pre-warmed at room temperature, and STEP 2 medium for Days 2-4 was prepared:

| | Stock | | Final | | Volume for | |
|---|---|---|---|---|---|---|
| Reagent | Concentration | | Concentration | | 6-well plate | |
| Advanced DMEM/F12 | 100% | | | | 9.6 | mL |
| StemFit ® For | 100% | | 20% | | 2.4 | mL |
| Differentiation | | | | | | |
| L-Glutamine | 100X | | 1X | | 120 | μL |
| P/S | 100X | | 0.5X-1X | | 60 | μL |
| SB431542 | 5 | mM | 1.5-2.5 | μM | 4.8 | μL |
| rh VEGF | 100 | μg/mL | 80 | ng/mL | 9.6 | μL |
| bFGF | 100 | μg/mL | 50 | ng/mL | 6.0 | μL |
| SCF | 100 | μg/mL | 50 | ng/mL | 6.0 | μL |

b. The plate was taken from the incubator and placed on an inverted phase contrast microscope to observe cell growth and morphology.
c. Images were recorded at Day 2 as in Step 1.11.c. A 4× magnification of exemplary cells is shown in FIG. 8JJ.
d. EBs and cells in medium were collected from all wells of the 6-well TC-treated plate and placed into a 15 mL conical tube.
e. 1 mL Step 2 medium (Step 1.12.a) was added to each well.
f. 15 mL conical tube containing EBs, cells, and medium was centrifuged at 500×g for 3 minutes at room temperature.
g. The supernatant was carefully aspirated and pelleted EBs and cells were gently resuspended in 6 mL fresh STEP 2 medium (Step 1.12.a), and 1 mL was distributed to each well so that final volume per well of the 6-well TC-treated plate was 2 mL.
h. Cells continued to be incubated and cultured at 37° C., 5% CO₂. No agitation was required at this stage.
1.13 Day 4: Onset of hematopoietic differentiation—Hemangioblast induction phase
   a. At Day 4, Advanced DMEM/F12 medium was pre-warmed at room temperature, and STEP 3 medium for Days 4-6 was prepared:

| | Stock | Final | Volume for | |
|---|---|---|---|---|
| Reagent | Concentration | Concentration | 6-well plate | |
| Advanced DMEM/F12 | 100% | | 9.6 | mL |
| StemFit ® For | 100% | 20% | 2.4 | mL |
| Differentiation | | | | |
| L-Glutamine | 100X | 1X | 120 | μL |
| P/S | 100X | 0.5X-1X | 60 | μL |
| SCF | 100 μg/mL | 50 ng/mL | 6.0 | μL |
| rh VEGF | 100 μg/mL | 20 ng/mL | 2.4 | μL |
| IL-3 | 100 μg/mL | 50 ng/mL | 6.0 | μL |
| IL-6 | 100 μg/mL | 50 ng/mL | 6.0 | μL |
| Flt3L | 100 μg/mL | 50 ng/mL | 6.0 | μL |
| EPO | 500 IU/mL | 10 IU/mL | 240 | μL |

b. The plate was taken from the incubator and placed on an inverted phase contrast microscope to observe cell growth and morphology.
c. Images were recorded at Day 4 as in Step 1.11.c. A 4× magnification of exemplary cells is shown in FIG. 8KK.
d. EBs and cells in medium from all wells of the TC-treated plate were collected into a 15 mL conical tube.
e. 1 mL Step 3 medium (1.13.a) was added to each well.
f. 15 mL conical tube containing EBs, cells, and medium was centrifuged at 500×g for 3 minutes at room temperature.
g. The supernatant was carefully aspirated and pelleted EBs and cells were gently resuspended in 6 mL fresh STEP 3 medium (1.13.a), and 1 mL was distributed to each well so that final volume per well of the 6-well TC-treated plate was 2 mL.
h. Cells continued to be incubated and cultured at 37° C., 5% CO₂. No agitation was required at this stage.
1.14 Day 6: Onset of hematopoietic differentiation—Endothelial to hematopoietic transition phase
   a. On Days 6 and 8, Advanced DMEM/F12 medium was pre-warmed at room temperature and, fresh STEP 4 medium was prepared:

| | Stock | Final | Volume for | |
|---|---|---|---|---|
| Reagent | Concentration | Concentration | 6-well plate | |
| Advanced DMEM/F12 | 100% | | 9.6 | mL |
| StemFit ® For | 100% | 20% | 2.4 | mL |
| Differentiation | | | | |
| L-Glutamine | 100X | 1X | 120 | μL |
| P/S | 100X | 0.5X-1X | 60 | μL |
| SCF | 100 μg/mL | 50 ng/mL | 6.0 | μL |
| IL-6 | 100 μg/mL | 50 ng/mL | 6.0 | μL |
| EPO | 500 IU/mL | 10 IU/mL | 240 | μL |

b. The plate was taken from the incubator and placed on an inverted phase contrast microscope to observe cell growth and morphology.
c. Images were recorded at Day 6 as in Step 1.11.c. A 4× magnification of exemplary cells is shown in FIG. 8LL.
d. EBs and cells in medium from all wells of the TC-treated plate were collected into a 15 mL conical tube.
e. 1 mL Step 4 medium (Step 1.14.a) was added to each well.
f. 15 mL conical tube containing EBs, cells, and medium was centrifuged at 500×g for 3 minutes at room temperature.
g. The supernatant was carefully aspirated and pelleted EBs and cells were gently resuspended in 6 mL fresh STEP 4 medium (Step 1.14.a), and 1 mL was distributed to each well so that final volume per well of the 6-well TC-treated plate was 2 mL.
h. Cells continued to be incubated and cultured at 37° C., 5% CO₂. No agitation was required at this stage.
i. At Day 8, STEP 4 medium (Step 1.14.a) was prepared.
j. The plate was taken from the incubator and placed on an inverted phase contrast microscope to observe cell growth and morphology.
k. Images were recorded at Day 8 as in 1.11.c. A 4× magnification of exemplary cells is shown in FIG. 8MM.
l. EBs and cells in medium from all wells of the TC plate were collected into a 15 mL conical tube.
m. 1 mL Step 4 medium (Step 1.14.a) was added to each well.
n. 15 mL conical tube containing EBs, cells, and medium was centrifuged at 500×g for 3 minutes at room temperature.
o. The supernatant was carefully aspirated and pelleted EBs and cells were gently resuspended in 6 mL fresh STEP 4 medium (Step 1.14.a), and 1 mL was distributed so that final volume per well of the 6-well TC-treated plate was 2 mL.
p. Cells continued to be incubated and cultured at 37° C., 5% CO₂. No agitation was required at this stage.
1.15 Day 10: Harvesting hematopoietic stem cells (iCD34/iHSCs) and preparing samples for flow cytometric analysis and cryopreservation was performed as follows:
   a. The plate was taken from the incubator and placed on an inverted phase contrast microscope to observe cell growth and morphology.
   b. Images were recorded at Day 10 as in Step 1.11.c. A 4× magnification of exemplary cells is shown in FIG. 8W.
   c. 50 mL of STEP 4 base medium (Step 1.14.a) without cytokines and growth factors were prepared.
   d. EBs and cells were gently pipetted up and down in the wells to ensure all EBs were in suspension. The suspended EBs and cells were transferred from all wells to a 15 mL conical tube.
   e. The EB and cell suspension was centrifuged at 300×g for 5 minutes at room temperature.
   f. The supernatant was carefully aspirated and 1 mL of Collagenase II (20 mg/mL or 2500 U/mL) was added to the pellet. The supernatant was gently pipetted up and down to mix. EBs and cells were incubated at 37° C., 5% CO₂ for 20 minutes.
   g. 3 mL of TryPLE Select were added to the EB and cell suspension and mixed by gently pipetting up and down. The EB and cell suspension was incubated at 37° C., 5% CO₂ for 20 minutes.
   h. The suspension was removed from the incubator and then gently mixed to break up any remaining clumps.
   i. 6 mL of STEP 4 base medium (Step 1.15.c) without cytokine and growth factors were added.
   j. The suspension was centrifuged at 300×g for 5 minutes at room temperature.
   k. The supernatant was carefully aspirated and the pellet was gently resuspended in 10 mL of STEP 4 base medium (Step 1.15.c) without cytokine and growth factors.
   l. The cell suspension was gently mixed and then passed through a 70 μm cell strainer followed by a 40 μm cell strainer.
   m. Cell Count was performed using NC-200 (Step 1.6).
   n. 0.5×10⁶ to 1.0×10⁶ cells were aliquoted for flow cytometric analysis using the iHSC panel.
   o. iHSC flow panel is shown in Table 12.
   p. Success criteria for iHSC redifferentiation step: purity of >70% Lineage marker-negative CD34+ cells and >0.5 fold expansion per input iPSC.
   q. iHSCs/iCD34 cells were either cryopreserved in CS-10 freezing medium as in Step 1.10.i.iv.a or were redifferentiated into iγδ T cells.
i. Note: Cryopreserved iHSC/iCD34 cells could be thawed and redifferentiated at a later date.

### LIST OF EMBODIMENTS

The following is a non-exhaustive list of embodiments contemplated by the present invention.

1. A method of de novo generation of hematopoietic stem cells (iHSCs) derived from human γδ T cell derived induced pluripotent stem cells (iPSCs) under serum-free and feeder-free culture conditions, said method comprising:
seeding human γδ T cell derived iPSCs in a tissue culture vessel wherein the iPSCs are maintained for up to about 6 days;
initiating redifferentiation of the iPSCs to iHSCs by culturing in the tissue culture vessel for about 2 days to obtain iPSC-derived cell intermediates;
culturing the iPSC-derived cell intermediates for about 8 days;
collecting non-adherent iPSC-derived cell intermediates and adding them back to the culture; and
harvesting redifferentiated iHSCs at about day 10 of culture into two different cellular fractions, a non-adherent cellular fraction and an adherent cellular fraction.

2. The method of item 1, further comprising:
the step of initiating redifferentiation of the iPSCs in a composition comprising basic fibroblast growth factor (bFGF), a glycogen synthase kinase inhibitor, a bone morphogenetic protein (BMP4), and vascular endothelial growth factor (VEGF).

3. The method of item 1, further comprising:
culturing the iPSC-derived cell intermediates for about 2 days in a composition comprising effective amounts of L-glutamine, an inhibitor of the Activin/BMP/TGFβ pathway), VEGF, bFGF, and, optionally, a stem cell factor (SCF) and/or antibiotic(s), and/or
culturing the iPSC-derived cell intermediates for about 2 days in a composition comprising L-glutamine, SCF, VEGF, FMS-like tyrosine kinase 3 ligand (Flt3L), erythropoietin (EPO), interleukin-3 (IL-3), interleukin-6 (IL-6), and optionally antibiotic(s), and/or
culturing the iPSC-derived cell intermediates for about 2 days in a composition comprising L-glutamine, SCF, IL-6, EPO, and optionally antibiotic(s), and/or
culturing redifferentiated iHSCs for about 2 days in a composition comprising L-glutamine and optionally antibiotic(s), wherein the composition does not comprise cytokines or growth factors.

4. A method of de novo generation of hematopoietic stem cells (iHSCs) derived from human γδ T cell derived induced pluripotent stem cells (iPSCs) under serum-free and feeder-free culture conditions, said method comprising:
i) obtaining one or more human γδ T cell derived iPSC, and
wherein the method further comprises one or more of the following steps:
a) seeding iPSCs at about 1.7×10³ cells/well to about 2.5×10³ cells/well of a six-well tissue culture vessel or an equivalent thereof;
b) maintaining the iPSCs under culture conditions for about 5.5 to about 6.5 days;
c) initiating redifferentiation of the iPSCs into iHSCs by culturing the iPSCs for about 1.8 days to about 2.2 days in a first cell culture medium to obtain iPSC-derived cell intermediates, wherein said first cell culture medium comprises StemFit® Basic 04 medium supplemented with basic fibroblast growth factor (bFGF) to a final concentration of about 90 ng/mL to about 110 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the iPSC-derived cell intermediates for an additional about 1.8 days to about 2.2 days in a second cell culture medium, wherein said second cell culture medium comprises Advanced DMEM/F12 medium and StemFit® For Differentiation medium present in about a 3.9 to 0.9 ratio to about a 4.1-1.1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
e) culturing the iPSC-derived cell intermediates for an additional about 1.8 days to about 2.2 days in a third cell culture medium, wherein said third cell culture medium comprises Advanced DMEM/F12 medium and StemFit® For Differentiation medium present in about a 3.9 to 0.9 ratio to about a 4.1-1.1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the iPSC-derived cell intermediates for an additional about 1.8 days to about 4.2 days in a fourth cell culture medium, wherein said fourth cell culture medium comprises Advanced DMEM/F12 medium and StemFit® For Differentiation medium present in about a 3.9 to 0.9 ratio to about a 4.1-1.1 ratio, and further comprises L-glutamine, SCF, IL-6, EPO, and optionally penicillin/streptomycin;
g) after about 7.8 days to about 8.2 days of redifferentiation, collecting non-adherent iPSC-derived cell intermediates from the fourth cell culture medium and adding them back to the culture in the fourth cell culture medium;
h) harvesting redifferentiated iHSCs at day 10 of culture in two different cellular fractions, a non-adherent cellular fraction and an adherent cellular fraction, wherein the redifferentiated iHSCs are harvested in a fifth cell culture medium, wherein the fifth cell culture medium comprises Advanced DMEM/F12 medium and StemFit® For Differentiation medium in about a 3.9 to 0.9 ratio to about a 4.1 to 1.1 ratio, and further comprises L-glutamine and optionally penicillin/streptomycin, wherein the fifth cell culture medium does not comprise cytokines or growth factors;
i) harvesting non-adherent iHSCs by centrifugation, wherein said harvesting optionally does not include further culturing;
j) harvesting adherent iHSCs by dispersing cells, optionally resuspending the cells obtained from dispersion, followed by passing through a first cell strainer and a second cell strainer, wherein the first cell strainer is 70 μm mesh size and the second cell strainer is 40 μm mesh size;
k) determining the purity of the iHSCs, said purity defined as CD34+ and lineage marker-negative;
l) not introducing serum in any of steps a-k;
n) not introducing any additional cells, including feeder cells, in any of steps a-m; or
o) obtaining de novo generated iHSCs.

5. A method of de novo generation of hematopoietic stem cells (iHSCs) derived from human γδ T cell derived induced pluripotent stem cell (iPSC) under serum-free and feeder-free culture conditions, said method comprising:
a) seeding the iPSCs at about 0.4×10⁶ cells/well to about 1.2×10⁶ cells/well of a six-well ultra low adhesion tissue culture vessel or an equivalent thereof;
b) culturing the iPSCs for up to about 48 hours with agitation to generate embryoid bodies (EBs);
c) transferring the EBs to a tissue culture treated culture vessel and initiating redifferentiation of the EBs for about 10 days, wherein the tissue vulture treated culture vessel comprises EBs and cells in suspension;
d) obtaining the EBs and cells in suspension;
e) optionally, treating the EBs and cells in suspension to obtain a single cell suspension.

6. The method of de novo generation of hematopoietic stem cells (iHSCs) derived from human γδ T cell derived induced pluripotent stem cell (iPSC) under serum-free and feeder-free culture conditions of item 5, said method further comprising:
providing CD34+ and lineage marker-negative iHSCs.

7. The method of de novo generation of hematopoietic stem cells (iHSCs) derived from human γδ T cell derived induced pluripotent stem cells (iPSCs) under serum-free and feeder-free culture conditions according to item 6, said method further comprising:
obtaining CD34+, and CD3−, and/or CD19−, and/or CD14−, and/or CD11b−, and/or CD11c−, and/or CD56−, and/or CD20−, and/or CD16−, and/or CD2−, and/or CD235a iHSCs.

8. A method of de novo generation of hematopoietic stem cells (iHSCs) derived from human γδ T cell derived induced pluripotent stem cells (iPSCs) under serum-free, feeder-free culture conditions, said method comprising:
i) obtaining one or more human γδ T cell derived iPSCs, and
wherein the method further comprises one or more of the following steps:
   a) seeding the iPSCs at about 0.55×10⁶ cells/well to about 0.65×10⁶ cells/well of a six-well ultra low adhesion tissue culture vessel or an equivalent thereof;
   b) maintaining iPSCs in culture conditions for about 44 hours to about 52 hours with agitation to generate embryoid bodies (EBs);
   c) transferring the EBs from the ultra low adhesion tissue culture vessel to a tissue culture treated tissue culture vessel and redifferentiating the EBs by culturing the EBs for about 1.8 days to about 2.2 days in a first cell culture medium, wherein said first cell culture medium comprises StemFit® Basic 04 medium supplemented with basic fibroblast growth factor (bFGF) to a final concentration of about 90 ng/mL to about 110 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
   d) culturing EBs and cells in suspension for an additional about 1.8 days to about 2.2 days in a second cell culture medium, wherein said second cell culture medium comprises Advanced DMEM/F12 medium and StemFit® For Differentiation medium present in about a 3.9 to 0.9 ratio to about a 4.1 to 1.1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
   e) culturing the EBs and cells in suspension for an additional about 1.8 days to about 2.2 days in a third cell culture medium, wherein said third cell culture medium comprises Advanced DMEM/F12 medium and StemFit® For Differentiation medium present in about a 3.9 to 0.9 ratio to about a 4.1 to 1.1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
   f) culturing the EBs and cells in suspension for an additional about 1.8 days to about 4.2 days in a fourth cell culture medium, wherein said fourth cell culture medium comprises Advanced DMEM/F12 medium and StemFit® For Differentiation medium present in about a 3.9 to 0.9 ratio to about a 4.1 to 1.1 ratio, and further comprises L-glutamine, SCF, IL-6, EPO, and optionally penicillin/streptomycin;
   g) after about 10 days of redifferentiation, harvesting the EBs and cells in suspension by centrifugation, thereby forming pelleted EBs and cells in suspension;
   h) resuspending the pelleted EBs and cells in suspension in a volume of 18 mg/mL to about 20 mg/mL collagenase type II and incubating the EBs and cells in suspension in the collagenase type II;
   i) adding TrypLE to the EBs and cells in suspension and incubating the EBs and cells in suspension to obtain a single cell suspension,
   j) mixing the single cell suspension;
   k) adding a fifth cell culture medium to the cell suspension, wherein said fifth cell culture medium comprises Advanced DMEM/F12 medium and StemFit® For Differentiation medium in a ratio of about a 3.9-0.9 ratio to about a 4.1-1.1 ratio, and further comprises L-glutamine and optionally penicillin/streptomycin, wherein the fifth cell culture medium does not comprise cytokines or growth factors,
   l) centrifuging the cell suspension to form a cell pellet and resuspending the cell pellet in the fifth cell culture medium to form a second cell suspension;
   m) passing the second cell suspension through a first cell strainer and a second cell strainer to obtain iHSCs, wherein the first cell strainer is 70 μm mesh size and the second cell strainer is 40 μm mesh size;
   n) determining the purity of the iHSCs, said purity defined as CD34+ and lineage marker-negative;
   o) not introducing serum in any of steps a-n;
   p) not introducing any additional cells, including feeder cells or stromal cells, in any of steps a-o; or
   q) obtaining de novo generated iHSCs.

9. The method of item 4 or item 8, wherein the cells are cultured under normoxic conditions.

10. The method of any one of items 4 and 8-9, further comprising further purifying and/or isolating the iHSCs.

11. The method of any one of items 4 and 8-10, wherein the obtained iHSCs are isolated differentiated cells or are capable of being further purified and/or isolated.

12. The method of any one of items 4 and 8-11, wherein the step of determining purity of the iHSCs comprises assaying for all of the lineage markers CD3, CD19, CD14, CD11b, CD11c, CD56, CD20, CD16, CD2, and CD235a via flow cytometry or an equivalent thereof.

13. The method of any one of items 4 and 8-12, wherein the lineage markers comprise one or more of CD3, CD19, CD14, CD11b, CD11c, CD56, CD20, CD16, CD2, and CD235a.

14. One or more cells obtained from a method of de novo generation of hematopoietic stem cells (iHSCs) derived from human γδ T cell derived induced pluripotent stem cells (iPSCs) under serum-free and feeder-free culture conditions, said method comprising the steps of any of items 1-13.

15. The one or more cells of item 14, wherein the step of determining purity of the iHSCs comprises assaying for all of the lineage markers CD3, CD19, CD14, CD11b, CD11c, CD56, CD20, CD16, CD2, and CD235a via flow cytometry or an equivalent thereof.

16. The one or more cells of any one of items 14 or 15, wherein the lineage markers comprise one or more of CD3, CD19, CD14, CD11b, CD11c, CD56, CD20, CD16, CD2, and CD235a.

17. The one or more cells of any one of items 14-16, wherein the one or more cells have high purity, viability, fold expansion, and potential to redifferentiate into immune effectors, including iPSC-derived γδ (iγδ) T cells.

18. A composition comprising the one or more cells of any one of items 14-17.

19. Use of the one or more cells of any one of items 14-17, in preparation of cells for treating a pathology, disease(s), in preparation of lymphocytes, in a bioreactor, in tissue engineering or in vitro drug screening for diseases.

20. A system for performing a method of de novo generation of hematopoietic stem cells (iHSCs) derived from human γδ T cell derived induced pluripotent stem cells (iPSCs) under serum-free and feeder-free culture conditions, said method comprising the steps of claims 1 or 5.

21. The system of item 20, wherein the process is performed by hand or with automated robotic assistance or a combination thereof.

22. The system of any one of items 20-21, wherein the process is automated or semi-automated.

23. The system of any one of items 20-22, comprising one or more software packages, the software package(s) operating and scheduling operation of the system.

24. The system of item 23, wherein the software package(s) are customized and or customizable for desired applications and are, optionally, menu-driven.

25. The system of item 22, wherein said automation comprises cell visualization, plate handling, plate coating, seeding, extraction, addition, cell feeding, incubation assays and or sampling.

26. The system of any one of items 20-25, comprising one or more incubators on line.

27. The system of any one of items 20-26, wherein the system comprises electronic humidity controls, a HEPA filter system, a carousel, said carousel comprising programmable stepping, oscillation cycles and or a two-way communication interface or any combination thereof.

28. The system of any one of items 20-27, wherein medium is added or removed or supplemented without disturbing or contaminating cells.

29. The system of any one of items 20-28, comprising one or more computer operated and controlled robotic arms.

30. The method of any one of items 4 and 8-13, wherein the method further comprises one or more of the following steps:
a) seeding the iPSCs at about 2×10³ cells/well of a six-well tissue culture vessel or an equivalent thereof;
b) maintaining the iPSCs under culture conditions for up to 6 days;
c) initiating redifferentiation of the iPSCs to iHSCs by culturing the iPSCs for about 2 days in the first cell culture medium to obtain iPSC-derived cell intermediates, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the iPSC-derived cell intermediates for an additional about 2 days in the second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
e) culturing the iPSC-derived cell intermediates for an additional about 2 days in the third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the iPSC-derived cell intermediates for an additional about 2 days to about 4 days in the fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin; or
g) after about 8 days of redifferentiation, collecting non-adherent iPSC-derived cell intermediates from the fourth cell culture medium and adding them back to the culture in the fourth cell culture medium;
h) harvesting redifferentiated iHSCs at day 10 of culture in two different cellular fractions, a non-adherent cellular fraction and an adherent cellular fraction, wherein the redifferentiated iHSCs are harvested in the fifth cell culture medium, wherein the fifth cell culture medium comprises Advanced DMEM/F12 medium and StemFit® For Differentiation medium in about a 4:1 ratio, and further comprises L-glutamine and optionally penicillin/streptomycin, wherein the fifth cell culture medium does not comprise cytokines or growth factors.

31. The method of any one of items 8-13, wherein the method further comprises:
a) seeding the iPSCs at about 0.6×10⁶ cells/well of a six-well ultra low adhesion tissue culture vessel or an equivalent thereof;
b) maintaining the iPSCs in culture conditions for up to 48 hours with agitation to generate embryoid bodies (EBs);
c) transferring the EBs from the ultra low adhesion tissue culture vessel to a tissue culture treated tissue culture vessel and redifferentiating the EBs by culturing the EBs for about 2 days in a first cell culture medium, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the EBs and cells in suspension for an additional about 2 days in the second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
e) culturing the EBs and cells in suspension for an additional about 2 days in the third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the EBs and cells in suspension for an additional about 2 days to about 4 days in the fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
g) after about 10 days of redifferentiation, harvesting the EBs and cells in suspension by centrifugation, thereby forming pelleted EBs and cells in suspension;
h) resuspending the pelleted EBs and cells in suspension in a volume of about 20 mg/mL collagenase type II and incubating the EBs and cells in suspension in the collagenase type II; or
k) adding the fifth cell culture medium to the cell suspension, wherein said fifth cell culture medium comprises Advanced DMEM/F12 medium and StemFit® For Differentiation medium in a ratio of about a 4:1 ratio, and further comprises L-glutamine and optionally penicillin/streptomycin, wherein the fifth cell culture medium does not comprise cytokines or growth factors.

32. The one or more cells of item 14, wherein the method of generating the one or more cells further comprises:
a) seeding the iPSCs at about 2×10³ cells/well of a six-well tissue culture vessel or an equivalent thereof;
b) maintaining the iPSCs under culture conditions for up to 6 days;
c) initiating redifferentiation of the iPSCs to iHSCs by culturing the iPSCs for about 2 days in the first cell culture medium to obtain iPSC-derived cell intermediates, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the iPSC-derived cell intermediates for an additional about 2 days in the second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
e) culturing the iPSC-derived cell intermediates for an additional about 2 days in the third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the iPSC-derived cell intermediates for an additional about 2 days to about 4 days in the fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
g) after about 8 days of redifferentiation, collecting non-adherent iPSC-derived cell intermediates from the fourth cell culture medium and adding them back to the culture in the fourth cell culture medium; or
h) harvesting redifferentiated iHSCs at day 10 of culture in two different cellular fractions, a non-adherent cellular fraction and an adherent cellular fraction, wherein the redifferentiated iHSCs are harvested in the fifth cell culture medium, wherein the fifth cell culture medium comprises Advanced DMEM/F12 medium and StemFit® For Differentiation medium in about a 4:1 ratio, and further comprises L-glutamine and optionally penicillin/streptomycin, wherein the fifth cell culture medium does not comprise cytokines or growth factors.

52. The one or more cells of item 14, wherein the method of generating the cells further comprises:
a) seeding the iPSCs at about 0.6×10⁶ cells/well of a six-well ultra low adhesion tissue culture vessel or an equivalent thereof;
b) maintaining the iPSCs in culture conditions for up to 48 hours with agitation to generate embryoid bodies (EBs);
c) transferring the EBs and cells in suspension from the ultra low adhesion tissue culture vessel to a tissue culture treated tissue culture vessel and redifferentiating the EBs by culturing the EBs for about 2 days in the first cell culture medium, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the EBs and cells in suspension for an additional about 2 days in the second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
e) culturing the EBs and cells in suspension for an additional about 2 days in the third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the EBs and cells in suspension for an additional about 2 days to about 4 days in the fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
g) after about 10 days of redifferentiation, harvesting the EBs and cells in suspension by centrifugation, thereby forming pelleted EBs and cells in suspension;
h) resuspending the pelleted EBs and cells in suspension in a volume of about 20 mg/mL collagenase type II and incubating the EBs and cells in suspension in the collagenase type II; or
k) adding the fifth cell culture medium to the cell suspension, wherein said fifth cell culture medium comprises Advanced DMEM/F12 medium and StemFit® For Differentiation medium in a ratio of about a 4:1 ratio, and further comprises L-glutamine and optionally penicillin/streptomycin, wherein the fifth cell culture medium does not comprise cytokines or growth factors.

53. The system of item 20,
wherein the method further comprises one or more of the following steps:
   a) seeding the iPSCs at about 2×10³ cells/well of a six-well tissue culture vessel or an equivalent thereof;
   b) maintaining the iPSCs under culture conditions for up to 6 days;
   c) initiating redifferentiation of the iPSCs to iHSCs by culturing the iPSCs for about 2 days in the first cell culture medium to obtain iPSC-derived cell intermediates, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
   d) culturing the iPSC-derived cell intermediates for an additional about 2 days in the second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
   e) culturing the iPSC-derived cell intermediates for an additional about 2 days in the third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
   f) culturing the iPSC-derived cell intermediates for an additional about 2 days to about 4 days in the fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
   g) after about 8 days of redifferentiation, collecting non-adherent iPSC-derived cell intermediates from the fourth cell culture medium and adding them back to the culture in the fourth cell culture medium; or
   h) harvesting redifferentiated iHSCs at day 10 of culture in two different cellular fractions, a non-adherent cellular fraction and an adherent cellular fraction, wherein the redifferentiated iHSCs are harvested in the fifth cell culture medium, wherein the fifth cell culture medium comprises Advanced DMEM/F12 medium and StemFit® For Differentiation medium in about a 4:1 ratio, and further comprises L-glutamine and optionally penicillin/streptomycin, wherein the fifth cell culture medium does not comprise cytokines or growth factors.

54. The system of item 20, wherein the method further comprises:
a) seeding the iPSCs at about 0.6×10⁶ cells/well of a six-well ultra low adhesion tissue culture vessel or an equivalent thereof;
b) maintaining the iPSCs in culture conditions for up to 48 hours with agitation to generate embryoid bodies (EBs);
c) transferring the EBs from the ultra low adhesion tissue culture vessel to a tissue culture treated tissue culture vessel and redifferentiating the EBs by culturing the EBs for about 2 days in the first cell culture medium, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing the EBs and cells in suspension for an additional about 2 days in the second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM, and optionally wherein SB431542 is diluted from a stock concentration of about 5 mM;
e) culturing the EBs and cells in suspension for an additional about 2 days in the third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the EBs and cells in suspension for an additional about 2 days to about 4 days in the fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
g) after about 10 days of redifferentiation, harvesting the EBs and cells in suspension by centrifugation, thereby forming pelleted EBs and cells in suspension;
h) resuspending the pelleted EBs and cells in suspension in a volume of about 20 mg/mL collagenase type II and incubating the EBs and cells in suspension in the collagenase type II; or
k) adding the fifth cell culture medium to the cell suspension, wherein said fifth cell culture medium comprises Advanced DMEM/F12 medium and StemFit® For Differentiation medium in a ratio of about a 4:1 ratio, and further comprises L-glutamine and optionally penicillin/streptomycin, wherein the fifth cell culture medium does not comprise cytokines or growth factors.

55. A method of de novo generation of hematopoietic stem cells (iHSCs) derived from human γδ T cell derived induced pluripotent stem cells (iPSCs) under serum-free, feeder-free culture conditions, said method comprising: a) a step for performing a function of initiating redifferentiation of the iPSCs to iHSCs by culturing in a tissue culture vessel for about 2 days to obtain iPSC-derived cell intermediates; (b) a step for performing a function of culturing the iPSC-derived cell intermediates for about 8 days; (c) a step for performing a function of collecting non-adherent iPSC-derived cell intermediates and adding them back to the culture; and (d) a step for performing a function of harvesting redifferentiated iHSCs at about day 10 of culture into two different cellular fractions, a non-adherent cellular fraction and an adherent cellular fraction.

56. A method of de novo generation of hematopoietic stem cells (iHSCs) derived from human γδ T cell derived induced pluripotent stem cells (iPSCs) under serum-free and feeder-free culture conditions, said method comprising: a) a step for performing a function of seeding the iPSCs; b) a step for performing a function of culturing the iPSCs for up to about 48 hours with agitation to generate embryoid bodies (EBs); c) a step for performing a function of transferring the EBs to a tissue culture treated culture vessel and initiating redifferentiation of the EBs for about 10 days, wherein the tissue culture treated culture vessel comprises EBs and cells in suspension; d) a step for performing a function of obtaining the EBs and cells in suspension; and e) optionally, a step for performing a function of treating the EBs and cells in suspension to obtain a single cell suspension.

57. A method of de novo generation of hematopoietic stem cells (iHSCs) derived from human γδ T cell derived induced pluripotent stem cells (iPSCs) under serum-free and feeder-free culture conditions, said method comprising: a) a step for performing a function of seeding the iPSCs; b) a step for performing a function of culturing the iPSCs for up to about 48 hours with agitation to generate embryoid bodies (EBs); c) a step for performing a function of transferring the EBs to a tissue culture treated culture vessel and initiating redifferentiation of the EBs for about 10 days, wherein the tissue culture treated culture vessel comprises EBs and cells in suspension; d) a step for performing a function of obtaining the EBs and cells in suspension; e) a step for performing a function of optionally, treating the EBs and cells in suspension to obtain a single cell suspension; and f) a step for performing a function of providing CD34+ and lineage marker-negative iHSCs.

58. A method of de novo generation of hematopoietic stem cells (iHSCs) derived from human γδ T cell derived induced pluripotent stem cells (iPSCs) under serum-free and feeder-free culture conditions, said method comprising: a) a step for performing a function of seeding the iPSCs; b) a step for performing a function of culturing the iPSCs for up to about 48 hours with agitation to generate embryoid bodies (EBs); c) a step for performing a function of transferring the EBs to a tissue culture treated culture vessel and initiating redifferentiation of the EBs for about 10 days, wherein the tissue culture treated culture vessel comprises EBs and cells in suspension; d) a step for performing a function of obtaining the EBs and cells in suspension; e) optionally, a step for performing a function of treating the EBs and cells in suspension to obtain a single cell suspension; and f) a step for performing a function of obtaining CD34+, and CD3−, and/or CD19−, and/or CD14−, and/or CD11b−, and/or CD11c−, and/or CD56−, and/or CD20−, and/or CD16−, and/or CD2−, and/or CD235a iHSCs.

59. An iHSC produced according to the method of any one of items 55-58.

60. One or more cells obtained from a method of de novo generation of hematopoietic stem cells (iHSCs) derived from human γδ T cell derived induced pluripotent stem cells (iPSCs) under serum-free and feeder-free culture conditions, said method comprising: a) a means for seeding human γδ T cell derived iPSCs in a tissue culture vessel wherein the iPSCs are maintained for up to about 6 days; b) a means for initiating redifferentiation of the iPSCs to iHSCs by culturing in the tissue culture vessel for about 2 days to obtain iPSC-derived cell intermediates; c) a means for culturing the iPSC-derived cell intermediates for about 8 days; d) a means for collecting non-adherent iPSC-derived cell intermediates and adding them back to the culture; and e) a means for harvesting redifferentiated iHSCs at about day 10 of culture into two different cellular fractions, a non-adherent cellular fraction and an adherent cellular fraction.

61. One or more cells obtained from a method of de novo generation of hematopoietic stem cells (iHSCs) derived from human γδ T cell derived induced pluripotent stem cells (iPSCs) under serum-free and feeder-free culture conditions, said method comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated vessel and initiating redifferentiation of the EBs for about 10 days, wherein the tissue culture treated culture vessel comprises EBs and cells in suspension; d) a means for obtaining the EBs and cells in suspension; and e) optionally, a means for treating the EBs and cells in suspension to obtain a single cell suspension.

62. One or more cells obtained from a method of de novo generation of hematopoietic stem cells (iHSCs) derived from human γδ T cell derived induced pluripotent stem cells (iPSCs) under serum-free and feeder-free culture conditions, said method comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated culture vessel and initiating redifferentiation of the EBs for about 10 days, wherein the tissue culture treated culture vessel comprises EBs and cells in suspension; d) a means for obtaining the EBs and cells in suspension; e) optionally, a means for treating the EBs and cells in suspension to obtain a single cell suspension; and f) a means for providing CD34+ and lineage marker-negative iHSCs.

63. One or more cells obtained from a method of de novo generation of hematopoietic stem cells (iHSCs) derived from human γδ T cell derived induced pluripotent stem cells (iPSCs) under serum-free and feeder-free culture conditions, said method comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated culture vessel and initiating redifferentiation of the EBs for about 10 days, wherein the tissue culture treated culture vessel comprises EBs and cells in suspension; d) a means for obtaining the EBs and cells in suspension; e) optionally, a means for treating the EBs and cells in suspension to obtain a single cell suspension; and f) a means for obtaining CD34+, and CD3−, and/or CD19−, and/or CD14−, and/or CD11b−, and/or CD11c−, and/or CD56−, and/or CD20−, and/or CD16−, and/or CD2−, and/or CD235a iHSCs.

64. A system for performing a method of de novo generation of hematopoietic stem cells (iHSCs) derived from human γδ T cell derived induced pluripotent stem cells (iPSCs) under serum-free and feeder-free culture conditions, said system comprising: a) a means for seeding human γδ T cell derived iPSCs in a tissue culture vessel wherein the iPSCs are maintained for up to about 6 days; b) a means for initiating redifferentiation of the iPSCs to iHSCs by culturing in the tissue culture vessel for about 2 days to obtain iPSC-derived cell intermediates; c) a means for culturing the iPSC-derived cell intermediates for about 8 days; d) a means for collecting non-adherent iPSC-derived cell intermediates and adding them back to the culture; and e) a means for harvesting redifferentiated iHSCs at about day 10 of culture into two different cellular fractions, a non-adherent cellular fraction and an adherent cellular fraction.

65. A system for performing a method of de novo generation of hematopoietic stem cells (iHSCs) derived from human γδ T cell derived induced pluripotent stem cells (iPSCs) under serum-free and feeder-free culture conditions, said system comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated culture vessel and initiating redifferentation of the EBs for about 10 days, wherein the tissue culture treated culture vessel comprises EBs and cells in suspension; d) a means for obtaining the EBs and cells in suspension; and e) optionally, a means for treating the EBs and cells in suspension to obtain a single cell suspension.

66. A system for performing a method of de novo generation of hematopoietic stem cells (iHSCs) derived from human γδ T cell derived induced pluripotent stem cells (iPSCs) under serum-free and feeder-free culture conditions, said system comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated culture vessel and initiating redifferentation of the EBs for about 10 days, wherein the tissue culture treated culture vessel comprises EBs and cells in suspension; d) a means for obtaining the EBs and cells in suspension; e) optionally, a means for treating the EBs and cells in suspension to obtain a single cell suspension; and f) a means for providing CD34+ and lineage marker-negative iHSCs.

67. A system for performing a method of de novo generation of hematopoietic stem cells (iHSCs) derived from human γδ T cell derived induced pluripotent stem cells (iPSCs) under serum-free and feeder-free culture conditions, said system comprising: a) a means for seeding the iPSCs at about 0.4×10⁶ cells/well to about 1.2×10⁶ cells/well of a six-well ultra low adhesion tissue culture vessel or an equivalent thereof; b) a means for culturing the iPSCs for up to about 48 hours with agitation to generate embryoid bodies (EBs); c) a means for transferring the EBs to a tissue culture treated vessel and initiating redifferentiation of the EBs for about 10 days, wherein the tissue culture treated culture vessel comprises EBs and cells in suspension; d) a means for obtaining the EBs and cells in suspension; e) optionally, a means for treating the EBs and cells in suspension to obtain a single cell suspension; and f) a means for obtaining CD34+, and CD3−, and/or CD19−, and/or CD14−, and/or CD11b−, and/or CD11c−, and/or CD56−, and/or CD20−, and/or CD16−, and/or CD2−, and/or CD235a iHSCs.

### REFERENCES

1. Montel-Hagen A, et al. From pluripotent stem cells to T cells. Exp Hematol. 2019; 71:24-31.
2. Gentles A J, et al. The prognostic landscape of genes and infiltrating immune cells across human cancers. Nat Med. 2015; 21:938-945
3. Watanabe D, et al. The generation of human γδ T cell-derived induced pluripotent stem cells from whole peripheral blood mononuclear cell culture. Stem Cells Transl Med. 2018; 7:34-44.
4. Williams R L, et al. Retention of epigenetic and transcriptional memory among reprogrammed T cells (T-iPSCs) imparts selective advantage for T cell re-differentiation. J Immunol. 2022; 208:107.
5. Cahan P, et al. Origins and implications of pluripotent stem cell variability and heterogeneity. Nat Rev Mol Cell Biol. 2013; 14:357-368.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of de novo generation of hematopoietic stem cells (iHSCs) derived from human γδ T cell derived induced pluripotent stem cells (iPSCs) under serum-free and feeder-free culture conditions, said method comprising:
seeding human γδ T cell derived iPSCs in a tissue culture vessel wherein the iPSCs are maintained for up to about 6 days;
initiating redifferentiation of the iPSCs to iHSCs by culturing in the tissue culture vessel for about 2 days to obtain iPSC-derived cell intermediates;
culturing the iPSC-derived cell intermediates for about 8 days;
collecting non-adherent iPSC-derived cell intermediates and adding them back to the culture; and
harvesting redifferentiated iHSCs at about day 10 of culture into two different cellular fractions, a non-adherent cellular fraction and an adherent cellular fraction.

2. The method of claim 1, further comprising:
the step of initiating redifferentiation of the iPSCs in a composition comprising basic fibroblast growth factor (bFGF), a glycogen synthase kinase inhibitor, a bone morphogenetic protein (BMP4), and vascular endothelial growth factor (VEGF).

3. The method of claim 1, further comprising:
culturing the iPSC-derived cell intermediates for about 2 days in a composition comprising effective amounts of L-glutamine, an inhibitor of the Activin/BMP/TGFβ pathway), VEGF, bFGF, and, optionally, a stem cell factor (SCF) and/or antibiotic(s), and/or
culturing the iPSC-derived cell intermediates for about 2 days in a composition comprising L-glutamine, SCF, VEGF, FMS-like tyrosine kinase 3 ligand (Flt3L), erythropoietin (EPO), interleukin-3 (IL-3), interleukin-6 (IL-6), and optionally antibiotic(s), and/or
culturing the iPSC-derived cell intermediates for about 2 days in a composition comprising L-glutamine, SCF, IL-6, EPO, and optionally antibiotic(s), and/or
culturing redifferentiated iHSCs for about 2 days in a composition comprising L-glutamine and optionally antibiotic(s), wherein the composition does not comprise cytokines or growth factors.

4. A method of de novo generation of hematopoietic stem cells (iHSCs) derived from human γδ T cell derived induced pluripotent stem cells (iPSCs) under serum-free and feeder-free culture conditions, said method comprising:
i) obtaining one or more human γδ T cell derived iPSC, and
wherein the method further comprises one or more of the following steps:
a) seeding iPSCs at about 2×10³ cells/well of a six-well tissue culture vessel or an equivalent thereof;
b) maintaining the iPSCs under culture conditions for up to 6 days;
c) initiating redifferentiation of the iPSCs to iHSCs by culturing the iPSCs for about 2 days in a first cell culture medium to obtain iPSC-derived cell intermediates, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing iPSC-derived cell intermediates for an additional about 2 days in a second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM;
e) culturing the iPSC-derived cell intermediates for an additional about 2 days in a third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the iPSC-derived cell intermediates for an additional about 2 days to about 4 days in a fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and optionally penicillin/streptomycin;
g) after about 8 days of redifferentiation, collecting non-adherent iPSC-derived cell intermediates from the fourth cell culture medium and adding them back to the culture in the fourth cell culture medium;
h) harvesting redifferentiated iHSCs at day 10 of culture in two different cellular fractions, a non-adherent cellular fraction and an adherent cellular fraction, wherein the redifferentiated iHSCs are harvested in a fifth cell culture medium, wherein the fifth cell culture medium comprises Advanced DMEM/F12 medium and StemFit® For Differentiation medium in about a 4:1 ratio, and further comprises L-glutamine and optionally penicillin/streptomycin, wherein the fifth cell culture medium does not comprise cytokines or growth factors;
i) harvesting non-adherent iHSCs by centrifugation, wherein said harvesting optionally does not include further culturing;
j) harvesting adherent iHSCs by dispersing cells, optionally resuspending the cells obtained from dispersion, followed by passing through a first cell strainer and a second cell strainer, wherein the first cell strainer is 70 μm mesh size and the second cell strainer is 40 μm mesh size;
k) determining the purity of the iHSCs, said purity defined as CD34+ and lineage marker-negative;
l) not introducing serum in any of steps a-k;
n) not introducing any additional cells, including feeder cells, in any of steps a-m; or
o) obtaining de novo generated iHSCs.

5. A method of de novo generation of hematopoietic stem cells (iHSCs) derived from human γδ T cell derived induced pluripotent stem cells (iPSCs) under serum-free and feeder-free culture conditions, said method comprising:
a) seeding the iPSCs at about 0.4×10⁶ cells/well to about 1.2×10⁶ cells/well of a six-well ultra low adhesion tissue culture vessel or an equivalent thereof;
b) culturing the iPSCs for up to about 48 hours with agitation to generate embryoid bodies (EBs);
c) transferring the EBs to a tissue culture treated culture vessel and initiating redifferentiation of the EBs for about 10 days, wherein the tissue culture treated culture vessel comprises EBs and cells in suspension;
d) obtaining the EBs and cells in suspension; and
e) optionally, treating the EBs and cells in suspension to obtain a single cell suspension.

6. The method of de novo generation of hematopoietic stem cells (iHSCs) derived from human γδ T cell derived induced pluripotent stem cells (iPSCs) under serum-free and feeder-free culture conditions according to claim 5, said method further comprising:
providing CD34+ and lineage marker-negative iHSCs.

7. The method of de novo generation of hematopoietic stem cells (iHSCs) derived from human γδ T cell derived induced pluripotent stem cells (iPSCs) under serum-free and feeder-free culture conditions according to claim 6, said method further comprising:
obtaining CD34+, and CD3−, and/or CD19−, and/or CD14−, and/or CD11b−, and/or CD11c−, and/or CD56−, and/or CD20−, and/or CD16−, and/or CD2−, and/or CD235a iHSCs.

8. A method of de novo generation of hematopoietic stem cells (iHSCs) derived from human γδ T cell derived induced pluripotent stem cells (iPSCs) under serum-free, feeder-free culture conditions, said method comprising:
i) obtaining one or more human γδ T cell derived iPSCs, and
wherein the method further comprises one or more of the following steps:
a) seeding the iPSCs at about 0.6×10⁶ cells/well of a six-well ultra low adhesion tissue culture vessel or an equivalent thereof;
b) maintaining the iPSCs in culture conditions for up to 48 hours with agitation to generate embryoid bodies (EBs);
c) transferring the EBs from the ultra low adhesion tissue culture vessel to a tissue culture treated tissue culture vessel and redifferentiating the EBs by culturing the EBs for about 2 days in a first cell culture medium, wherein said first cell culture medium comprises StemFit® Basic 04 medium supplemented with basic fibroblast growth factor (bFGF) to a final concentration of about 100 ng/mL bFGF, wherein said first cell culture medium further comprises CHIR99021 (glycogen synthase kinase inhibitor), bone morphogenetic protein 4 (BMP4), and recombinant human vascular endothelial growth factor (rhVEGF);
d) culturing EBs and cells in suspension for an additional about 2 days in a second cell culture medium, wherein said second cell culture medium comprises Advanced DMEM/F12 medium and StemFit® For Differentiation medium present in about a 4:1 ratio, and further comprises L-glutamine, SB431542 (inhibitor of Activin/BMP/TGFβ pathway), rhVEGF, bFGF, stem cell factor (SCF), and optionally penicillin/streptomycin, wherein SB431542 has a final concentration of about 1.5 μM to about 2.5 μM;
e) culturing the EBs and cells in suspension for an additional about 2 days in a third cell culture medium, wherein said third cell culture medium comprises Advanced DMEM/F12 medium and StemFit® For Differentiation medium present in about a 4:1 ratio, and further comprises L-glutamine, SCF, rhVEGF, FMS-like tyrosine kinase 3 ligand (Flt3L), erythropoietin (EPO), interleukin-3 (IL-3), interleukin-6 (IL-6), and optionally penicillin/streptomycin;
f) culturing the EBs and cells in suspension for an additional about 2 days to about 4 days in a fourth cell culture medium, wherein said fourth cell culture medium comprises Advanced DMEM/F12 medium and StemFit® For Differentiation medium present in about a 4:1 ratio, and further comprises L-glutamine, SCF, IL-6, EPO, and penicillin/streptomycin;
g) after about 10 days of redifferentiation, harvesting the EBs and cells in suspension by centrifugation, thereby forming pelleted EBs and cells in suspension;
h) resuspending the pelleted EBs and cells in suspension in a volume of about 20 mg/mL collagenase type II and incubating the EBs and cells in suspension in the collagenase type II;
i) adding TrypLE to the EBs and cells in suspension and incubating the EBs and cells in suspension to obtain a single cell suspension,
j) mixing the single cell suspension;
k) adding a fifth cell culture medium to the cell suspension, wherein said fifth cell culture medium comprises Advanced DMEM/F12 medium and StemFit® For Differentiation medium in a ratio of about a 4:1 ratio, and further comprises L-glutamine and optionally penicillin/streptomycin, wherein the fifth cell culture medium does not comprise cytokines or growth factors,
l) centrifuging the cell suspension to form a cell pellet and resuspending the cell pellet in the fifth cell culture medium to form a second cell suspension;
m) passing the second cell suspension through a first cell strainer and a second cell strainer to obtain iHSCs, wherein the first cell strainer is 70 μm mesh size and the second cell strainer is 40 μm mesh size;
n) determining the purity of the iHSCs, said purity defined as CD34+ and lineage marker-negative;
o) not introducing serum in any of steps a-n;
p) not introducing any additional cells, including feeder cells or stromal cells, in any of steps a-o; or
q) obtaining de novo generated iHSCs.

9. The method of claim 4 or claim 8, wherein the cells are cultured under normoxic conditions.

10. The method of any one of claims 4 and 8-9, further comprising further purifying and/or isolating the iHSCs.

11. The method of any one of claims 4 and 8-10, wherein the obtained iHSCs are isolated differentiated cells or are capable of being further purified and/or isolated.

12. The method of any one of claims 4 and 8-11, wherein the step of determining purity of the iHSCs comprises assaying for one or more, optionally all, of the lineage markers CD3, CD19, CD14, CD11b, CD11c, CD56, CD20, CD16, CD2, and CD235a via flow cytometry or an equivalent thereof.

13. The method of any one of claims 4 and 8-12, wherein the lineage markers comprise one or more of CD3, CD19, CD14, CD11b, CD11c, CD56, CD20, CD16, CD2, and CD235a.

14. One or more cells obtained from a method of de novo generation of hematopoietic stem cells (iHSCs) derived from human γδ T cell derived induced pluripotent stem cells (iPSCs) under serum-free and feeder-free culture conditions, said method comprising the steps of any one of claims 1-13.

15. The one or more cells of claim 14, wherein the one or more cells have high purity, viability, fold expansion, and potential to redifferentiate into immune effectors, including iPSC-derived γδ (iγδ) T cells.

16. The one or more cells of claim 14 or 15, wherein the step of determining purity of the iHSCs comprises assaying for one or more, optionally all, of the lineage markers CD3, CD19, CD14, CD11b, CD11c, CD56, CD20, CD16, CD2, and CD235a via flow cytometry or an equivalent thereof.

17. The one or more cells of any one of claims 14-16, wherein the lineage markers comprise one or more of CD3, CD19, CD14, CD11b, CD11c, CD56, CD20, CD16, CD2, and CD235a.

18. A composition comprising the one or more cells of any one of claims 14-17.

19. Use of the one or more cells of any one of claims 14-17, in preparation of cells for treating a pathology, disease(s), in preparation of lymphocytes, in a bioreactor, in tissue engineering or in vitro drug screening for diseases.

20. A system for performing a method of de novo generation of hematopoietic stem cells (iHSCs) derived from human γδ T cell derived induced pluripotent stem cells (iPSCs) under serum-free and feeder-free culture conditions, said method comprising the steps of claims 1 or 5.

21. The system of claim 20, wherein the method of claims 1 or 5 is performed by hand or with automated robotic assistance or a combination thereof.

22. The system of claims 20 or 21, wherein the system is automated or semi-automated.

23. The system of any one of claims 20-22, comprising one or more software packages, the software package(s) operating and scheduling operation of the system.

24. The system of claim 23, wherein the software package(s) are customized and or customizable for desired applications and are, optionally, menu-driven.

25. The system of claim 22, wherein said automation comprises cell visualization, plate handling, plate coating, seeding, extraction, addition, cell feeding, incubation assays and or sampling.

26. The system of any one of claims 20-25, comprising one or more incubators on line.

27. The system of any one of claims 20-26, wherein the system comprises electronic humidity controls, a HEPA filter system, a carousel, said carousel comprising programmable stepping, oscillation cycles and or a two-way communication interface or any combination thereof.

28. The system of any one of claims 20-27, wherein medium is added or removed or supplemented without disturbing or contaminating cells.

29. The system of any one of claims 20-28, comprising one or more computer operated and controlled robotic arms.

30. A method of de novo generation of hematopoietic stem cells (iHSCs) derived from human γδ T cell derived induced pluripotent stem cells (iPSCs) under serum-free, feeder-free culture conditions, said method comprising:
a step for performing a function of initiating redifferentiation of the iPSCs to iHSCs in a tissue culture vessel for about 2 days to obtain iPSC-derived cell intermediates;
a step for performing a function of culturing the iPSC-derived cell intermediates for about 8 days;
a step for performing a function of collecting non-adherent iPSC-derived cell intermediates and adding them back to the culture; and
a step for performing a function of harvesting redifferentiated iHSCs at about day 10 of culture into two different cellular fractions, a non-adherent cellular fraction and an adherent cellular fraction.

31. A method of de novo generation of hematopoietic stem cells (iHSCs) derived from human γδ T cell derived induced pluripotent stem cells (iPSCs) under serum-free and feeder-free culture conditions, said method comprising:
a) a step for performing a function of seeding the iPSCs;
b) a step for performing a function of culturing the iPSCs for up to about 48 hours with agitation to generate embryoid bodies (EBs);
c) a step for performing a function of transferring the EBs to a tissue culture treated culture vessel and initiating redifferntiation of the EBs for about 10 days, wherein the tissue culture treated culture vessel comprises EBs and cells in suspension;
d) a step for performing a function of obtaining the EBs and cells in suspension; and
e) optionally, a step for performing a function of treating the EBs and cells in suspension to obtain a single cell suspension.

32. A method of de novo generation of hematopoietic stem cells (iHSCs) derived from human γδ T cell derived induced pluripotent stem cells (iPSCs) under serum-free and feeder-free culture conditions, said method comprising:
a) a step for performing a function of seeding the iPSCs;
b) a step for performing a function of culturing the iPSCs for up to about 48 hours with agitation to generate embryoid bodies (EBs);
c) a step for performing a function of transferring the EBs to a tissue culture treated culture vessel and initiating redifferentiation of the EBs for about 10 days, wherein the tissue culture treated culture vessel comprises EBs and cells in suspension;
d) a step for performing a function of obtaining the EBs and cells in suspension;
e) a step for performing a function of optionally, treating the EBs and cells in suspension to obtain a single cell suspension; and
f) a step for performing a function of providing CD34+ and lineage marker-negative iHSCs.

33. A method of de novo generation of hematopoietic stem cells (iHSCs) derived from human γδ T cell derived induced pluripotent stem cells (iPSCs) under serum-free and feeder-free culture conditions, said method comprising:
a) a step for performing a function of seeding the iPSCs;
b) a step for performing a function of culturing the iPSCs for up to about 48 hours with agitation to generate embryoid bodies (EBs);
c) a step for performing a function of transferring the EBs to a tissue culture treated vessel and initiating redifferentiation of the EBs for about 10 days, wherein the tissue culture treated culture vessel comprises EBs and cells in suspension;
d) a step for performing a function of obtaining the EBs and cells in suspension;
e) optionally, a step for performing a function of treating the EBs and cells in suspension to obtain a single cell suspension; and
f) a step for performing a function of obtaining CD34+, and CD3⁻, and/or CD19⁻, and/or CD14⁻, and/or CD11b⁻, and/or CD11c−, and/or CD56⁻, and/or CD20⁻, and/or CD16⁻, and/or CD2⁻, and/or CD235a⁻ iHSCs.

34. An iHSC produced according to the method of any one of claims 30-33.

35. One or more cells obtained from a method of de novo generation of hematopoietic stem cells (iHSCs) derived from human γδ T cell derived induced pluripotent stem cells (iPSCs) under serum-free and feeder-free culture conditions, said method comprising:
a means for seeding human γδ T cell derived iPSCs in a tissue culture vessel wherein the iPSCs are maintained for up to about 6 days;
a means for initiating redifferentiation of the iPSCs to iHSCS by culturing in the tissue culture vessel for about 2 days to obtain iPSC-derived cell intermediates;
a means for culturing the iPSC-derived cell intermediates for about 8 days;
a means for collecting non-adherent iPSC-derived cell intermediates and adding them back to the culture; and
a means for harvesting redifferentiated iHSCs at about day 10 of culture into two different cellular fractions, a non-adherent cellular fraction and an adherent cellular fraction.
